SEQUENCE LISTING (30 sequences)

SEQ ID NO: 1 - protein, 1675 aa, Homo sapiens
  ref <308>: DATABASE ACCESSION NUMBER: UniProtKB/Q00610
  ref <309>: DATABASE ENTRY DATE: 2009-05-26
  ref <313>: RELEVANT RESIDUES IN SEQ ID NO: (1)..(1675)
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGQPQLMLTAGPSVAVPPQAPFGYGYTAPPYGQPQPGFGYSM

SEQ ID NO: 2 - protein, 1640 aa, Homo sapiens
  ref <308>: DATABASE ACCESSION NUMBER: UniProtKB/P53675
  ref <309>: DATABASE ENTRY DATE: 2009-05-26
  ref <313>: RELEVANT RESIDUES IN SEQ ID NO: (1)..(1640)
MAQILPVRFQEHFQLQNLGINPANIGFSTLTMESDKFICIREKVGEQAQVTIIDMSDPMAPIRRPISAESAIMNPASKVIALKAGKTLQIFNIEMKSKMKAHTMAEEVIFWKWVSVNTVALVTETAVYHWSMEGDSQPMKMFDRHTSLVGCQVIHYRTDEYQKWLLLVGISAQQNRVVGAMQLYSVDRKVSQPIEGHAAAFAEFKMEGNAKPATLFCFAVRNPTGGKLHIIEVGQPAAGNQPFVKKAVDVFFPPEAQNDFPVAMQIGAKHGVIYLITKYGYLHLYDLESGVCICMNRISADTIFVTAPHKPTSGIIGVNTKGQVLSVCVEEDNIVNYATNVLQNPDLGLRLAVRSNLAGAEKLFVRKFNTLFAQGSYAEAAKVAASAPKGILRTRETVQKFQSIPAQSGQASPLLQYFGILLDQGQLNKLESLELCHLVLQQGRKQLLEKWLKEDKLECSEELGDLVKTTDPMLALSVYLRANVPSKVIQCFAETGQFQKIVLYAKKVGYTPDWIFLLRGVMKISPEQGLQFSRMLVQDEEPLANISQIVDIFMENSLIQQCTSFLLDALKNNRPAEGLLQTWLLEMNLVHAPQVADAILGNKMFTHYDRAHIAQLCEKAGLLQQALEHYTDLYDIKRAVVHTHLLNPEWLVNFFGSLSVEDSVECLHAMLSANIRQNLQLCVQVASKYHEQLGTQALVELFESFKSYKGLFYFLGSIVNFSQDPDVHLKYIQAACKTGQIKEVERICRESSCYNPERVKNFLKEAKLTDQLPLIIVCDRFGFVHDLVLYLYRNNLQRYIEIYVQKVNPSRTPAVIGGLLDVDCSEEVIKHLIMAVRGQFSTDELVAEVEKRNRLKLLLPWLESQIQEGCEEPATHNALAKIYIDSNNSPECFLRENAYYDSSVVGRYCEKRDPHLACVAYERGQCDLELIKVCNENSLFKSEARYLVCRKDPELWAHVLEETNPSRRQLIDQVVQTALSETRDPEEISVTVKAFMTADLPNELIELLEKIVLDNSVFSEHRNLQNLLILTAIKADRTRVMEYISRLDNYDALDIASIAVSSALYEEAFTVFHKFDMNASAIQVLIEHIGNLDRAYEFAERCNEPAVWSQLAQAQLQKDLVKEAINSYIRGDDPSSYLEVVQSASRSNNWEDLVKFLQMARKKGRESYIETELIFALAKTSRVSELEDFINGPNNAHIQQVGDRCYEEGMYEAAKLLYSNVSNFARLASTLVHLGEYQAAVDNSRKASSTRTWKEVCFACMDGQEFRFAQLCGLHIVIHADELEELMCYYQDRGYFEELILLLEAALGLERAHMGMFTELAILYSKFKPQKMLEHLELFWSRVNIPKVLRAAEQAHLWAELVFLYDKYEEYDNAVLTMMSHPTEAWKEGQFKDIITKVANVELCYRALQFYLDYKPLLINDLLLVLSPRLDHTWTVSFFSKAGQLPLVKPYLRSVQSHNNKSVNEALNHLLTEEEDYQGLRASIDAYDNFDNISLAQQLEKHQLMEFRCIAAYLYKGNNWWAQSVELCKKDHLYKDAMQHAAESRDAELAQKLLQWFLEEGKRECFAACLFTCYDLLRPDMVLELAWRHNLVDLAMPYFIQVMREYLSKVDKLDALESLRKQEEHVTEPAPLVFDFDGHE

SEQ ID NO: 3 - protein, 1583 aa, Homo sapiens
  ref <308>: DATABASE ACCESSION NUMBER: NCBI/EAX03047
  ref <309>: DATABASE ENTRY DATE: 2006-12-18
  ref <313>: RELEVANT RESIDUES IN SEQ ID NO: (1)..(1583)
MAQILPVRFQEHFQLQNLGINPANIGFSTLTMESDKFICIREKVGEQAQVTIIDMSDPMAPIRRPISAESAIMNPASKVIALKAGKTLQIFNIEMKSKMKAHTMAEEVIFWKWVSVNTVALVTETAVYHWSMEGDSQPMKMFDRHTSLVGCQVIHYRTDEYQKWLLLVGISAQQNRVVGAMQLYSVDRKVSQPIEGHAAAFAEFKMEGNAKPATLFCFAVRNPTGGKLHIIEVGQPAAGNQPFVKKAVDVFFPPEAQNDFPVAMQIGAKHGVIYLITKYGYLHLYDLESGVCICMNRISADTIFVTAPHKPTSGIIGVNKKGQVLSVCVEEDNIVNYATNVLQNPDLGLRLAVRSNLAGAEKLFVRKFNTLFAQGSYAEAAKVAASAPKGILRTRETVQKFQSIPAQSGQASPLLQYFGILLDQGQLNKLESLELCHLVLQQGRKQLLEKWLKEDKLECSEELGDLVKTTDPMLALSVYLRANVPSKVIQCFAETGQFQKIVLYAKKVGYTPDWIFLLRGVMKISPEQGLQFSRMLVQDEEPLANISQIVDIFMENSLIQQCTSFLLDALKNNRPAEGLLQTWLLEMNLVHAPQVADAILGNKMFTHYDRAHIAQLCEKAGLLQQALEHYTDLYDIKRAVVHTHLLNPEWLVNFFGSLSVEDSVECLHAMLSANIRQNLQLCVQVASKYHEQLGTQALVELFESFKSYKGLFYFLGSIVNFSQDPDVHLKYIQAACKTGQIKEVERICRESSCYNPERVKNFLKEAKLTDQLPLIIVCDRFGFVHDLVLYLYRNNLQRYIEIYVQKVNPSRTPAVIGGLLDVDCSEEVIKHLIMAVRGQFSTDELVAEVEKRNRLKLLLPWLESQIQEGCEEPATHNALAKIYIDSNNSPECFLRENAYYDSSVVGRYCEKRDPHLACVAYERGQCDLELIKVCNENSLFKSEARYLVCRKDPELWAHVLEETNPSRRQLIDQVVQTALSETRDPEEISVTVKAFMTADLPNELIELLEKIVLDNSVFSEHRNLQNLLILTAIKADRTRVMEYISRLDNYDALDIASIAVSSALYEEAFTVFHKFDMNASAIQVLIEHIGNLDRAYEFAERCNEPAVWSQLAQAQLQKDLVKEAINSYIRGDDPSSYLEVVQSASRSNNWEDLVKFLQMARKKGRESYIETELIFALAKTSRVSELEDFINGPNNAHIQQVGDRCYEEGMYEAAKLLYSNVSNFARLASTLVHLGEYQAAVDNSRKASSTRTWKEVCFACMDGQEFRFAQLCGLHIVIHADELEELMCYYQDRGYFEELILLLEAALGLERAHMGMFTELAILYSKFKPQKMLEHLELFWSRVNIPKVLRAAEQAHLWAELVFLYDKYEEYDNAVLTMMSHPTEAWKEGQFKDIITKVANVELCYRALQFYLDYKPLLINDLLLVLSPRLDHTWTVSFFSKAGQLPLVKPYLRSVQSHNNKSVNEALNHLLTEEEDYQDAMQHAAESRDAELAQKLLQWFLEEGKRECFAACLFTCYDLLRPDMVLELAWRHNLVDLAMPYFIQVMREYLSKVDKLDALESLRKQEEHVTEPAPLVFDFDGHE

SEQ ID NO: 4 - protein, 1685 aa, Homo sapiens
  ref <308>: DATABASE ACCESSION NUMBER: NCBI/BAA04801
  ref <309>: DATABASE ENTRY DATE: 2004-01-10
  ref <313>: RELEVANT RESIDUES IN SEQ ID NO: (1)..(1685)
QEETITPDSAMAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGQPQLMLTAGPSVAVPPQAPFGYGYTAPPYGQPQPGFGYSM

SEQ ID NO: 5 - protein, 1682 aa, Homo sapiens
  ref <308>: DATABASE ACCESSION NUMBER: NCBI/EAW94395
  ref <309>: DATABASE ENTRY DATE: 2006-12-18
  ref <313>: RELEVANT RESIDUES IN SEQ ID NO: (1)..(1682)
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDAIKEKVDKLDASESLRKEEEQATETQPIVYGQPQLMLTAGPSVAVPPQAPFGYGYTAPPYGQPQPGFGYSM

SEQ ID NO: 6 - protein, 1675 aa, Homo sapiens; identical to SEQ ID NO: 1
  ref <308>: DATABASE ACCESSION NUMBER: NCBI/EAW94399
  ref <309>: DATABASE ENTRY DATE: 2006-12-18
  ref <313>: RELEVANT RESIDUES IN SEQ ID NO: (1)..(1675)

SEQ ID NO: 7 - protein, 1679 aa, Homo sapiens
  ref <308>: DATABASE ACCESSION NUMBER: NCBI/EAW94397
  ref <309>: DATABASE ENTRY DATE: 2006-12-18
  ref <313>: RELEVANT RESIDUES IN SEQ ID NO: (1)..(1679)
MAQILPIRFQEHLQLQNLGINPANIGFSTLTMESDKFICIREKVGEQAQVVIIDMNDPSNPIRRPISADSAIMNPASKVIALKGIKESGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGQPQLMLTAGPSVAVPPQAPFGYGYTAPPYGQPQPGFGYSM

SEQ ID NO: 8 - protein, 1569 aa, Homo sapiens
  ref <308>: DATABASE ACCESSION NUMBER: NCBI/AAB40909
  ref <309>: DATABASE ENTRY DATE: 1997-01-15
  ref <313>: RELEVANT RESIDUES IN SEQ ID NO: (1)..(1569)
MAQILPVRFQEHFQLQNLGINPANIGFSTLTMESDKFICIREKVGEQAQVTIIDMSDPMAPIRRPISAESAIMNPASKVIALKAGKTLQIFNIEMKSKMKAHTMAEEVIFWKWVSVNTVALVTETAVYHWSMEGDSQPMKMFDRHTSLVGCQVIHYRTDEYQKWLLLVGISAQQNRVVGAMQLYSVDRKVSQPIEGHAAAFAEFKMEGNAKPATLFCFAVRNPTGGKLHIIEVGQPAAGNQPFVKKAVDVFFPPEAQNDFPVAMQIGAKHGVIYLITKYGYLHLYDLESGVCICMNRISADTIFVTAPHKPTSGIIGVNKKGQVLSVCVEEDNIVNYATNVLQNPDLGLRLAVRSNLAGAEKLFVRKFNTLFAQGSYAEAAKVAASAPKGILRTRETVQKFQSIPAQSGQASPLLQYFGILLDQGQLNKLESLELCHLVLQQGRKQLLEKWLKEDKLECSEELGDLVKTTDPMLALSVYLRANVPSKVIQCFAETGQFQKIVLYAKKVGYTPDWIFLLRGVMKISPEQGLQFSRMLVQDEEPLANISQIVDIFMENSLIQQCTSFLLDALKNNRPAEGLLQTWLLEMNLVHAPQVADAILGNKMFTHYDRAHIAQLCEKAGLLQQALEHYTDLYDIKRAVVHTHLLNPEWLVNFFGSLSVEDSVECLHAMLSANIRQNLQLCVQVASKYHKQLGTQALVELFESFKSYKGLFYFLGSIVNFSQDPDVHLKYIQAACKTGQIKEVERICRESSCYNPERVKNFLKEAKLTDQLPLIIVCDRFGFVHDLVLYLYRNNLQRYIEIYVQKVNPSRTPAVIGGLLDVDCSEEVIKHLIMAVRGQFSTDELVAEVEKRNRLKLLLPWLESQIQEGCEEPATHNALAKIYIDSNNSPECFLRENAYYDSSVVGRYCEKRDPHLACVAYERGQCDLELIKVCNENSLFKSEARYLVCRKDPELWAHVLEETNPSRRQLIDQVVQTALSETRDPEEISVTVKAFMTADLPNELIELLEKIVLDNSVFSEHRNLQNLLILTAIKADRTRVMEYISRLDNYDALDIASIAVSSALYEEAFTVFHKFDMNASAIQVLIEHIGNLDRAYEFAERCNEPAVWSQLAQAQLQKDLVKEAINSYIRGDDPSSYLEVVQSASRSNNWEDLVKFLQMARKKGRESYIETELIFALAKTSRVSELEDFINGPNNAHIQQVGDRCYEEGMYEAAKLLYSNVSNFARLASTLVHLGEYQAAVDNSRKASSTRTWKEVCFACMDGQEFRFAQLCGLHIVIHADELEELMCYYQDRGYFEELILLLEAALGLERAHMGMFTELAILYSKFKPQKMLEHLELFWSRVNIPKVLRAAEQAHLWAELVFLYDKYEEYDNAVLTMMSHPTEAWKEGQFKDIITKVANVELCYRALQFYLDYKPLLINDLLLVLSPRLDHTWTVSFFSKAGQLPLVKPYLRSVQSHNNKSVNEALNHLLTEKEDYQDAMQHAAESRDAELAQKLLQWFLEEGKRECFAACLFTCYDLLRPDMVLELAWRHNLVDLAMPYFIQVMREYLSKVDKLDALESLPPSKRSM

SEQ ID NO: 9 - protein, 1639 aa, Homo sapiens
  ref <308>: DATABASE ACCESSION NUMBER: NCBI/AAH51800
  ref <309>: DATABASE ENTRY DATE: 2006-10-06
  ref <313>: RELEVANT RESIDUES IN SEQ ID NO: (1)..(1639)
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGNLSL

SEQ ID NO: 10 - protein, 1626 aa, Homo sapiens
  ref <308>: DATABASE ACCESSION NUMBER: NCBI/AAB40908
  ref <309>: DATABASE ENTRY DATE: 1997-01-15
  ref <313>: RELEVANT RESIDUES IN SEQ ID NO: (1)..(1626)
MAQILPVRFQEHFQLQNLGINPANIGFSTLTMESDKFICIREKVGEQAQVTIIDMSDPMAPIRRPISAESAIMNPASKVIALKAGKTLQIFNIEMKSKMKAHTMAEEVIFWKWVSVNTVALVTETAVYHWSMEGDSQPMKMFDRHTSLVGCQVIHYRTDEYQKWLLLVGISAQQNRVVGAMQLYSVDRKVSQPIEGHAAAFAEFKMEGNAKPATLFCFAVRNPTGGKLHIIEVGQPAAGNQPFVKKAVDVFFPPEAQNDFPVAMQIGAKHGVIYLITKYGYLHLYDLESGVCICMNRISADTIFVTAPHKPTSGIIGVNKKGQVLSVCVEEDNIVNYATNVLQNPDLGLRLAVRSNLAGAEKLFVRKFNTLFAQGSYAEAAKVAASAPKGILRTRETVQKFQSIPAQSGQASPLLQYFGILLDQGQLNKLESLELCHLVLQQGRKQLLEKWLKEDKLECSEELGDLVKTTDPMLALSVYLRANVPSKVIQCFAETGQFQKIVLYAKKVGYTPDWIFLLRGVMKISPEQGLQFSRMLVQDEEPLANISQIVDIFMENSLIQQCTSFLLDALKNNRPAEGLLQTWLLEMNLVHAPQVADAILGNKMFTHYDRAHIAQLCEKAGLLQQALEHYTDLYDIKRAVVHTHLLNPEWLVNFFGSLSVEDSVECLHAMLSANIRQNLQLCVQVASKYHKQLGTQALVELFESFKSYKGLFYFLGSIVNFSQDPDVHLKYIQAACKTGQIKEVERICRESSCYNPERVKNFLKEAKLTDQLPLIIVCDRFGFVHDLVLYLYRNNLQRYIEIYVQKVNPSRTPAVIGGLLDVDCSEEVIKHLIMAVRGQFSTDELVAEVEKRNRLKLLLPWLESQIQEGCEEPATHNALAKIYIDSNNSPECFLRENAYYDSSVVGRYCEKRDPHLACVAYERGQCDLELIKVCNENSLFKSEARYLVCRKDPELWAHVLEETNPSRRQLIDQVVQTALSETRDPEEISVTVKAFMTADLPNELIELLEKIVLDNSVFSEHRNLQNLLILTAIKADRTRVMEYISRLDNYDALDIASIAVSSALYEEAFTVFHKFDMNASAIQVLIEHIGNLDRAYEFAERCNEPAVWSQLAQAQLQKDLVKEAINSYIRGDDPSSYLEVVQSASRSNNWEDLVKFLQMARKKGRESYIETELIFALAKTSRVSELEDFINGPNNAHIQQVGDRCYEEGMYEAAKLLYSNVSNFARLASTLVHLGEYQAAVDNSRKASSTRTWKEVCFACMDGQEFRFAQLCGLHIVIHADELEELMCYYQDRGYFEELILLLEAALGLERAHMGMFTELAILYSKFKPQKMLEHLELFWSRVNIPKVLRAAEQAHLWAELVFLYDKYEEYDNAVLTMMSHPTEAWKEGQFKDIITKVANVELCYRALQFYLDYKPLLINDLLLVLSPRLDHTWTVSFFSKAGQLPLVKPYLRSVQSHNNKSVNEALNHLLTEKEDYQGLRASIDAYDNFDNISLAQQLEKHQLMEFRCIAAYLYKGNNWWAQSVELCKKDHLYKDAMQHAAESRDAELAQKLLQWFLEEGKRECFAACLFTCYDLLRPDMVLELAWRHNLVDLAMPYFIQVMREYLSKVDKLDALESLPPSKRSM

SEQ ID NO: 11 - protein, 1639 aa, Homo sapiens; identical to SEQ ID NO: 9
  ref <308>: DATABASE ACCESSION NUMBER: NCBI/EAW94398
  ref <309>: DATABASE ENTRY DATE: 2006-12-18
  ref <313>: RELEVANT RESIDUES IN SEQ ID NO: (1)..(1639)

SEQ ID NO: 12 - protein, 248 aa, Homo sapiens
  ref <308>: DATABASE ACCESSION NUMBER: UniProtKB/P09496
  ref <309>: DATABASE ENTRY DATE: 2009-05-26
  ref <313>: RELEVANT RESIDUES IN SEQ ID NO: (1)..(248)
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRVADEAFYKQPFADVIGYVTNINHPCYSLEQAAEEAFVNDIDESSPGTEWERVARLCDFNPKSSKQAKDVSRMRSVLISLKQAPLVH

SEQ ID NO: 13 - protein, 229 aa, Homo sapiens
  ref <308>: DATABASE ACCESSION NUMBER: UniProtKB/P09497
  ref <309>: DATABASE ENTRY DATE: 2009-05-26
  ref <313>: RELEVANT RESIDUES IN SEQ ID NO: (1)..(229)
MADDFGFFSSSESGAPEAAEEDPAAAFLAQQESEIAGIENDEGFGAPAGSHAAPAQPGPTSGAGSEDMGTTVNGDVFQEANGPADGYAAIAQADRLTQEPESIRKWREEQRKRLQELDAASKVTEQEWREKAKKDLEEWNQRQSEQVEKNKINNRIADKAFYQQPDADIIGYVASEEAFVKESKEETPGTEWEKVAQLCDFNPKSSKQCKDVSRLRSVLMSLKQTPLSR

SEQ ID NO: 14 - protein, 236 aa, Homo sapiens
  ref <308>: DATABASE ACCESSION NUMBER: NCBI/NP_001070145
  ref <309>: DATABASE ENTRY DATE: 2008-05-01
  ref <313>: RELEVANT RESIDUES IN SEQ ID NO: (1)..(236)
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRVADEAFYKQPFADVIGYVAAEEAFVNDIDESSPGTEWERVARLCDFNPKSSKQAKDVSRMRSVLISLKQAPLVH

SEQ ID NO: 15 - protein, 1224 aa, Homo sapiens
  ref <308>: DATABASE ACCESSION NUMBER: UniProtKB/P53621
  ref <309>: DATABASE ENTRY DATE: 2009-05-26
  ref <313>: RELEVANT RESIDUES IN SEQ ID NO: (1)..(1224)
MLTKFETKSARVKGLSFHPKRPWILTSLHNGVIQLWDYRMCTLIDKFDEHDGPVRGIDFHKQQPLFVSGGDDYKIKVWNYKLRRCLFTLLGHLDYIRTTFFHHEYPWILSASDDQTIRVWNWQSRTCVCVLTGHNHYVMCAQFHPTEDLVVSASLDQTVRVWDISGLRKKNLSPGAVESDVRGITGVDLFGTTDAVVKHVLEGHDRGVNWAAFHPTMPLIVSGADDRQVKIWRMNESKAWEVDTCRGHYNNVSCAVFHPRQELILSNSEDKSIRVWDMSKRTGVQTFRRDHDRFWVLAAHPNLNLFAAGHDGGMIVFKLERERPAYAVHGNMLHYVKDRFLRQLDFNSSKDVAVMQLRSGSKFPVFNMSYNPAENAVLLCTRASNLENSTYDLYTIPKDADSQNPDAPEGKRSSGLTAVWVARNRFAVLDRMHSLLIKNLKNEITKKVQVPNCDEIFYAGTGNLLLRDADSITLFDVQQKRTLASVKISKVKYVIWSADMSHVALLAKHAIVICNRKLDALCNIHENIRVKSGAWDESGVFIYTTSNHIKYAVTTGDHGIIRTLDLPIYVTRVKGNNVYCLDRECRPRVLTIDPTEFKFKLALINRKYDEVLHMVRNAKLVGQSIIAYLQKKGYPEVALHFVKDEKTRFSLALECGNIEIALEAAKALDDKNCWEKLGEVALLQGNHQIVEMCYQRTKNFDKLSFLYLITGNLEKLRKMMKIAEIRKDMSGHYQNALYLGDVSERVRILKNCGQKSLAYLTAATHGLDEEAESLKETFDPEKETIPDIDPNAKLLQPPAPIMPLDTNWPLLTVSKGFFEGTIASKGKGGALAADIDIDTVGTEGWGEDAELQLDEDGFVEATEGLGDDALGKGQEEGGGWDVEEDLELPPELDISPGAAGGAEDGFFVPPTKGTSPTQIWCNNSQLPVDHILAGSFETAMRLLHDQVGVIQFGPYKQLFLQTYARGRTTYQALPCLPSMYGYPNRNWKDAGLKNGVPAVGLKLNDLIQRLQLCYQLTTVGKFEEAVEKFRSILLSVPLLVVDNKQEIAEAQQLITICREYIVGLSVETERKKLPKETLEQQKRICEMAAYFTHSNLQPVHMILVLRTALNLFFKLKNFKTAATFARRLLELGPKPEVAQQTRKILSACEKNPTDAYQLNYDMHNPFDICAASYRPIYRGKPVEKCPLSGACYSPEFKGQICRVTTVTEIGKDVIGLRISPLQFR

SEQ ID NO: 16 - protein, 1224 aa, Homo sapiens; identical to SEQ ID NO: 15
  ref <308>: DATABASE ACCESSION NUMBER: NCBI/NP_004362
  ref <309>: DATABASE ENTRY DATE: 2008-05-11
  ref <313>: RELEVANT RESIDUES IN SEQ ID NO: (1)..(1224)

SEQ ID NO: 17 - protein, 1233 aa, Homo sapiens
  ref <308>: DATABASE ACCESSION NUMBER: NCBI/NP_001091868
  ref <309>: DATABASE ENTRY DATE: 2008-05-11
  ref <313>: RELEVANT RESIDUES IN SEQ ID NO: (1)..(1233)
MLTKFETKSARVKGLSFHPKRPWILTSLHNGVIQLWDYRMCTLIDKFDEHDGPVRGIDFHKQQPLFVSGGDDYKIKVWNYKLRRCLFTLLGHLDYIRTTFFHHEYPWILSASDDQTIRVWNWQSRTCVCVLTGHNHYVMCAQFHPTEDLVVSASLDQTVRVWDISGLRKKNLSPGAVESDVRGITGVDLFGTTDAVVKHVLEGHDRGVNWAAFHPTMPLIVSGADDRQVKIWRMNESKAWEVDTCRGHYNNVSCAVFHPRQELILSNSEDKSIRVWDMSKRTGVQTFRRDHDRFWVLAAHPNLNLFAAGHDGGMIVFKLERERPAYAVHGNMLHYVKDRFLRQLDFNSSKDVAVMQLRSGSKFPVFNMSYNPAENAVLLCTRASNLENSTYDLYTIPKDADSQNPDAPEGKRSSGLTAVWVARNRFAVLDRMHSLLIKNLKNEITKKVQVPNCDEIFYAGTGNLLLRDADSITLFDVQQKRTLASVKISKVKYVIWSADMSHVALLAKHEHSCPLPLTAIVICNRKLDALCNIHENIRVKSGAWDESGVFIYTTSNHIKYAVTTGDHGIIRTLDLPIYVTRVKGNNVYCLDRECRPRVLTIDPTEFKFKLALINRKYDEVLHMVRNAKLVGQSIIAYLQKKGYPEVALHFVKDEKTRFSLALECGNIEIALEAAKALDDKNCWEKLGEVALLQGNHQIVEMCYQRTKNFDKLSFLYLITGNLEKLRKMMKIAEIRKDMSGHYQNALYLGDVSERVRILKNCGQKSLAYLTAATHGLDEEAESLKETFDPEKETIPDIDPNAKLLQPPAPIMPLDTNWPLLTVSKGFFEGTIASKGKGGALAADIDIDTVGTEGWGEDAELQLDEDGFVEATEGLGDDALGKGQEEGGGWDVEEDLELPPELDISPGAAGGAEDGFFVPPTKGTSPTQIWCNNSQLPVDHILAGSFETAMRLLHDQVGVIQFGPYKQLFLQTYARGRTTYQALPCLPSMYGYPNRNWKDAGLKNGVPAVGLKLNDLIQRLQLCYQLTTVGKFEEAVEKFRSILLSVPLLVVDNKQEIAEAQQLITICREYIVGLSVETERKKLPKETLEQQKRICEMAAYFTHSNLQPVHMILVLRTALNLFFKLKNFKTAATFARRLLELGPKPEVAQQTRKILSACEKNPTDAYQLNYDMHNPFDICAASYRPIYRGKPVEKCPLSGACYSPEFKGQICRVTTVTEIGKDVIGLRISPLQFR

SEQ ID NO: 18 - protein, 953 aa, Homo sapiens
  ref <308>: DATABASE ACCESSION NUMBER: UniProtKB/P53618
  ref <309>: DATABASE ENTRY DATE: 2009-05-05
  ref <313>: RELEVANT RESIDUES IN SEQ ID NO: (1)..(953)
MTAAENVCYTLINVPMDSEPPSEISLKNDLEKGDVKSKTEALKKVIIMILNGEKLPGLLMTIIRFVLPLQDHTIKKLLLVFWEIVPKTTPDGRLLHEMILVCDAYRKDLQHPNEFIRGSTLRFLCKLKEAELLEPLMPAIRACLEHRHSYVRRNAVLAIYTIYRNFEHLIPDAPELIHDFLVNEKDASCKRNAFMMLIHADQDRALDYLSTCIDQVQTFGDILQLVIVELIYKVCHANPSERARFIRCIYNLLQSSSPAVKYEAAGTLVTLSSAPTAIKAAAQCYIDLIIKESDNNVKLIVLDRLIELKEHPAHERVLQDLVMDILRVLSTPDLEVRKKTLQLALDLVSSRNVEELVIVLKKEVIKTNNVSEHEDTDKYRQLLVRTLHSCSVRFPDMAANVIPVLMEFLSDNNEAAAADVLEFVREAIQRFDNLRMLIVEKMLEVFHAIKSVKIYRGALWILGEYCSTKEDIQSVMTEIRRSLGEIPIVESEIKKEAGELKPEEEITVGPVQKLVTEMGTYATQSALSSSRPTKKEEDRPPLRGFLLDGDFFVAASLATTLTKIALRYVALVQEKKKQNSFVAEAMLLMATILHLGKSSLPKKPITDDDVDRISLCLKVLSECSPLMNDIFNKECRQSLSHMLSAKLEEEKLSQKKESEKRNVTVQPDDPISFMQLTAKNEMNCKEDQFQLSLLAAMGNTQRKEAADPLASKLNKVTQLTGFSDPVYAEAYVHVNQYDIVLDVLVVNQTSDTLQNCTLELATLGDLKLVEKPSPLTLAPHDFANIKANVKVASTENGIIFGNIVYDVSGAASDRNCVVLSDIHIDIMDYIQPATCTDAEFRQMWAEFEWENKVTVNTNMVDLNDYLQHILKSTNMKCLTPEKALSGYCGFMAANLYARSIFGEDALANVSIEKPIHQGPDAAVTGHIRIRAKSQGMALSLGDKINLSQKKTSI

SEQ ID NO: 19 - protein, 906 aa, Homo sapiens
  ref <308>: DATABASE ACCESSION NUMBER: UniProtKB/P35606
  ref <309>: DATABASE ENTRY DATE: 2009-05-05
  ref <313>: RELEVANT RESIDUES IN SEQ ID NO: (1)..(906)
MPLRLDIKRKLTARSDRVKSVDLHPTEPWMLASLYNGSVCVWNHETQTLVKTFEVCDLPVRAAKFVARKNWVVTGADDMQIRVFNYNTLERVHMFEAHSDYIRCIAVHPTQPFILTSSDDMLIKLWDWDKKWSCSQVFEGHTHYVMQIVINPKDNNQFASASLDRTIKVWQLGSSSPNFTLEGHEKGVNCIDYYSGGDKPYLISGADDRLVKIWDYQNKTCVQTLEGHAQNVSCASFHPELPIIITGSEDGTVRIWHSSTYRLESTLNYGMERVWCVASLRGSNNVALGYDEGSIIVKLGREEPAMSMDANGKIIWAKHSEVQQANLKAMGDAEIKDGERLPLAVKDMGSCEIYPQTIQHNPNGRFVVVCGDGEYIIYTAMALRNKSFGSAQEFAWAHDSSEYAIRESNSIVKIFKNFKEKKSFKPDFGAESIYGGFLLGVRSVNGLAFYDWDNTELIRRIEIQPKHIFWSDSGELVCIATEESFFILKYLSEKVLAAQETHEGVTEDGIEDAFEVLGEIQEIVKTGLWVGDCFIYTSSVNRLNYYVGGEIVTIAHLDRTMYLLGYIPKDNRLYLGDKELNIISYSLLVSVLEYQTAVMRRDFSMADKVLPTIPKEQRTRVAHFLEKQGFKQQALTVSTDPEHRFELALQLGELKIAYQLAVEAESEQKWKQLAELAISKCQFGLAQECLHHAQDYGGLLLLATASGNANMVNKLAEGAERDGKNNVAFMSYFLQGKVDACLELLIRTGRLPEAAFLARTYLPSQVSRVVKLWRENLSKVNQKAAESLADPTEYENLFPGLKEAFVVEEWVKETHADLWPAKQYPLVTPNEERNVMEEGKDFQPSRSTAQQELDGKPASPTPVIVASHTANKEEKSLLELEVDLDNLELEDIDTTDINLDEDILDD

SEQ ID NO: 20 - protein, 877 aa, Homo sapiens
  ref <308>: DATABASE ACCESSION NUMBER: NCBI/EAW79040
  ref <309>: DATABASE ENTRY DATE: 2006-12-18
  ref <313>: RELEVANT RESIDUES IN SEQ ID NO: (1)..(877)
MLASLYNGSVCVWNHETQTLVKTFEVCDLPVRAAKFVARKNWVVTGADDMQIRVFNYNTLERVHMFEAHSDYIRCIAVHPTQPFILTSSDDMLIKLWDWDKKWSCSQVFEGHTHYVMQIVINPKDNNQFASASLDRTIKVWQLGSSSPNFTLEGHEKGVNCIDYYSGGDKPYLISGADDRLVKIWDYQNKTCVQTLEGHAQNVSCASFHPELPIIITGSEDGTVRIWHSSTYRLESTLNYGMERVWCVASLRGSNNVALGYDEGSIIVKLGREEPAMSMDANGKIIWAKHSEVQQANLKAMGDAEIKDGERLPLAVKDMGSCEIYPQTIQHNPNGRFVVVCGDGEYIIYTAMALRNKSFGSAQEFAWAHDSSEYAIRESNSIVKIFKNFKEKKSFKPDFGAESIYGGFLLGVRSVNGLAFYDWDNTELIRRIEIQPKHIFWSDSGELVCIATEESFFILKYLSEKVLAAQETHEGVTEDGIEDAFEVLGEIQEIVKTGLWVGDCFIYTSSVNRLNYYVGGEIVTIAHLDRTMYLLGYIPKDNRLYLGDKELNIISYSLLVSVLEYQTAVMRRDFSMADKVLPTIPKEQRTRVAHFLEKQGFKQQALTVSTDPEHRFELALQLGELKIAYQLAVEAESEQKWKQLAELAISKCQFGLAQECLHHAQDYGGLLLLATASGNANMVNKLAEGAERDGKNNVAFMSYFLQGKVDACLELLIRTGRLPEAAFLARTYLPSQVSRVVKLWRENLSKVNQKAAESLADPTEYENLFPGLKEAFVVEEWVKETHADLWPAKQYPLVTPNEERNVMEEGKDFQPSRSTAQQELDGKPASPTPVIVASHTANKEEKSLLELEVDLDNLELEDIDTTDINLDEDILDD

SEQ ID NO: 21 - protein, 511 aa, Homo sapiens
  ref <308>: DATABASE ACCESSION NUMBER: UniProtKB/ P48444
  ref <309>: DATABASE ENTRY DATE: 2009-05-26
  ref <313>: RELEVANT RESIDUES IN SEQ ID NO: (1)..(511)
MVLLAAAVCTKAGKAIVSRQFVEMTRTRIEGLLAAFPKLMNTGKQHTFVETESVRYVYQPMEKLYMVLITTKNSNILEDLETLRLFSRVIPEYCRALEENEISEHCFDLIFAFDEIVALGYRENVNLAQIRTFTEMDSHEEKVFRAVRETQEREAKAEMRRKAKELQQARRDAERQGKKAPGFGGFGSSAVSGGSTAAMITETIIETDKPKVAPAPARPSGPSKALKLGAKGKEVDNFVDKLKSEGETIMSSSMGKRTSEATKMHAPPINMESVHMKIEEKITLTCGRDGGLQNMELHGMIMLRISDDKYGRIRLHVENEDKKGVQLQTHPNVDKKLFTAESLIGLKNPEKSFPVNSDVGVLKWRLQTTEESFIPLTINCWPSESGNGCDVNIEYELQEDNLELNDVVITIPLPSGVGAPVIGEIDGEYRHDSRRNTLEWCLPVIDAKNKSGSLEFSIAGQPNDFFPVQVSFVSKKNYCNIQVTKVTQVDGNSPVRFSTETTFLVDKYEIL

SEQ ID NO: 22 - protein, 552 aa, Homo sapiens
  ref <308>: DATABASE ACCESSION NUMBER: NCBI/ACA05944
  ref <309>: DATABASE ENTRY DATE: 2008-02-20
  ref <313>: RELEVANT RESIDUES IN SEQ ID NO: (1)..(552)
MAECNLVAILISSIDNPLDKNLDNGGNSCLDFRPLNSFSQPQVLLAAAVCTKAGKAIVSRQFVEMTRTRIEGLLAAFPKLMNTGKQHTFVETESVRYVYQPMEKLYMVLITTKNSNILEDLETLRLFSRVIPEYCRALEENEISEHCFDLIFAFDEIVALGYRENVNLAQIRTFTEMDSHEEKVFRAVRETQEREAKAEMRRKAKELQQARRDAERQGKKAPGFGGFGSSAVSGGSTAAMITETIIETDKPKVAPAPARPSGPSKALKLGAKGKEVDNFVDKLKSEGETIMSSSMGKRTSEATKMHAPPINMESVHMKIEEKITLTCGRDGGLQNMELHGMIMLRISDDKYGRIRLHVENEDKKGVQLQTHPNVDKKLFTAESLIGLKNPEKSFPVNSDVGVLKWRLQTTEESFIPLTINCWPSESGNGCDVNIEYELQEDNLELNDVVITIPLPSGVGAPVIGEIDGEYRHDSRRNTLEWCLPVIDAKNKSGSLEFSIAGQPNDFFPVQVSFVSKKNYCNIQVTKVTQVDGNSPVRFSTETTFLVDKYEIL

SEQ ID NO: 23 - protein, 308 aa, Homo sapiens
  ref <308>: DATABASE ACCESSION NUMBER: UniProtKB/O14579
  ref <309>: DATABASE ENTRY DATE: 2009-05-05
  ref <313>: RELEVANT RESIDUES IN SEQ ID NO: (1)..(308)
MAPPAPGPASGGSGEVDELFDVKNAFYIGSYQQCINEAQRVKLSSPERDVERDVFLYRAYLAQRKFGVVLDEIKPSSAPELQAVRMFADYLAHESRRDSIVAELDREMSRSVDVTNTTFLLMAASIYLHDQNPDAALRALHQGDSLECTAMTVQILLKLDRLDLARKELKRMQDLDEDATLTQLATAWVSLATGGEKLQDAYYIFQEMADKCSPTLLLLNGQAACHMAQGRWEAAEGLLQEALDKDSGYPETLVNLIVLSQHLGKPPEVTNRYLSQLKDAHRSHPFIKEYQAKENDFDRLVLQYAPSA

SEQ ID NO: 24 - protein, 256 aa, Homo sapiens
  ref <308>: DATABASE ACCESSION NUMBER: NCBI/NP_955476
  ref <309>: DATABASE ENTRY DATE: 2008-09-28
  ref <313>: RELEVANT RESIDUES IN SEQ ID NO: (1)..(256)
MAPPAPGPASGGSGEVDELFDVKNAFYIGSYQQCINEAQRVKLSSPERDVERDVFLYRAYLAQRKFGVVLDEIKPSSAPELQAVRMFADYLAHESRRDSIVAELDREMSRSVDVTNTTFLLMAASIYLHDQNPDAALRALHQGDSLECTAMTVQILLKLDRLDLARKELKRMQDLDEDATLTQLATAWVSLATDSGYPETLVNLIVLSQHLGKPPEVTNRYLSQLKDAHRSHPFIKEYQAKENDFDRLVLQYAPSA

SEQ ID NO: 25 - protein, 257 aa, Homo sapiens
  ref <308>: DATABASE ACCESSION NUMBER: NCBI/NP_955474
  ref <309>: DATABASE ENTRY DATE: 2008-09-28
  ref <313>: RELEVANT RESIDUES IN SEQ ID NO: (1)..(257)
MAPPAPGPASGGSGEVDELFDVKNAFYIGSYQQCINEAQRVKLSSPERDVERDVFLYRAYLAQRKFGVVLDEIKPSSAPELQAVRMFADYLAHESRSTAMTVQILLKLDRLDLARKELKRMQDLDEDATLTQLATAWVSLATGGEKLQDAYYIFQEMADKCSPTLLLLNGQAACHMAQGRWEAAEGLLQEALDKDSGYPETLVNLIVLSQHLGKPPEVTNRYLSQLKDAHRSHPFIKEYQAKENDFDRLVLQYAPSA

SEQ ID NO: 26 - protein, 874 aa, Homo sapiens
  ref <308>: DATABASE ACCESSION NUMBER: UniProtKB/ Q9Y678
  ref <309>: DATABASE ENTRY DATE: 2009-04-14
  ref <313>: RELEVANT RESIDUES IN SEQ ID NO: (1)..(874)
MLKKFDKKDEESGGGSNPFQHLEKSAVLQEARVFNETPINPRKCAHILTKILYLINQGEHLGTTEATEAFFAMTKLFQSNDPTLRRMCYLTIKEMSCIAEDVIIVTSSLTKDMTGKEDNYRGPAVRALCQITDSTMLQAIERYMKQAIVDKVPSVSSSALVSSLHLLKCSFDVVKRWVNEAQEAASSDNIMVQYHALGLLYHVRKNDRLAVNKMISKVTRHGLKSPFAYCMMIRVASKQLEEEDGSRDSPLFDFIESCLRNKHEMVVYEAASAIVNLPGCSAKELAPAVSVLQLFCSSPKAALRYAAVRTLNKVAMKHPSAVTACNLDLENLVTDSNRSIATLAITTLLKTGSESSIDRLMKQISSFMSEISDEFKVVVVQAISALCQKYPRKHAVLMNFLFTMLREEGGFEYKRAIVDCIISIIEENSESKETGLSHLCEFIEDCEFTVLATRILHLLGQEGPKTTNPSKYIRFIYNRVVLEHEEVRAGAVSALAKFGAQNEEMLPSILVLLKRCVMDDDNEVRDRATFYLNVLEQKQKALNAGYILNGLTVSIPGLERALQQYTLEPSEKPFDLKSVPLATAPMAEQRTESTPITAVKQPEKVAATRQEIFQEQLAAVPEFRGLGPLFKSSPEPVALTESETEYVIRCTKHTFTNHMVFQFDCTNTLNDQTLENVTVQMEPTEAYEVLCYVPARSLPYNQPGTCYTLVALPKEDPTAVACTFSCMMKFTVKDCDPTTGETDDEGYEDEYVLEDLEVTVADHIQKVMKLNFEAAWDEVGDEFEKEETFTLSTIKTLEEAVGNIVKFLGMHPCERSDKVPDNKNTHTLLLAGVFRGGHDILVRSRLLLLDTVTMQVTARSLEELPVDIILASVG

SEQ ID NO: 27 - protein, 871 aa, Homo sapiens
  ref <308>: DATABASE ACCESSION NUMBER: NCBI/NP_036265
  ref <309>: DATABASE ENTRY DATE: 2008-08-20
  ref <313>: RELEVANT RESIDUES IN SEQ ID NO: (1)..(871)
MIKKFDKKDEESGSGSNPFQHLEKSAVLQEARIFNETPINPRRCLHILTKILYLLNQGEHFGTTEATEAFFAMTRLFQSNDQTLRRMCYLTIKEMATISEDVIIVTSSLTKDMTGKEDVYRGPAIRALCRITDGTMLQAIERYMKQAIVDKVSSVSSSALVSSLHMMKISYDVVKRWINEAQEAASSDNIMVQYHALGVLYHLRKNDRLAVSKMLNKFTKSGLKSQFAYCMLIRIASRLLKETEDGHESPLFDFIESCLRNKHEMVIYEAASAIIHLPNCTARELAPAVSVLQLFCSSPKPALRYAAVRTLNKVAMKHPSAVTACNLDLENLITDSNRSIATLAITTLLKTGSESSVDRLMKQISSFVSEISDEFKVVVVQAISALCQKYPRKHSVMMTFLSNMLRDDGGFEYKRAIVDCIISIVEENPESKEAGLAHLCEFIEDCEHTVLATKILHLLGKEGPRTPVPSKYIRFIFNRVVLENEAVRAAAVSALAKFGAQNESLLPSILVLLQRCMMDTDDEVRDRATFYLNVLQQRQMALNATYIFNGLTVSVPGMEKALHQYTLEPSEKPFDMKSIPLAMAPVFEQKAEITLVATKPEKLAPSRQDIFQEQLAAIPEFLNIGPLFKSSEPVQLTEAETEYFVRCIKHMFTNHIVFQFDCTNTLNDQLLEKVTVQMEPSDSYEVLSCIPAPSLPYNQPGICYTLVRLPDDDPTAVAGSFSCTMKFTVRDCDPNTGVPDEDGYDDEYVLEDLEVTVSDHIQKVLKPNFAAAWEEVGDTFEKEETFALSSTKTLEEAVNNIITFLGMQPCERSDKVPENKNSHSLYLAGIFRGGYDLLVRSRLALADGVTMQVTVRSKERTPVDVILASVG

SEQ ID NO: 28 - protein, 768 aa, Homo sapiens [sequence omitted]

SEQ ID NO: 29 - protein, 177 aa, Homo sapiens
  ref <308>: DATABASE ACCESSION NUMBER: UniProtKB/P61923
  ref <309>: DATABASE ENTRY DATE: 2009-05-05
  ref <313>: RELEVANT RESIDUES IN SEQ ID NO: (1)..(177)
MEALILEPSLYTVKAILILDNDGDRLFAKYYDDTYPSVKEQKAFEKNIFNKTHRTDSEIALLEGLTVVYKSSIDLYFYVIGSSYENELMLMAVLNCLFDSLSQMLRKNVEKRALLENMEGLFLAVDEIVDGGVILESDPQQVVHRVALRGEDVPLTEQTVSQVLQSAKEQIKWSLLR

SEQ ID NO: 30 - protein, 210 aa, Homo sapiens
  ref <308>: DATABASE ACCESSION NUMBER: NCBI/NP_057513
  ref <309>: DATABASE ENTRY DATE: 2009-03-29
  ref <313>: RELEVANT RESIDUES IN SEQ ID NO: (1)..(210)
MQRPEAWPRPHPGEGAAAAQAGGPAPPARAGEPSGLRLQEPSLYTIKAVFILDNDGRRLLAKYYDDTFPSMKEQMVFEKNVFNKTSRTESEIAFFGGMTIVYKNSIDLFLYVVGSSYENELMLMSVLTCLFESLNHMLRKNVEKRWLLENMDGAFLVLDEIVDGGVILESDPQQVIQKVNFRADDGGLTEQSVAQVLQSAKEQIKWSLLK

(19) United States
(12) Patent Application Publication
(10) Pub. No.: US 2017/0021040 A1
(43) Pub. Date: Jan. 26, 2017

(54) PLASMON ENERGY THERAPEUTICS

(71) Applicants: FRANCO VITALIANO, BOSTON, MA (US); GORDANA DRAGAN VITALIANO, BOSTON, MA (US)

(72) Inventors: FRANCO VITALIANO, BOSTON, MA (US); GORDANA DRAGAN VITALIANO, BOSTON, MA (US)

(21) Appl. No.: 15/289,147

(22) Filed: Oct. 8, 2016

Related U.S. Application Data
(62) Division of application No. 13/443,216, filed on Apr. 10, 2012, now abandoned.

(51) Int. Cl. A61K 49/00; A61B 5/00; A61M 31/00; A61N 1/44; A61F 2/82; G01N 21/64; A61B 18/04

(52) U.S. Cl. CPC A61K 49/005; G01N 21/648; A61B 5/0071; A61B 18/042; A61N 1/44; A61F 2/82; A61M 31/002; A61F 2007/0089

(57) ABSTRACT

The invention in suitable embodiments is directed to plasmon energy therapeutics. In one embodiment the instant invention is a medicament composition in which manmade nanoscale elements, such as ultrabright fluorescent molecules and emitting photons of a luminance equivalent to at least a luminance of photons emitted by a quantum dot, are disposed adjacent to dielectric elements and further including multiple metal molecules capable of photo-accepting the emitted photons. In one aspect, the composition produces chemical, electrical, electromagnetic, optical, plasmonic, or thermal energy. In one embodiment, the invention is a drug for use in treating a disease, condition, or disorder. In another embodiment, energy is produced, captured or stored or directed or transduced by the composition for use by a cell.

### FIELD OF THE INVENTION

This application claims priority to Apr. 10, 2012, U.S. patent application Ser. No. 13/443,216, with the title, “BIO-NANO-PLASMONIC ELEMENTS AND PLATFORMS”. The invention relates generally to the field of plasmon energy therapeutics, and in one embodiment is a medicament composition in which manmade nanoscale elements, such as ultrabright fluorescent molecules and emitting photons of a luminance equivalent to at least a luminance of photons emitted by a quantum dot, are disposed adjacent to dielectric elements and further including multiple metal molecules capable of photo-accepting the emitted photons. In one aspect, the composition produces chemical, electrical, electromagnetic, optical, plasmonic, or thermal energy. In one embodiment, the invention is a drug for use in treating a disease, condition, or disorder. In another embodiment, one or more type of energy is produced, captured or stored or directed or transduced by the composition for use by a cell.

The invention relates generally to the field of plasmonics, and more specifically, in one embodiment, to one or more nanoscale plasmonic elements that have one or more properties, aspects, and or functions, and formed in whole or in part from one or more self-assembling protein molecules.

In one embodiment of the present invention, it relates to at least one or more nanoscale elements that emit one or more types of surface-plasmon-enhanced electromagnetic radiation, including any energies, and to methods of fabricating these elements in whole or in part using one or more self-assembling elements comprised of purified, synthetic and or recombinant protein molecule elements and or their accessory elements, and in particular, composed of at least one or more Clathrin and or Coatomer I/II protein molecules, forming one or more elements of one or more molecular weights, dimensions, geometries, symmetries, configurations and combinations.

In one embodiment of the present invention it allows for confining light in dimensions smaller than the wavelength of photons in free space, and makes it possible to match the different length scales associated with photonics and electronics in a single nanoscale device.

In another embodiment, the invention relates to a plasmonic platform and its associated one or more types of electromagnetic radiation, including any energy aspects, such as a biomedical platform, telecommunication platform, environmental remediation platform, computational platform, and the like, using such elements

### BACKGROUND OF THE INVENTION

Metals can absorb light by creating plasmons, which are particle-like collective excitations of conduction electrons at a metallic surface. Surface Plasmon Resonance is thus usually characterised as a quantum optical-electrical phenomenon arising from the interaction of light with a metal surface. This phenomenon occurring at metal/dielectric interfaces has the further aspect of using such excitations for the localization of electromagnetic radiation, including any energy aspects in one or more dimensions, configurations and combinations, which may be utilized in a broad range of applications.

Under normal circumstances, a laser light source incident upon a thin film is reflected or scattered, resulting in insignificant excitation of surface plasmon waves, which absorb only very little of the incident energy. At certain specific wavelengths of the laser source, however, plasmon resonance phenomena occur.

In one embodiment, conductive electrons in metal film, excited at such wavelengths, oscillate in phase with the incident energy, and strongly enhance the electromagnetic field at the interface between the film and insulator media, producing surface plasmon waves with large amplitude. An increase in the spontaneous emission rate of light is a telltale sign of this altered interaction; in one prior art study, researchers measured a six-fold increase in the spontaneous emission rate of light in a gap size of 5 nanometers. A plasmonic bio-nanoparticle embodiment could be made as small as one nanometer, but any smaller than that, and the bio-nanoparticle's functionality breaks down.

Plasmons are transient entities: they typically live for mere attoseconds, and cannot travel more than a few plasmon wavelengths in a metal before their energy is absorbed by the great amount of non-oscillating electrons around them. For some period of time, it was not at all clear how plasmons could be made to amplify.

Usually, plasmons are absorbed by the metal almost as soon as they are produced. The challenge was to make sure this energy does not dissipate rapidly from the metal surface. In one embodiment, the light bounces around on the surface of a noble, alkali, and or other suitable metal sphere in the nanoparticle's core in the form of plasmons.

A plasmonic element must be “pumped” to supply the necessary energy. This pumping is accomplished by bombarding the bio-nanoparticle with pulses of light. In one embodiment, light from an invention element can remain confined as plasmons or it can be made to leave the particle surface as photons in the visible-light range.

In more detail, under certain conditions the energy carried by photons of light is transferred to packets of electrons, called plasmons, on a metal's surface. Energy transfer occurs only at a specific resonance wavelength of light. That is, the wavelength where the quantum energy carried by the photons exactly equals the quantum energy level of the plasmons. The plasmon state is a highly delocalized state formed collectively through Coulombian (electrostatic) interaction of weakly bound electrons. Surface plasmon polaritons (SPP), charge density oscillations at metal-dielectric interfaces, have shown great utility and significant future potential in a number of application areas. For example, they have been proven to provide an excellent means to probe biochemical events due to their strong local field enhancement near metal surfaces or nanostructures. Under specific conditions, the incident light couples with the surface plasmons to create self-sustaining, propagating electromagnetic waves known as surface plasmon polaritons, or SPPs. Thus, a polariton is the result of the mixing of a photon with an excitation of a material.

One or more types of metals can be used for enabling plasmonic elements. For example, noble metals demonstrate shorter plasmon wavelengths than transition metals. Both noble (Au, Ag, Cu) and alkali metals demonstrate strong plasmon resonances. For example, gold red shifts below 25 nm and blue shifts above 25 nm in size.

The noble metals are metals that are resistant to corrosion and oxidation in moist air, unlike most base metals. They tend to be precious, often due to their rarity in the Earth's crust. The noble metals are considered to be (in order of increasing atomic number) ruthenium, rhodium, palladium, silver, osmium, iridium, platinum, and gold. Other sources include mercury or even rhenium. On the other hand, titanium, niobium and tantalum are not included as noble metals despite the fact that they are very resistant to corrosion. Alkali metals on the other hand comprise any of a group of soft, white, low-density, low-melting, highly reactive metallic elements, including lithium, sodium, potassium, rubidium, cesium, and francium.

In addition, metamaterials, tailor made composites, can comprise one or more current invention elements. Metamaterials are artificial structures composed of tailored sub-wavelength building blocks. Metamaterials are found able to greatly improve the capabilities to manipulate electromagnetic radiation almost throughout the entire spectrum, providing many intriguing properties and phenomena, such as negative refractive index n, superlensing, and invisibility. Plasmonic metamaterials based on metal-dielectric nanostructures exhibit unique optical properties and find numerous applications in such diverse areas as optical communications, chemo- and bio-sensing, high-density data storage and many others. These properties can be tailored to specific applications and requirements by changing structural and dielectric parameters of the constituents. Negative index metamaterials thus exploit surface plasmons produced from the interaction of light with metal-dielectric materials. Also known as left-handed or negative index materials, metamaterials exhibit optical properties opposite to those of glass or air. These have been termed positive index—materials of our everyday world. In particular, energy is transported in a direction opposite to that of propagating wavefronts, rather than traveling in lockstep, as is the case in positive index materials. As a result, when juxtaposed with a positive index material, negative index materials exhibit counterintuitive properties, like bending, or refracting, light in unnatural ways.

Multiple detection schemes have already been developed and commercialized to experimentally characterize molecular binding events. The field of plasmonics has also produced novel laser “spasers”, as well as enabling new SPP applications for drug delivery and the destruction of cancer cells. Computing and communications are also strong application candidates for plasmonic devices. Many of these applications make use of large external light sources and detectors. But by combining integrated SPP sources with previously designed SPP detectors, and or with new nanoscale source and detector variants together with novel bio-nanoscale elements, the current invention integrates plasmonics with a new class of bio-nanotechnology elements and platforms. In doing so, the instant invention paves the way for numerous new types of bio-nanotechnology-based applications, going from integrated plasmonic bio-nano-devices, to arrays of small-scale, to fully integrated plasmonic nano-biosensors, to bio-nanolasers, and to biomedical applications, to just to name some, and many of which applications can operate both in vitro and in vivo. These new bio-nano-plasmonic elements and platforms and their associated one or more types of electromagnetic radiation, including any energy aspects can be used in a wide variety of embodiments and settings, from medical, to industrial, to commercial, and represent a significant improvement in the art, and are described herein.

In the instant invention, in in one or more embodiments, we present the novel design of an integrated bio-nanotechnology source of strongly confined SPPs in one or more types of waveguides, including, but not limited to, insulator dielectric, plasmon slot, metal-insulator-metal, other types of metallic slot waveguides, ring shaped, and various other waveguide configurations. SPP generation has been demonstrated in the art using organic LEDs and silicon nanocrystals, among other types, and the current invention can also utilize these techniques in various novel embodiments. And the current invention further improves on the art by also showing novel types of SPP generation.

In general, the instant invention improves upon a number of technical aspects known in the art by utilizing a novel bio-nano-plasmonic element. As background to the current application, the field of plasmonics is herein summarized and discussed. The field of plasmonics was perhaps best summed up by Atwater (2007); whose group at the Applied Physics and Materials Science department at the California Institute of Technology first gave the field its name; who said, the use of metallic structures to transmit light signals seems impractical, because metals are known for high optical losses. The electrons oscillating in the electro-magnetic field collide with the surrounding lattice of atoms, rapidly dissipating the field's energy. But the plasmon losses are lower at the interface between a thin metal film and a dielectric (a non-conductive element like air or glass) than inside the bulk of a metal because the field spreads into the nonconductive material, where there are no free electrons to oscillate and hence no energy-dissipating collisions. This property naturally confines plasmons to the metallic surface abutting the dielectric; in a sandwich with dielectric and metal layers, for example, the surface plasmons propagate only in the thin plane at the interface. In other words, these planar plasmonic structures act as wave-guides, shepherding the electromagnetic waves along the metal-dielectric boundary. Thus, surface plasmons (SPs) are electron oscillations that allow electromagnetic radiation to be localized, confined, and guided on sub-wavelength scales.

Surface plasmon waves are therefore charge density waves occurring at an interface between a thin metallic film and an insulator medium, a dielectric. This phenomenon is the basis of many standard tools for measuring adsorption of material onto planar metal (noble, alkali, and or other suitable metals) surfaces or onto the surface of metal nanoparticles. It is the basis behind many color based biosensor applications and different lab-on-a-chip sensors.

The excitation of surface plasmons by light is denoted as surface plasmon resonance (SPR) for planar surfaces or localized surface plasmon resonance (LSPR) for nanometer-sized metallic structures. In the macro-world, the electromagnetic field does not penetrate inside the metal (in other words, the skin depth is much smaller than the characteristic size of the metal structure). Therefore, surface waves can be excited on a surface of a macro-object but decay rapidly as they propagate along the surface. In the opposite case of thin metal films, surface plasmon waves live long enough to be registered.

The wave can be thought of as having a section in the thin film and a section out of the film, at the insulator/metal interface, much like an ocean wave has part of the wave unseen inside the ocean, while another part of the wave is seen at the ocean/horizon interface. The surface electromagnetic waves propagate in a direction parallel to the metal/dielectric interface. Since the wave is on the boundary of the metal and the external medium (air or water for example), these oscillations are very sensitive to any change of this boundary, such as the adsorption of molecules to the metal surface. In one or more embodiments, in order to excite surface plasmons in a resonant manner, an electron or light beam (visible and infrared are typical) are used.

In one embodiment, suitably engineered metal nanostructures using one or more methods known in the art can also act as antennas in which the resonant coupling between the particles concentrates light into well-defined hot spots enabling ultrasmall, wavelength-sensitive directional sensors or detectors. In another embodiment, the same metal particle arrays, when coupled to optical emitters, can also act as directional emitters. In another embodiment, the enhanced optical density of states near the surface of metal nanoparticles can provide control over the color, directionality, and polarization of light-emitting diodes; which, in one embodiment can find large-scale applications in the area of photovoltaics. In such a photovoltaics embodiment, light scattering from metal nanoparticle arrays placed on top of a thin-film semiconductor layer can effectively fold the path of sunlight into the layer, strongly enhancing its effective absorption.

Parallel to the development of plasmonic structures based on metal nanoparticles, the propagation of plasmons along various types of waveguides are also known in the art. In one embodiment, precise control over material and geometry allows the wave-guiding properties to be controlled in ways that cannot be achieved with regular dielectric waveguides. In one example embodiment, absorption losses can be minimized by turning plasmonic waveguides inside out, putting the dielectric at the core and surrounding it with metal. In this device embodiment, called a plasmon slot waveguide, adjusting the thickness of the dielectric core changes the wavelength of the plasmons. Other plasmon waveguide example embodiments include ring-shaped waveguides, as well as three-dimensional plasmonic slot waveguides, including mirrors, bends, T-splitters and X-junctions for on-chip systems. In another example embodiment, using an integrated light-emitting diode (LED) and sub-wavelength slits, it is possible to couple light emitted by the LED directly into waveguided plasmon modes. These metal-insulator-metal (MIM) waveguides can combine high spatial field confinement (and propagation lengths of several micrometers.) Also, since the metallic cladding layers ensure that the fields are confined within the waveguide, one can easily separate plasmonic effects from any other non-plasmonic background radiation.

The magnitude and the direction of a surface plasmon polariton (SPP) in a nanoscale plasmonic structure are essential to modern plasmonic technology. In particular, in one embodiment, extremely short wavelengths can be achieved at optical frequencies. It has been shown in prior art that light with a free-space wavelength of 651 nm, squeezed in a metal-insulator-metal plasmonic waveguide, has its wavelength shrunk to only 58 nm. In another embodiment, it may be possible to shrink it still further, into the soft x-ray wavelength regime. Similar to the coupling within nanoparticle assemblies, this effect is due to the coupling between plasmons propagating at the metal-insulator interfaces. By further tailoring plasmonic waveguide structures in one or more instances of the current invention, the propagation speed of plasmons can be reduced well below the speed of light. More complex geometries, as featured in other embodiment in which arrays of nano-holes are integrated in a metal film, the current invention acts as efficient color filters. In some embodiment, geometries, plasmon waveguides exhibit a negative refractive index for the guided plasmon. In prior art two-dimensional negative refraction has been observed in these plasmonic waveguides.

Various methods are reported in the art for tuning surface plasmon resonance (SPR), and are implemented in one or more instances of the current invention. For example, SPP generation from a single slit has been used in many applications for its efficient coupling ratio. In most applications, the direction of SPP's generated from a narrow slit cannot be selected by usual optical excitation because positive and negative propagating SPPs are equally excited in the slit by the polarized electromagnetic field. But Choi, et al, showed that SPP propagating direction could be controlled by adjusting the spatiotemporal phase between two femtosecond laser pulses. The time delay between the two pulses is an additional degree of freedom, which gives strong interference effect in the SPP generation and propagation.

There is also other work in the art for exploring the properties of metal shell elements used in some types of plasmonic elements in the art, and which are implemented in one or more invention embodiments. For example, Ye et al, 2009 reported that the sensitivity of plasmon blending and splitting to the surrounding refractive index might be improved by increasing the shell thickness, aspect ratio or core refractive index. This local refractive index dependent-plasmon blending and splitting presents a new sensing picture based on tuning the number of SPR absorption peaks. In one example embodiment, the SPR frequency is determined by the size, shape, structure, and local dielectric environment of metallic nanoparticles that implemented via one or more bioengineering methods known in the art. In one embodiment, tunable plasmonic properties present various new application potentials, from chemical and biologic sensing to cancer imaging, and thermal therapy applications.

In another embodiment, one or more symmetrical and or broken-symmetrical metal shell nanostructures composed of a noble metal, an alkali metal, other suitable metal are implemented using one or more bioengineering methods known in the art. In another embodiment, the optical properties of one or more types of closed or open plasmon shell elements are adjusted by using custom designed metamaterials comprised of one or more suitable elements.

It has been found that breaking the symmetry of metal shell nanostructures results in a number of new optical phenomena. For example, Ye, et al, showed that the fractional height dependent optical properties are mainly attributed to the symmetry-broken geometry and are explained well by the plasmon hybridization model. In one embodiment, open shell nanostructures comprise one or more types of biomolecular detection schemes.

For example, gold “nano-eggs” composed of a nano-core allow for an excitation mixing of dipolar components in all plasmon modes of the particles. Nanocup and nanocap arrays also show highly tunable optical properties, and they render their optical properties dependent on the angle and polarization of the incident light. Besides their unconventional optical properties arising from the broken symmetry of gold shell geometry, these asymmetrical nanostructures possess a common feature of an enhanced localized electric field (“hot spot”) with a suitable excitation light compared to the full gold shell nanostructures.

Evlyukhin, et al have also found that by tuning the incident angle of an external light beam and the parameters of the surface nanoparticle structures one could obtain symmetric or asymmetric excitation of surface plasmon polaritons (SPP) beams propagating into certain directions. The reasons and conditions for this behavior and the efficiency of SPP excitation is a function of the incident angle. One or more such methods as described by Evlyukhin are also feasible in one or more invention embodiments.

Biosensors based on the phenomenon of surface plasmon resonance comprising evanescent wave techniques are featured in another embodiment. This feature utilizes a property of gold and other materials; for example, a thin layer of gold on a high refractive index glass surface can absorb laser light, producing electron waves (surface plasmons) on a noble, alkali, and or other suitable metal surface. This occurs only at a specific angle and wavelength of incident light and is highly dependent on the surface of the gold, such that binding of a target analyte to a receptor on a noble, alkali, and or other suitable metal surface produces a measurable signal. The plasmon resonant scattering from a single noble, alkali, and or other suitable metal nanoparticle is many orders of magnitude brighter than the signal from single fluorophores, fluorescent beads or quantum dots in microscopic imaging applications. In addition, the scattering signal does not photobleach or blink and polarization reveals the local orientation for nonspherical nanoparticle.

Thus, some types of optical resonances featured in one embodiment are surface plasmon resonance, which results in anomalous reflection and high evanescent fields at resonance. This phenomenon is the basis of many standard tools for measuring adsorption of material onto planar metal (e.g., gold, silver) surfaces or onto the surface of metal nanoparticles. It is the fundamentals behind many color based biosensor applications and different lab-on-a-chip sensors. Surface plasmons have been used to enhance the surface sensitivity of several spectroscopic measurements including fluorescence, Raman scattering, and second harmonic generation. For nanoparticles, localized surface plasmon oscillations can give rise to the intense colors of suspensions or sols containing the current invention's nanoparticles.

In one embodiment, the current invention's bio-nanoparticles comprised of one or more types of metals exhibit strong absorption bands in the ultraviolet-visible light regime that are not present in the bulk metal. This extraordinary absorption increase has been exploited to increase light absorption in photovoltaic cells by depositing metal nanoparticles on the cell surface. The energy (color) of this absorption differs when the light is polarized along or perpendicular to the nanowire. Shifts in this resonance due to changes in the local index of refraction upon adsorption to the nanoparticles can also be used to detect biopolymers such as DNA or proteins. Related complementary techniques include plasmon waveguide resonance, QCM, extraordinary optical transmission, and Dual Polarization Interferometry. However, in their simplest form, SPR reflectivity measurements can be used to detect molecular adsorption, such as polymers, DNA or proteins, etc. Technically, it is common that the angle of the reflection minimum (absorption maximum) is measured. This angle changes in the order of 0.1° during thin (about nm thickness) film adsorption. In other cases the changes in the absorption wavelength is followed. The mechanism of detection in one embodiment is that the adsorbing molecules cause changes in the local index of refraction, changing the resonance conditions of the surface plasmon waves.

Plasmonic nanoparticle pairs known as “dimers” are important for understanding the dynamics of hybridized plasmons in complex nanostructures. Classical physics approaches show distinct behaviors in two different regimes: when the particles are close to each other but not touching, and when the particles are touching with conductive overlap. In the non-touching regime, the bonding dipolar dimer plasmon red shifts monotonically with decreasing inter-particle separation. However, when conductive overlap is established between the nanoparticles, a new plasmon mode is enabled. This is the charge transfer plasmon (CTP) and involves conduction electrons flowing back and forth between the two nanoparticles, and CTP a feature in one invention embodiment. Its resonant frequency has been observed to blue shift when increasing the overlap between the two nanoparticles. It has been found that for large inter-particle separation distances, quantum calculations agree with the predictions of the classical approach for both plasmon energy and field enhancement. However, for nanoparticle separations smaller than 1 nm, quantum mechanical effects begin to significantly influence the plasmonic response of the dimer. The major effect is the onset of electron tunneling between the two nanoparticles, resulting in significantly smaller hybridization and a strong reduction of the electromagnetic field enhancements across the junction. For separations smaller than 0.5 nm in one embodiment, a charge transfer plasmon appears which blue shifts with decreasing inter-particle separation.

Typically, different plasmonic elements for manipulating surface plasmons are realized either through structuring metal surfaces or by placing dielectric structures on metals. Both approaches are based on the fabrication of permanent nanostructures on the metal surface, which are very difficult if not impossible to reconfigure in real time. However, reconfigurability is crucial to optical interconnections, which in turn are crucial for high performance optical computing and communication systems.

In one or more embodiments, this real time configurability impediment is overcome by embodying one or more exemplar techniques like those described by Zhang, et al, 2011, of Berkeley Lab's Materials Sciences Division, University of California at Berkeley's Nano-scale Science and Engineering Center (SINAM), which permit light rays to travel without diffraction in a curved arc in free space. These rays of light are termed “Airy beams,” after the English astronomer Sir George Biddell Airy, who studied what appears to be the parabolic trajectory of light in a rainbow. In one embodiment, a plasmonic Airy beam can manipulate SPPs without the need of any waveguide structures over metallic surfaces, providing dynamic control of their ballistic trajectories despite any surface roughness and defects, or getting around obstacles. One invention embodiment can provide dynamic control in real-time of the curved trajectories of Airy beams over metallic surfaces. In one embodiment, a grating coupler element directly couples free-space Airy beams to surface plasmon polaritons (SPPs). By directly coupling Airy beams to SPPs, the current invention in one embodiment can manipulate light at an extremely small scale beyond the diffraction limit. In one embodiment, Airy beams enable reconfigurable optical interconnections and also precisely manipulating particles on extremely small scales.

The reconfigurability afforded by Airy beams enables a significant advantage over previous approaches. In another embodiment, the unique properties of plasmonic Airy beams provides for energy routing along arbitrary trajectories in plasmonic elements, and allows for dynamic manipulations of nano-particles on metal surfaces and in magneto-electronic devices. In one embodiment, dynamic control of plasmonic Airy beams is provided by a computer-controlled spatial light modulator, a device similar to a liquid crystal display that can be used to offset the incoming light waves from a laser beam with respect to a cubic phase system mask and a Fourier lens. This technique generates a plasmonic Airy beam on the surface of a metal whose ballistic motion can be modified. In one embodiment, the direction and speed of the displacement between the incoming light and the cubic phase mask can be controlled by displaying an animation of the shifting mask pattern as well as a shifting slit aperture in the spatial light modulator. Depending on the refresh rate of the spatial light modulator this can be done in real time. In another embodiment, a spatial light modulator not only sets the plasmonic Airy beam into a general ballistic motion, it also permits the control of an Airy beam's peak intensity at different positions along its curved path. In biology and chemistry embodiments, the Airy beam technique enables the ability to dynamically manipulate molecules.

In another embodiment, dynamic tunable plasmonic Airy beams can be employed for ultrahigh resolution bioimaging. In one example embodiment, the invention can directly illuminate a target, for example a protein, bypassing any obstacles or reducing the background.

In one embodiment, surface plasmon resonance sensors operate using one or more bio-nano-sensor chips encased within a plastic cassette apparatus that is supporting a glass plate, one side of which is coated with a microscopic layer of a noble, alkali, and or other suitable metal. This side contacts the optical detection apparatus of the instrument. The opposite side is then contacted with a microfluidic flow system. The contact with the flow system creates channels across which reagents can be passed in solution. This side of the glass sensor chip can be modified in a number of ways known in the art, to allow easy attachment of molecules of interest. Normally it is coated in carboxymethyl dextran or similar compound. Light of a fixed wavelength is reflected off the gold side of the chip at the angle of total internal reflection, and detected inside the instrument. This induces the evanescent wave to penetrate through the glass plate and some distance into the liquid flowing over the surface. The refractive index at the flow side of the chip surface has a direct influence on the behavior of the light reflected off the metal side. Binding to the flow side of the chip has an effect on the refractive index and in this way biological interactions can be measured to a high degree of sensitivity with some sort of energy. Another evanescent wave bio-nanosensor application embodiment uses waveguides, wherein the propagation constant through the waveguide is changed by the absorption of molecules to the waveguide surface. One such example in the prior art, Dual Polarization Interferometry, uses a buried waveguide as a reference against which the change in propagation constant is measured. Other configurations known in the art such as the Mach-Zehnder have reference arms lithographically defined on a substrate. Higher levels of integration can be achieved in other invention embodiment by using resonator geometries where the resonant frequency of a ring resonator changes when molecules are absorbed.

Other optical bio-nanosensor application embodiments are based on changes in absorbance or fluorescence of an appropriate indicator compound and do not need a total internal reflection geometry. For example, in the prior art a fully operational prototype device detecting casein in milk has been fabricated. The device is based on detecting changes in absorption of a gold layer. A widely used research tool, the micro-array, can also be considered a biosensor, one or more forms of which can be implemented in one or embodiments.

Some nanobiosensor applications in the art use an immobilized bioreceptor probe that is selective for target analyte molecules, and are also featured in one invention embodiment. Nanomaterials are exquisitely sensitive chemical and biological sensors. Nanoscale materials demonstrate unique properties. Their large surface area to volume ratio can achieve rapid and low cost reactions, using a variety of designs.

As in some prior art, one biological bio-nanosensor embodiment incorporates a genetically modified form of a native protein or enzyme. In this embodiment, the protein is configured to detect a specific analyte and a detection instrument such as a fluorometer or luminometer reads the ensuing signal. An example of a recently developed biosensor in the prior art is one for detecting cytosolic concentration of the analyte cAMP (cyclic adenosine monophosphate), a second messenger involved in cellular signaling triggered by ligands interacting with receptors on the cell membrane. Similar systems have been created to study cellular responses to native ligands or xenobiotics (toxins or small molecule inhibitors). Pharmaceutical and biotechnology companies commonly use such “assays” in drug discovery development. Most cAMP assays in current use require lysis of the cells prior to measurement of cAMP A live-cell biosensor for cAMP can be used in non-lysed cells with the additional advantage of multiple reads to study the kinetics of receptor response.

If the surface is patterned with different biopolymers, using adequate optics and imaging sensors (i.e. a camera), the technique can be extended in one embodiment to surface plasmon resonance imaging (SPRI). This method provides a high contrast of the images based on the adsorbed amount of molecules, somewhat similar to Brewster angle microscopy (this latter is most commonly used together with a Langmuir-Blodgett trough). Extinction, which can be tuned to the near infrared, further allows plasmonics-based nanoparticles to act as molecular contrast agents in a spectral region where tissue is relatively transparent. These effects have generated interest in the use of noble, alkali, and or other suitable metal nanoparticles as microscopic imaging labels. However, the large nanoparticle size relative to fluorophores and quantum dots may limit intracellular imaging applications. Resonant scattering applications may therefore more likely focus on particle tracking experiments and molecular contrast in tissues. For example, the two-photon luminescence from near infrared (NIR) resonant gold nanorods has been used to monitor microscopic blood flow in vivo, are featured in one invention embodiment. Single particle imaging has also been exploited in a release assay to sense the functional activity of a bio-molecular target, as opposed to simply measuring its concentration, and is another embodiment of the current invention.

As noted, the plasmon resonances of metal nanoparticles are sensitive to the dielectric properties of their local environment. This effect has also been exploited for a range of sensing strategies in which the presence of the molecule to be detected alters the extinction spectrum. In fact, one of the earliest demonstrations in the art of nanoparticle biosensing relied on this effect. Gold nanoparticles were functionalized with two-probe oligonucleotide sequences and exposed to a target oligonucleotide sequence, whose two ends were complementary to the probe sequences. When mixed, the target and probes hybridized, causing aggregation of the gold nanoparticles. The unhybridized gold nanoparticle solutions were red in color, typical of gold nanoparticles whose plasmon resonant peak absorption is 520 nm.

Upon aggregation, the plasmon resonances became severely damped, resulting in a blue color and eventually precipitation of the colloid. This dramatic change in spectral properties can be detected colorimetrically, yielding an extremely simple means of sensing specific oligonucleotide sequences, an application of considerable biomedical interest, and which is featured in one embodiment of the current invention. Other embodiments utilizing the technique feature an ability to detect single oligonucleotide base pair mismatches, sequence amplified DNA and reduce the limits of detection drastically. These are the result of chemical properties of the gold nanoparticles, such as their high density of DNA coverage and their ability to reduce silver to enhance the signals.

Analyte-induced nanoparticle aggregation can also be applied to protein and small molecule sensing, a concept that is not new. However, several advances in nanoparticle synthesis and conjugation have brought new capabilities to aggregation assays with plasmon resonant nanoparticles. Heterobifunctional cross-linkers used in one invention embodiment provide nanoparticle bioconjugates that are more stable under physiological conditions and are more resistant to fouling. For example, a poly(ethylene) glycol linker has been developed in the prior art with aldehyde distal to the nanoparticle surface. The aldehyde was functionalized with lactose to create a reversible and specific aggregation sensor sensitive to lectins. The advent of plasmon resonant nanoparticles tunable to the NIR (near infrared) has enabled detection in optically turbid media and is a feature in one embodiment. In the prior art, by using gold nanoshells immunoglobulins were detected in whole blood below ng/ml concentrations. Recently, aptamers have also been applied as the selective binding element rather than antibodies. With gold nanoparticles conjugated to aptamers for platelet-derived growth factor (PDGF), different aptamer-binding strengths to different PDGF isoforms could be distinguished and a competitive assay to detect PDGF receptors was demonstrated, and are a featured capability in one embodiment

As an alternative to the drastic optical changes due to nanoparticle aggregation, in one embodiment one can monitor the more subtle effect of the binding of a target molecule to the nanoparticle surface. In one embodiment, the presence of the target molecule alters the local dielectric environment of the nanoparticle, which shifts the localized surface plasmon resonance (LSPR) peak wavelength. Since inter-particle interactions are not required, the nanoparticles can be supported on a solid substrate to avoid aggregation and, therefore, improve stability of the sensor. When the current invention's bio-nanoparticles are coupled to antibodies or aptamers for specificity, LSPR sensors represent a very simple and potentially inexpensive strategy for sensing low-level, label-free analytes in complex media. LSPR sensing with nanoscopic arrays of silver triangles created by nanosphere lithography has proven highly effective. With such substrates, amyloid-β-derived diffusible ligands (ADDLs), a biomarker for Alzheimer's disease, have been detected at 10-pM concentrations in the prior art. Biomedical applications in one or more embodiments include LSPR and SPR sensing techniques.

LSPR sensing properties of several noble, alkali, and or other suitable metal nanostructures have been evaluated in the prior art by analyzing their spectra in media with different indices of refraction. The sensitivity is reported as ‘nm/RIU’ or ‘eV/RIU’, meaning the shift in LSPR peak wavelength or photon energy per unit change of refractive index. The range of observed sensitivity values demonstrates a strong dependence on the nanoparticle shape and composition. It has been highlighted recently that the plasmon resonant line width should also be considered in measurements of sensitivity, since the line width will affect the ultimate detectivity of an LSPR sensor. A unitless figure of merit (FOM) was therefore calculated, in which the sensitivity values described were divided by the resonance line width. According to this FOM, silver nanocubes, silver nano-triangles and gold nano-stars are the most highly sensitive nanoparticles. Although some core-shell nanoparticles in the art have shown the highest nm/RIU shifts, their FOM is low owing to the broad, low energy resonances exhibited by these nanoparticles. However, since grating-based instruments disperse light linear with wavelength and since some applications may be limited by spectral resolution rather than the signal-to-noise ratio, the nm/RIU shift could be the most significant parameter for certain sensing applications. The bio-nanoparticle configuration used in various embodiments for LSPR sensing will thus be strongly application dependent.

Drug delivery has also been accomplished in the prior art by associating the drug directly with a bioengineered clathrin bio-nanoparticle. Also in the art, DNA has been bound to lipid-stabilized gold nanorods. Upon resonant illumination, the nanorods transformed into spheres and the DNA was released without significant structural degradation, based on gel electrophoresis.

As in some other drug delivery art, liposome-supported plasmon resonant gold nanoshells (Troutman et al., Adv. Mater. 2008, 20, 2604-2608) can be used in the current invention. The plasmon resonant gold-coated liposomes are degradable into components of a size compatible with renal clearance, potentially enabling their use as multifunctional agents in applications in nanomedicine, including imaging, diagnostics, therapy, and drug delivery. This particular research demonstrated that laser illumination at the wavelength matching the plasmon resonance band of a noble, alkali, and or other suitable metal-coated liposome could lead to the rapid release of encapsulated substances, which can include therapeutic and diagnostic agents and is a feature in one embodiment. Leakage of encapsulated contents is monitored through the release of self-quenched fluorescein, which provides an increase in fluorescence emission upon release. Moreover, the resonant peak of these gold-coated liposomes is spectrally tunable in the near infrared range by varying the concentration of noble, alkali, and or other suitable metal deposited on the surface of liposomes. Varying the plasmon resonant wavelengths of gold-coated liposomes can provide a method in the current invention for spectrally-coding their light-mediated content release, so that the release event is initiated by the specific wavelength of light used to illuminate the liposomes. Spectrally coded release in controlled delivery of multiple agents to support complex diagnostic tests and therapeutic interventions is featured in another invention embodiment.

In another example embodiment, localized heating due to resonant absorption, also tunable into the near infrared, enables thermal ablation therapies, like using plasmon resonance absorption to kill cancerous tissues, and also for drug delivery mechanisms. In a manner similar to photodynamic therapy, in one embodiment plasmon resonant nanoparticles can be delivered systemically and activated locally by exposure to resonant illumination. The local temperature increase can also, in one embodiment, deliver drugs bound with the bio-nanoparticle or have a direct photothermal or thermolytic therapeutic effect. For example, in the prior art gold nanoshell-hydrogel composite materials have been loaded with protein and then illuminated at the plasmon resonance to stimulate release by shrinking the hydrogel. The nanoshells caused enhanced drug release and enabled multiple bursts of protein by modulated heating. In one device embodiment new types of non-immunogenic drug delivery applications are made possible, due to the well tolerated nature of the current invention's bio-nanoparticle. In one embodiment, stimulant energy is administered exogenously to the NIR resonant nanoparticles. A similar strategy has been pursued based on hollow polymer capsules filled with the substances to be delivered. In one embodiment, one or more types of encapsulating elements are impregnated with noble, alkali, and or other suitable metal nanoparticles so that absorption of light damages the walls of the encapsulating elements, thus releasing their contents, which method also features a unique capability to non-invasively pass an intact blood brain barrier and deliver drugs into the CNS.

In some types of therapeutic applications for treating cancer, thermal ablation treatments rely on the local application of heat to destroy diseased tissue selectively. Thermal therapies are simple and minimally invasive relative to conventional surgical treatments, although their effectiveness is limited by the ability to locally and specifically apply heat so as not to destroy healthy tissue. In one embodiment, plasmon resonant nanoparticles may also comprise highly effective enhancers of thermal ablation therapies since they can heat tissue locally owing to resonant absorption of energy and can be targeted to tumors. In one embodiment a cancer treatment employs plasmonic effects to destroy tumors.

In one embodiment, bio-nanoparticles with an outer layer of gold or other metal are administered into the body via one or more rotes of administration, including nasal delivery. The bio-nanoparticles embed themselves in a fast-growing tumor. When near-infrared laser light is applied to the area, it travels through the skin and induces resonant electron oscillations in the bio-nanoparticles, heating and killing tumor cells without harming the surrounding healthy tissue.

In another embodiment, noble, alkali, and or other suitable metal bio-nanoparticles photothermally destroy breast carcinoma cells in vitro with a continuous wave (CW) NIR laser energy dose that does not harm cells in the absence of the nanoparticles. Another embodiment approach uses bio-nanoparticles conjugated to gold nanorods.

In another embodiment, excitation with pulsed lasers further enhances the degree to which the bio-nanoparticles locally heat the target tissue since less time is available for the heat to diffuse from the nanoparticle. In one example in the prior art, gold nanoparticles of 30 nm in diameter have been conjugated to CD8+T lymphocytes and exposed to 20 ns, 565 nm pulses in vitro, resulting in selective loss of viability in the targeted cell population. Pulsed excitation has also been explored to selectively purge leukemic cells from a cell suspension. Gold nanoparticles of 30 nm in diameter were targeted to the leukemic cells and made to form clusters through the use of primary and secondary monoclonal antibodies. Upon pulsed laser excitation, these clusters form microbubbles owing to rapid heating at their surface, locally destroying the target cells thermolytically. These experiments demonstrate that pulsed laser excitation can localize thermal ablation therapies to the cellular level.

New types of lasers, called spasers, are also made possible with plasmonics and are featured in one invention embodiment. SPASER is an acronym for “Surface Plasmon Amplification by Stimulated Emission of Radiation”. In one spaser invention embodiment an element forms a counterpart of a laser, but it does not emit photons. It is analogous to the conventional laser, but in a spaser, photons are replaced by surface plasmons and the resonant cavity is replaced by a nanoparticle, which supports the plasmonic modes. Similarly to a laser, the energy source for a spaser bio-nanolaser embodiment is an active (gain) medium that is excited externally. This excitation field may be optical and unrelated to the bio-nano-spaser's operating frequency; for instance, a bio-nano-spaser can operate in the near infrared but the excitation of the gain medium can be achieved using an ultraviolet pulse.

The reason that surface plasmons in a spaser can work analogously to photons in a laser is that their relevant physical properties are the same. First, surface plasmons are bosons: they are vector excitations and have spin, just as photons do. Second, surface plasmons are electrically neutral excitations. And third, surface plasmons are the most collective material oscillations known in nature, which implies they are the most harmonic (that is, they interact very weakly with one another). As such, surface plasmons can undergo stimulated emission, accumulating in a single mode in large numbers, which is the physical foundation of both the laser and the spaser.

The spaser is thus the equivalent of a laser but it amplifies plasmon resonances rather than photons, with plasmonic resonators instead of a resonant cavity. Here the high-quality factor needed for lasing and amplification is a feature of the collective “coherent” response of the whole array.

In ordinary lasers, an optical cavity or optical resonator is an arrangement of mirrors that forms a standing wave cavity resonator for light waves. Photons bounce between the mirrors through a gain medium that amplifies the light. Light confined in the cavity reflects multiple times producing standing waves for certain resonant frequencies. Optical cavities are a major component of lasers, surrounding the gain medium and providing feedback of the laser light. The standing wave patterns produced are called modes; longitudinal modes differ only in frequency while transverse modes differ for different frequencies and have different intensity patterns across the cross section of the beam. [Note that ring resonators and whispering galleries are example of optical resonators that do not form standing waves.]

But the size of a conventional laser is dictated by the wavelength of the light it uses, and the distance between the reflective surfaces can't be smaller than half the wavelength of the light—in the case of visible light, about 200 nanometers. But a spaser embodiment gets around the half the wavelength of the light limitation by using plasmons. In contrast to conventional lasers operating at wavelengths of suitable natural molecular transitions, a lasing spaser embodiment does not require an external resonator and its emission wavelength can be controlled by metamolecule design. A spaser embodiment is thus a quantum amplifier of surface-plasmon emission.

One or more spaser invention embodiments have a wide range of applications, including diagnostic and therapeutic medicine, nanoscale lithography, probing and microscopy, to ultrasensitive surface-enhanced Raman scattering, to name some.

Bergman and Stockman first described the spaser phenomenon in 2003. Generally, optical devices are difficult to miniaturize because photons can't be confined to areas much smaller than half their wavelength. But devices that interact with light in the form of surface plasmons can confine photons within very small spaces. Trapping and sustaining light in these radically tight quarters creates extreme conditions in which the interaction of light and matter is strongly altered.

The first spaser device was announced in August 2009, created by researchers from Purdue, Norfolk State and Cornell universities. Noginov, et al., demonstrated an ingenious solution that took the form of a circular particle just 44 nanometers across. The 44-nm-diameter nanoparticles were used with a gold core and dye-doped silica shell that completely overcome the loss of localized surface plasmons by gain and realize a spaser. And in accord with the notion that only surface plasmon resonances are capable of squeezing optical frequency oscillations into a nanoscopic cavity to enable a true nanolaser it was shown that outcoupling of surface plasmon oscillations to photonic modes at a wavelength of 531 nm makes this particular system one of the smallest nanolasers and capable of operating at visible wavelengths. Transmission and scanning electron microscopy measurements gave the diameter of the gold core and the thickness of the silica shell as 14 nm and 15 nm, respectively. This shell size corresponds almost exactly to one embodiment of the current invention. In October 2009, Zhang, et al. also showed a nano-device that exploited plasmons to produce laser light.

Several types of spaser systems are currently known in the art. Each of these spaser systems, however, has distinct disadvantages. For example, some require elaborate cooling systems and cannot operate at room temperature, while some recent prior art overcomes this cooling limitation. By deliberately engineering surface plasmon activity occurring in a metal-dielectric interface, one example embodiment can sustain the strongly confined light long enough so that its oscillations can stabilize into the coherent state that is a key characteristic of a laser and operate in vitro at room temperature or work in vivo.

The use of nanostructured high-permittivity materials in one or more invention embodiments offer the possibility of tailoring the electric and magnetic response in metamaterials consisting only of a dielectric, thus removing the issue of losses at the source. In one embodiment, one or more types of metamaterials comprised of one or more suitable elements can be also be used to tailor the optical output to specific conditions by changing the structural and dielectric parameters of the constituents. Metamaterials are artificial media structured on a size scale smaller than the wavelength of external stimuli. Whereas conventional materials derive their electromagnetic characteristics from the properties of atoms and molecules, metamaterials in one embodiment enable the design of customized “atoms” and thus access new functionalities. Thus, the ability to tune and switch the properties of materials, something very rarely offered by nature, can be achieved in metamaterial embodiments.

In another example embodiment using metamaterials, a nanoscale optical device embodiment combines metamaterials with laser spasers to create a versatile planar source of coherent light.

Another significant invention embodiment is its forming the basis of a novel computing technology. The extra processing speed promised by bio-nano-plasmonic devices could generate novel and powerful applications in areas like cryptography. There are limitations in the field in the prior art, which are overcome in the current invention. Plasmons are density waves of electrons, created when light hits the surface of a metal under precise circumstances. Because these density waves are generated at optical frequencies, i.e., very small and rapid waves, they can theoretically encode a lot of information, more than what's possible for conventional electronics.

Plasmonics is thought to embody the strongest points of both optical and electronic data transfer. Optical data transfer, as in fiber optics, allows high bandwidth, but requires bulky “wires,” or tubes with reflective interiors. Electronic data transfer operates at frequencies inferior to fiber optics, but only requires tiny wires. Plasmonics, sometimes called “light on a wire,” would allow the transmission of data at optical frequencies along the surface of a tiny metal wire, despite the fact that the data travels in the form of electron density distributions rather than photons.

The main limitation to plasmonics today in computing is that plasmons tend to dissipate after only a few millimeters, making them too short-lived to serve as a basis for computer chips, which are a few centimeters across. For sending data even longer distances, the technology would need even more improvement. The key is using a material with a low refractive index, ideally negative, such that the incoming electromagnetic radiation, including any energy aspects is reflected parallel to the surface of the material and transmitted along its length as far as possible. Because there exists no natural material with a negative refractive index, nanostructured materials or metamaterials must be used to fabricate effective plasmonic devices.

In one invention embodiment, the current bio-nanotechnology invention represents a key improvement in the information science art, because the current invention's plasmonic nano-devices when used for data processing and communication can be a) self-assembled from a variety of suitable materials like proteins, b) with varying refractive indices and c) with different plasmonic properties.

Another problem with semiconductors for information processing is that their delicate conduction capabilities are vulnerable to ionizing radiation. Such rays can send avalanches of electrons streaming through delicate electronic components. At best, this corrupts data and halts calculations. At worst, it dramatically overheats transistors, permanently disabling them.

In contrast, the extra electrons that ionizing radiation can produce and their effects are minimal compared to the vast quantity of free electrons from which plasmons are generated in a metal. A plasmonic bio-nanodevice embodiment is thus able to process and store information in the harshest radioactive environments.

In one embodiment, hybridized bio-nano-plasmonic devices and electronics may coexist to mutual advantage in a single device. As the transistors in chips become smaller, the wires that connect them over distances of just a few nanometers become a significant bottleneck for data. Currently, such wires limit the speed at which electrons can deliver information, and an obvious solution is to replace them with photonic connections. The problem with such connections to date has been converting electronic signals into photonic ones and back again with a speed and efficiency that makes it worthwhile. Plasmons, which owe their existence to the easy exchange of energy between light and electrons, could overcome these limitations, making a hybrid electrical-optical embodiment a significant improvement over the prior art.

With respect to the prior art, a major drawback of semiconductor-based plasmonic elements and or hybrid electrical-optical chip systems is they typically involve a “top down” assembly approach, and employ some form of lithography and replication. Top down approaches can be time consuming, expensive and wasteful of materials. However, as noted, the current invention's bio-nano-plasmonic devices are automatically assembled from the “bottom up” using self-assembly techniques comprising proteins.

In addition to the above noted limitations, there are also other limitations in the prior plasmon art that are overcome by one or more embodiments of the current invention, and which limitations are summarized as follows:

i) Silica shells require specialized techniques for insertion of the metal (e.g., noble or alkali) nanoparticles that enable SPP's. Deposition of a coating onto a gold surface is not straightforward, and requires a clear understanding of the double layer properties of the material being coated, since the deposition must of necessity destroy the existing stabilizing layer of ions, charges or molecules at the surface. Yet, simultaneously, coalescence must be minimized. Gold metal has very little affinity for silica because it does not have an oxide film on its surface, and because there are usually adsorbed carboxylic acids or other organic anions present on the surface to stabilize the particles against coagulation. These stabilizers also render the gold surface vitreophobic. All these issues must be satisfactorily addressed when inserting a metal inside a silica shell. This problem is overcome in the current invention as one or more noble, alkali, and or other suitable metals can be used in one or more methods, such as, but not limited to: metal elements carried inside a self-assembling shell; metals forming a coating on a self-assembling shell; and or by metal coating an incomplete or partial shell element like a protein monomer.

ii) In addition, as a non-native foreign material, for in vivo biomedical applications silica shells require special functionalization, e.g., PEGylation, to minimize antigenic effects and for crossing cell membranes, and the like. This problem is overcome in the current invention as PEGylation of one or more invention bio-nanoparticle elements have been shown to be feasible.

iii) Further, many approaches in the prior art teach the requirement for an external excitation source to pump the plasmonic element, which is typically at the macro scale, and thus negates the promise of a completely nanoscale plasmonic element that also includes optical pumping sources. With respect to plasmonic elements using nanometer scale pumping sources, such as LED's, quantum dots, and the like, these sources are currently not considered suitable for in vivo applications. These problems are overcome in the current invention by using, in one embodiment, one or more types of suitable nanoscale excitation sources such as nano-scale bio-luminescent sources, but not limited to, that are also safe for in vivo use. These nanoscale excitation sources can be functionalized onto one or more invention bio-nanoparticle elements that also transport the plasmonic elements and their associated one or more types of electromagnetic radiation, including any energy aspects.

iv) Another current plasmonic limitation is that a silica shell, liposome, micelle, and the like, by their inherent physical properties, typically comprise just one vesicle element containing a single plasmonic element, significantly limiting plasmonic configurations and functionalization flexibility. This problem is overcome in the current invention in one example embodiment that comprises one or more cage, vesicle, shell shaped, and or other cavity forming plasmonic elements, whose associated multidimensional electromagnetic radiation, including any energy aspects are transported by a single invention element.

v) In another example of invention flexibility, metals and or metal coated elements may utilize one or more non-cage forming elements. In one example embodiment that overcomes plasmonic configurability limitations, no shells, vesicles nor cages are required to support SPPs and emit electromagnetic radiation. Instead, in one embodiment, one or more appropriately configured, metal coated, layered, and or structured metal coated surfaces comprising one or more clathrin and or coatomer partial protein cage elements of one or more types, like monomer elements, and of one or more molecular weights are used.

vi) Also, the physical properties of plasmonic elements like semiconductor-based and silica shell elements, but not limited to, limit their applicability for in vivo therapeutic and diagnostic applications in humans and animals, as there are the issues to overcome of tissue rejection, toxicity, adverse reactions, renal clearance, and the like. This problem is overcome in the current invention, as it has been shown that various materials, including metal elements can be transported safely and in a non-immunogenic manner by the invention's bio-nanoparticle.

vii) Also, plasmonic elements that utilize liposomes and lipid nanoparticles for in vitro and in vivo applications face many well-documented shortcomings, such as low encapsulation efficiency, rapid leakage of water-soluble drugs in the presence of blood components, and poor storage stability. These problems are resolved as it has been shown that the various limitations of liposomes and lipid nanoparticles are overcome by the invention's bio-nanoparticle.

viii) With respect to silica shells and semiconductors, and in the context of in vivo biomedical applications, their native inability to be readily functionalized with ligands and other biological elements represents a significant limitation. This problem is overcome in the current invention, as it has been shown that functionalization of the current invention's bio-nanoparticle is readily feasible.

ix) With respect to silica shells, semiconductors, liposomes, and other nanoparticle fabrication approaches such as polymers, dendrimers, and the like, and in the context of in vivo biomedical applications, their inherent inability to readily cross various biological barriers such as cells, as well as cross the blood brain barrier is well known. This problem is overcome in the current invention, as it has been shown that the current invention's bio-nanoparticle safely and non-invasively passes various biological barriers.

x) Further, silica shells, semiconductors, liposomes, and other nanoparticles, and in the context of in vivo biomedical applications, are not multi-drug central nervous system (CNS) nano-carriers capable of crossing or bypassing the blood brain barrier (BBB), delivering drugs inside cells, and enhancing effects of neuroprotective and neurotrophic drugs. This problem is overcome in the current invention, as it has been shown that the current invention's bio-nanoparticle safely and non-invasively passes an intact blood brain barrier while also transporting large and or small molecule elements.

xi) Also, there are two critical limitations on the selection of a metal for sensor construction. The surface exposed to light must be pure metal. Oxides, sulfides and other films formed by atmospheric exposure interfere with surface plasmon resonance. The metal must also be compatible with the chemistries needed to perform assays. Specifically, the chemical attachment of antibodies or other binding molecules to the metal surface must not impair the resonance. This problem is overcome in the current invention, as it has been shown that functionalization of the current invention's bio-nanoparticle via the chemical attachment of antibodies or other binding molecules is readily feasible, and equally important, such attachment is done not to the metal element, but to the protein element surrounding the metal element, leaving the metal element functionally unimpaired.
xii) In the prior art, Vitaliano, et al, teach a nano-laser approach using bio-nanoparticles composed of protein molecules. But critically, Vitaliano does not teach a plasmonic method like that taught in the current invention, whereby metals can absorb light by creating plasmons, which are particle-like collective excitations of conduction electrons at a metallic surface, and which plasmonic elements can also emit electromagnetic radiation. Also significant, Vitaliano only teaches a Q switched laser, a method not applicable to teaching a plasmon-laser-spaser embodiment as in the current invention, in the conventional meaning, at least—i.e. during the photon lifetime in the cavity. In addition, Vitaliano teaches a complete cage element, wherein in the current invention also teaches using a less than a completely formed cage element. This is a significant improvement, as a complete cage element is approximately 50-100 nm in size, while in the current invention an elements can be as small as 15 nm or even less. This much smaller size yields many more useful types of application embodiments, especially in vivo where very small element size can be important.

Thus, there exists a need for an improved plasmonic element and its associated one or more types of electromagnetic radiation, including any energy aspects that avoids the shortcomings of conventional designs, and the shortcomings are overcome by the current invention

### SUMMARY OF THE INVENTION

Plasmons are collective oscillations of the free electrons in a metal or an ionized gas. Plasmons dominate the optical properties of metal nanoparticles, which enables a variety of applications involving electromagnetic radiation and energy transport at nanoscale dimensions, single-molecule Raman spectroscopy, and photothermal cancer therapy, to name some. To be useful for surface plasmon resonance (SPR), a metal must have conduction band electrons capable of resonating with light at a suitable wavelength. A variety of metallic elements satisfy this condition. They include noble, alkali, or other suitable metals. Gold, a noble metal, is preferred in many, but not all SPR applications, as it produces a strong, easy to measure SPR signal in the near infrared region. It is also very resistant to oxidation and other atmospheric contaminants but it is sufficiently reactive to accommodate coating with a wide variety of binding molecules.

Plasmons also affect the spontaneous emission dynamics of optical emitters positioned in the vicinity of metal nanoparticles. The luminescence intensity can either be enhanced or quenched, depending on the geometry. Since the associated enhancements can potentially be several orders of magnitude, plasmon-enhanced luminescence is the subject of intense interest.

The invention, in one aspect, remedies the deficiencies of the prior art by providing a self-assembling, nanoscale, bio-nano-plasmonic element, which also may be employed in a scalable, bio-nano-plasmonic platform, which executes one or more functions and or effect one or more ends in vivo and or in vitro. A platform according to the invention may be used, for example, in biomedical, cosmetic, wellness, electronics, telecommunications, consumer, industrial, and information processing applications.

In one embodiment, at least one of the elements is or includes a plasmonic element that is capable of expressing one or more surface plasmon resonance states. In another embodiment, a plasmonic element is capable of expressing a plurality of surface plasmon resonance states. In another embodiment, a plasmonic element is capable of emitting electromagnetic radiation in one or more dimensions, configurations, and combinations.

In one embodiment, plasmons, although composed of many electrons, behave as if they were single charged particles, and part of their energy is expressed as an oscillation in the plane of the metal surface. In one embodiment, their movement, like the movement of any electrically charged particles, generates an electrical field.

In one embodiment, the plasmon nanoparticle's electrical field extends about 1 nanometer, to about 50 nanometers, and to even 100 nanometers or more above and below the metal surface, often in a perpendicular direction, but not necessarily.

In one embodiment, the interaction between the plasmon's electrical field and the matter within the field determines the resonance wavelength. In one embodiment, one or more changes in the composition of the matter within the range of the plasmon's field causes a change in the wavelength of light that resonates with the plasmon. In one embodiment, the magnitude of the change in the resonance wavelength, the SPR shift, is directly and linearly proportional to the change in composition.

As a general aspect, an element composed of one or more plasmonic elements and or non-plasmonic elements may take any suitable form, and multiple element embodiments may be further combined in any suitable manner to create multifunction, scalable platforms capable of expressing electromagnetic radiation in one or more dimensions, configurations and combinations.

In one or more embodiments, one or more types of metamaterials can be also be used to tailor an invention element's output to specific conditions by changing the structural and dielectric parameters of the constituents.

According to one embodiment, a self-assembling element is a functional substitute for one or more other types of plasmonic elements known in the art. In one embodiment, the current invention enables an analogous, biologically based, plasmonic element for one or more other types of plasmonic elements known in the art.

In one embodiment, an invention element is a source of plasmons, whose existence derives from the easy exchange of energy between light and electrons.

In one embodiment, an invention element comprises an electromagnetic energy emitting element, consisting of light, in one or more forms, of one or more different wavelengths.

In one embodiment, an invention element comprises one or more types of thermal energy elements, e.g., photothermal.

In one embodiment, an invention element comprises a quantum mechanical energy element.

In one embodiment, an element enables the controlled nature of resonance splitting, and comprises a simple optical scheme for plasmon excitation.

In general, in a further aspect, the invention is directed to a method of forming an element, including the steps of forming in vitro at least one element from self-assembling protein molecules, such as clathrin and or coatomer protein molecules forming one or more protein elements of one or more molecular weights, dimensions, geometries, symmetries, configurations and combinations.

In one embodiment, the invention comprises in whole and or in part one or more elements that emit surface plasmon enhanced electromagnetic radiation, including any other energies, of one or more types, composed of one or more types of elements having one or more inner layers and outer layers composed of one or more types of materials, including one or more layers that may further be comprised of metal surface elements of one or more types, and also may further comprise one or more elements formed and defined by structuring one or more metal layer surface elements, and also may further comprise one or more elements formed and or defined by placing one or more types of structure elements on one or more metal layer surface elements, wherein one or more layers are configured to completely surround, and or to cover at least some part of, one or more other layers, and at least one portion of one or more layers emits electromagnetic radiation of one or more types, including any energies, when one or more appropriate types of energies is applied to one or more layers.

In one or more embodiments, one or more elements of one or more dimensions, geometries, symmetries, configurations and combinations, and composed of one or more types of molecules, in whole or in part, comprise one or more plasmon-related structure elements having one or more surfaces, facets, properties, and or aspects.

In a preferred embodiment, at least one or more self-assembling Clathrin and or Coatomer I/II protein molecule elements of one or more types and or isoforms, including cloned isoforms, comprise in whole or in part one or more nanoscale elements with at least one or more plasmonic physics-related (“plasmonic”) properties, aspects, and or functions.

In a preferred embodiment, one or more self-assembling clathrin and or coatomer protein elements are formed in whole or in part from one or more purified, synthetic and or recombinant protein molecule elements and or their accessory elements.

In one or more embodiments, one or more protein-based elements and or suitable non-protein-based elements comprise in whole and or in part one or more types of enclosure elements that enclose completely and or partially enclose some portion of a cavity or a core element, thereby forming and or defining one or more elements such as a cage, vesicle, basket, sphere, shell, cup, bowl, and the like, of one or more dimensions, geometries, symmetries, configurations and combinations, and composed of one or more types of molecules.

In another embodiment, one or more enclosure-forming and or partial enclosure-forming elements carry one or more cargo elements of one or more types.

In another embodiment, one or more types of elements form one or more types of non-enclosure elements.

In another embodiment, one or more types of elements form a mixture of enclosure elements and non-enclosure elements.

In another embodiment, no elements of any type comprise any type of enclosure elements.

In another embodiment, one or more protein and or non-protein elements provide, enable, and or support one or more types of plasmon and or plasmonic-related energies and activities.

In one embodiment, the method includes internally or externally locating at least one plasmonic component element of one or more dimensions, geometries, symmetries, configurations and combinations, and composed of one or more types of molecules to one or more types of other elements, and which may also include one or more clathrin and or coatomer protein elements.

In one embodiment, the chemical attachment of antibodies or other binding molecules to the invention's protein element does not impair the surface plasmon resonance of the element supporting surface plasmon resonance, and represents an improvement in the art.

In one embodiment, a nanoscale element is larger than those described in the nanoscale plasmonic art, so the invention can incorporate a larger variety and number of plasmonic elements of one or more types, capable of expressing one or more types of electromagnetic radiation, including any energy aspects. In one embodiment, an element is directed towards providing an integrated, low-cost, reusable and sensitive plasmonic device.

In one embodiment, one or more plasmon dimer operations agree with the predictions of the classical approach for both plasmon energy and field enhancement. In another embodiment, quantum mechanical effects begin to significantly influence the plasmonic response of one or more dimers.

In one embodiment, an invention element comprises a nanoscale hybrid electrical-optical element that can be bioengineered using one or more methods known in the art, which makes the invention more versatile and cost-effective than the existing art.

In one embodiment, an element enables plasmon modes that retain the high sensitivity of plasmon resonances to the environment, but provide a local response and retains compatibility with one or more types of techniques, e.g., high throughput screening, required by biophysics.

In one embodiment, an element is an integrated bio-nanotechnology source of strongly confined surface plasmons in one or more types of waveguides, including, but not limited to, insulator dielectric, dyed dielectric, plasmon slot, metal-insulator-metal, other types of metallic slot waveguides, asymmetric shaped, ring shaped, and various other plasmonic waveguide configurations known in the art.

An element, in one embodiment, confines plasmons to a surface in a sandwich composed of a dielectric and another layer, and these surface plasmons propagate only in the thin plane at the interface. In one embodiment, a layer is composed of one or more types of molecules; for example, but not limited to, composed of metal molecules, for example, but not limited to a noble, alkali, and or other suitable metal abutting a dielectric. In one embodiment, these planar plasmonic structures act as wave-guides, shepherding the electromagnetic waves along a metal-dielectric boundary. In a preferred embodiment, surface plasmons are composed of electron oscillations that allow electromagnetic radiation, including any energy aspects to be localized, confined, and guided on sub-wavelength scales via an element.

In one embodiment, a plasmonics phenomenon takes place in the visible light and near infrared. In another embodiment, metal waveguide modes are in the microwave regime. In another embodiment, plasmonic modes become conventional waveguide modes as the frequency varies from visible light to microwaves. In one embodiment, given the metal permittivity at microwaves, the plasmonic dispersion becomes the conventional waveguide dispersion. In another embodiment, the surface plasmon dispersion at a single metal/insulator boundary can be extracted over a metal/insulator/metal (MIM) heterostructure if/when the metal-to-metal spacing is taken as infinite.

In another embodiment, a plasmonic elements and or non-plasmonic element may utilize exogenous and or an endogenous sources for surface plasmon excitation.

In order to excite surface plasmons in a resonant manner, an exogenous electron beam, or a coherent and or non-coherent light beam of one or more energies or wavelengths pumps an element in one or more embodiments.

An element, in another approach, may contain its own onboard, and or be sufficiently close to, a nanoscale optical pumping source composed of one or more types of molecules to excite the surface plasmons, making it a completely self-contained nano-device; which may, in one or more embodiments, function in vitro and or in vivo, and which makes the invention more versatile than the existing plasmonic art.

In one example embodiment, onboard, and or sufficiently close, quantum dots and or photonic dots (a collection of quantum dots) of one or more wavelengths and composed of one or more types of molecules comprise optical pumping sources for an element.

In another example embodiment, onboard, and or sufficiently close, light emitting diodes (LEDs) of one or more wavelengths and composed of one or more types of molecules comprise optical pumping sources for an element.

In one or more embodiments, functionalization of clathrin and or coatomer cage and or partial cage elements is done to provide optical pumping and or sensing for one or more plasmonic elements and or non-plasmonic elements when encountering one or more types of biological elements, e.g., bacteria, DNA, viruses, etc. In one example invention embodiment, but not limited to, an invention element is functionalized with bioluminescent proteins composed of more types of molecules (e.g., plant proteins, lux proteins, and the like), which may be used for optical pumping and or sensing.

An element, in another embodiment, provides greater configuration flexibility than that in the current art.

In one embodiment, one or more invention and or non-invention elements in one or more configurations and or combinations comprise one or more electromagnetic waveguide elements of one or more types that guide one or more waves, energies, or frequencies. In another embodiment, one or more waveguide elements may have one or more dimensions, geometries, symmetries, configurations and combinations, and be composed of one or more types of molecules. In another embodiment, one or more waveguide elements waveguide elements also may be comprised of one or more types of materials having one or more properties of one or more types.

In one embodiment, a vesicle comprises a size-adjustable element in order to adjust the dielectric gap, i.e., the plasmonic waveguide, at a nanoparticle-dielectric boundary. In one embodiment, the nanoparticle is a metal. In another embodiment, a waveguide/gap is dynamically adjustable during plasmonic operations. In one example embodiment, a waveguide/gap is about one nanometer in size. In another embodiment, a waveguide/gap is greater than one nanometer in size. In another embodiment, a waveguide/gap is substantially greater than one nanometer in size.

In one embodiment, one or more invention and or non-invention elements in one or more configurations and or combinations comprise one or more one or more dielectric elements of one or more types that are defined in one or more elements. In another embodiment, one or more dielectric elements define one or more gaps that support one or more desired wavelengths, frequencies, and or energy. In another embodiment, the desired dielectric loss and dielectric constant may also be observed and or controlled. In another embodiment, the desired properties of the dielectric element may not deteriorate in a given environment and or a desired lifetime.

In another embodiment, absorption losses are minimized by turning a plasmonic waveguide inside out by putting a dielectric at an element's core and surrounding it with a metal and or with a metal coated element composed of one or more types of molecules. In one embodiment, adjusting the thickness of a dielectric core changes the wavelength of plasmons.

In another embodiment, a dielectric contains one or more types of dye molecules known in the art that act as a gain medium.

In one example embodiment, one or more nanoparticle elements are spherical shaped elements. In another embodiment, one or more nanoparticles are dynamically adjustable in shape.

In another embodiment, one or more nanoparticle elements are nanorods composed of one or more types of materials. In another embodiment, upon resonant illumination, one or more nanorod elements are transformed into nanospheres. In one embodiment, the nanoparticles are regular, or symmetrical in shape.

In another embodiment, a nanoparticle element may be composed of a noncontiguous shell element featuring asymmetric geometry. In an example embodiment, a nanoparticle element is irregular, or asymmetrical in shape, and composed of a nano-core to allow for an excitation mixing of dipolar components in all plasmon modes of the particles. In one embodiment, such arrays enable highly tunable optical properties, and they render their optical properties dependent on the angle and polarization of the incident light. Besides their unconventional optical properties arising from the broken symmetry of a metal shell geometry, in another embodiment, these asymmetrical nanostructures possess a common feature of an enhanced localized electric field (“hot spot”) with a suitable excitation light compared to a full or non-broken shell nanostructure comprised of and or coated with, in whole or in part, one or more types of metal molecules.

In some cases, each of the elements is or includes a surface plasmon resonant element in one or more plasmonic states. Alternatively, some of the elements are or include non-surface plasmon resonant elements.

In one embodiment, an element provides electromagnetic and energy transport at nanoscale dimensions, in vivo and or in vitro.

In another embodiment, an element provides cargo transport of one or more types of molecular elements, like a drug cargo element, in vivo and or in vitro.

In another embodiment, an element enables single-molecule Raman spectroscopy.

In one embodiment, an element in one or more plasmonic states generates plasmons that affect the spontaneous emission dynamics of optical emitters positioned in the vicinity of one or more elements, in vivo and or in vitro. In one embodiment, the luminescence intensity can either be enhanced and or quenched, depending on geometry. In one embodiment, the associated enhancements of plasmon-enhanced luminescence can be several orders of magnitude.

In another embodiment, an element enables in vivo and or in vitro photothermal therapies, such as, for example, for treating cancer.

In another embodiment, an element enables in vivo and or in vitro photonic, photoelectric, and or other forms of electromagnetic or energy therapies like low-level laser light therapy for treating one or more types of medical, wellness, and or cosmetic conditions.

In one or more embodiments, an element, in whole and or in part, comprise ones or more laser and or spaser elements of one or more types that produce one or more collections of highly spatially and temporally coherent light of one or more wavelengths having one or more durations and or directionality. In another embodiment, so produced electromagnetic energy may be internally and or externally controllable and or tunable to one or more wavelengths, frequencies, and or energies of one or more forms or types.

In one embodiment, a plasmonic element forms a unitary bio-nanoparticle element with different parts that perform functions analogous to those in a conventional laser. In one embodiment, an element enables a laser spaser, whose surface plasmons work analogously to photons in a laser, in that their relevant physical properties are the same. In a normal laser, photons bounce between two mirrors through a gain medium that amplifies the light. But in one embodiment, the light in the form of plasmons bounces around on the surface of one or more metal nanoparticle elements, composed of one or more types of molecules, and which are encapsulated within an element. The energy does not dissipate rapidly from the metal nanoparticle's surface. Light from the spaser element can remain confined as plasmons or it can be made to leave the nanoparticle surface as photons in the visible-light range.

Prior art shows the non-linear optical properties of small aggregates of nanoparticles. These are extremely sensitive to shape and arrangement—very small changes can modify the emission, i.e. a far-field optical measurement can give information about a nanoscale deformation. In prior art, changing the shape of one or more metal (e.g., noble, alkali, and or other suitable metals.) nanoparticle elements allows one to modify the ratio of inter- to intra-band damping (Naomi Halas). In one embodiment, a plasmonic element's shape will have a role, but not only there. In one embodiment, deforming a nanocavity around a metal nanoparticle could also modify the emission pattern, i.e. lead to directional emission. In one embodiment, this would modify feedback and gain. In one embodiment, while it may not be possible to Q switch a spaser (in the conventional meaning, at least—i.e. during the photon lifetime in the cavity), it is instead about switching the gain/lasing on and off, and said switching is performed by one or more methods of externally stimulating and or inducing local deformation of one or more elements.

In one embodiment, one or more metal nanoparticles, e.g., noble and alkali metals, but not limited to, of one or more dimensions, geometries, symmetries, configurations and combinations, and composed of one or more types of molecules are encapsulated within an element, which may or may not also be filled with appropriate dye molecules known in the art, that act as a gain medium for one or more types of plasmonic elements.

In one embodiment, an element enables outcoupling of surface plasmon oscillations to photonic modes at one or more wavelengths. In other embodiments, one or more types of metamaterials are used to tailor an invention's output to specific conditions by changing the structural and dielectric parameters of the constituents.

In one embodiment, a plasmonic element of one or more types is pumped to supply the necessary energy. In one embodiment, pumping is accomplished by bombarding a nanoparticle element, which may be composed of a metal, with pulses of light of one or more energy or wavelengths.

A plasmonic element in one embodiment, may utilize an electron beam or a light beam of one or more energies that is exogenous to an element in order to excite the element.

A plasmonic element, in another embodiment, contains its own onboard, and or is sufficiently close to, a nanoscale optical pumping source composed of one or more types of molecules to excite the surface plasmons, making it a completely self-contained nano-device; which may, in one or more embodiments, function in vitro and or in vivo, and which makes the invention more versatile than the existing plasmonic art.

In one example embodiment, onboard, and or sufficiently close, quantum dots and or photonic dots (a collection of quantum dots) of one or more wavelengths and composed of one or more types of molecules comprise optical pumping sources for a plasmonic element.

In another example embodiment, onboard, and or sufficiently close, light emitting diodes (LEDs) of one or more wavelengths and composed of one or more types of molecules comprise optical pumping sources for a plasmonic element.

In one or more embodiments, functionalization of an element is done to provide optical pumping for a plasmonic element. In one example invention embodiment, but not limited to, an element is functionalized with bioluminescent proteins composed of more types of molecules (e.g., plant proteins, lux proteins, and the like), which may be used for optical pumping.

In another embodiment, optical pumping and or sensing for or more types of plasmonic elements is provided by one or more types of luminescence and or ultrabright fluorescence, including bioluminescence emanating from one or more other elements. Luminescent light can be directional and intense, but it is not coherent laser light. In one embodiment, fluorescent nanoparticles composed of one or more types of molecules work by absorbing light of one wavelength, then emitting it at another. In one embodiment, one or more optically transparent elements contain fluorescent molecule elements that are about one nanometer in diameter, while others may exceed 20 or even about 50 nanometers in diameter. In one embodiment, dye leakage from open channels is solved by incorporation of hydrophobic groups in liposomes, micelles, or mesoporous silica nanoparticles. In one embodiment, this approach keeps the synthetic approach compatible with virtually any dye molecule that can withstand the synthesis conditions, and yields ultra bright fluorescent nanoparticle elements.

In another embodiment, a plasmonic element may utilize an exogenous and or an endogenous excitation source.

In one or more embodiments, one or more invention elements may induce one or more quantum mechanical, plasmonic, and or lasing states and effects, and one or more elements execute one or more functions and or effect one or more ends in vivo and or in vitro.

In one or more embodiments, the invention is an improvement over other plasmonic art, because the invention enables, among other unique features:
1. Simplified nanoscale fabrication
2. Simplified plasmonic element and other element type attachment.
3. Cell and organelle crossing, and or membrane fusion.
4. Low antigenic, “green” nanotechnology.
5. Interaction, control, and regulation of cellular processes, like endocytosis, exocytosis, mitosis, trafficking and signaling, communication between cells, receptor upregulation and downregulation, other cellular behaviors, and the like.
6. Entering the CNS, including passing the blood brain barrier, and in some cases, in less than 30 minutes post administration.
7. One or more BNS elements that carry no additional elements like drug cargo, and can, operating alone produce an efficacious effect, thus acting like a drug, for example.
8. Hybrid invention elements composed of one or more types of non-invention elements, e.g., natural cell elements.
9. Self-modifying, orchestrated actions using natural control laws that govern biological elements.
10. Methods and behaviors defined by algorithms, in vivo and or in vitro.

In one illustrative embodiment, one or more elements can be of any suitable size. According to an illustrative embodiment, one or more elements are nanoscale elements.

According to one approach, various self-assembling and self-directed methods are employed. Unlike some plasmonic art where the plasmonic component elements must be fabricated into a structure; e.g., a silica shell, a semiconductor device, etc.; the invention, in one embodiment, provides individual protein molecules that self-assemble to form one or more plasmonic component elements, which makes the fabrication, integration, and addition of plasmonic elements easier to do. Nanoscale elements and or their platforms can be formed from the bottom-up, one element at a time. Another advantage of bottom-up fabrication is that it reduces the amount of superfluous material that surrounds each element, reducing the element's exposure to contaminant background radiation and thereby improving the functional effectiveness of the element.

In one embodiment, the invention features an element that includes a complete protein cage and or partial cage element like a protein monomer that enables one or more types of capabilities, including both plasmonic elements and non-plasmonic capabilities.

In one embodiment, one or more plasmonic component cargo elements of one or more types formed from one or more types of molecules are located within and or on a complete cage and or partial cage element. In another configuration, complete and or partial cage elements carrying one or more cargo elements may also include non-plasmonic elements. In another configuration, complete cage and or partial cage carrying elements cargo elements are exclusively non-plasmonic elements.

In one embodiment, a rigid, complete clathrin protein cage stabilizes its cargo. In another embodiment, a complete cage protein element environmentally sequesters its internal cargo and its contents.

In one embodiment, a complete clathrin cage lattice is stable, durable and about 100-fold stiffer than the typical liposome.

In an in vivo embodiment, a clathrin cage element is also resistant to pH changes and trypsin digestion.

In addition, the invention can maintain its structural integrity at room temperature in vitro and vivo, which eliminates the need for elaborate structure stabilizing mechanisms, like cooling systems. In one or more embodiments, an element operates at various temperatures, including at room temperature.

In one embodiment, an element comprises a relatively simple design that operates at the nanoscale, in vitro and or in vivo.

In another embodiment, an element comprises a medium to highly complex design, which may feature significant complexity and operates at the nanoscale, which makes the invention more versatile and cost-effective than the existing art.

In various embodiments, one or more elements may be of more than one functionalization type, and or express more than one type of functionality.

In various embodiments of the invention, an invention element is substantially larger than one nanometer in diameter, including sizes that can exceed about 50 or even about 100 nanometers in diameter.

In one embodiment comprising a complete cage, its relatively large size as compared to the existing art allows for a wider variety of possible plasmonic component cargo elements composed of one or more types of molecules, and which enables this invention embodiment to be highly flexible, affording one or a plurality of configurations.

In another embodiment, one or more cargo elements are functionally attached to the exterior of a complete cage and or partial cage element. In another embodiment, one or more cargo elements are both functionally attached to the exterior of a complete cage protein element and also located within the interior of a cage. These various types of cargo carrying capabilities makes the invention more versatile and cost-effective than the existing art.

In one embodiment of the invention, a partial cage element such as a partial clathrin monomer is larger than one nanometer, while a complete cage embodies sizes that can exceed about 45 nm in diameter.

In one embodiment, one or more types of complete and or partial cage elements cage element may be composed of one or more elements derived in part from one or more types of elements, for example, but not limited to, an amino acid sequence derived from a clathrin or coatomer protein. In one embodiment, clathrin complete and or partial cage elements are formed from purified, synthetic and or recombinant amino acid molecule elements and their residue elements, comprising in whole and or in part one or more types and or isoforms, including cloned isoforms.

In one embodiment, a partial clathrin cage element is composed of one or more 3-legged triskelia, each triskelion having 6 protein subunits; 3 clathrin heavy and 3 light chain subunits. In another example embodiment, the instant invention teaches one or more configurations as being composed of only 3 clathrin heavy subunits, forming a clathirn triskelia.

In another example embodiment, one or more complete and or partial cage elements are composed of Coatomer (COPI and COPII) proteins. Coatomer proteins are used instead of clathrin proteins, preferably in those applications where Coatomer characteristics would be more desirable than those of clathrin. In one embodiment, coatomer complete and or partial cage elements are formed from purified, synthetic and or recombinant amino acid molecule elements and their residue elements, comprising in whole and or in part one or more types and or isoforms, including cloned isoforms.

In one or more embodiments, one or more Coatomer I/II protein elements may be composed of one or more alpha, beta, beta′, gamma, delta, epsilon and or zeta subunits. Different combinations of these subunits are known to exist within Coatomer complexes. According to an illustrative embodiment, a Coatomer subunit is a nanoscale element.

Components of both COP1 and clathrin-adaptor coats share the same structure and the same motif-based cargo recognition and accessory factor recruitment mechanisms, which leads to insights on conserved aspects of coat recruitment, polymerization and membrane deformation. These themes point to the way in which evolutionarily conserved features underpin these diverse cell pathways.

In one invention embodiment, clathrin and coatomer elements and one or more methods may be used together in one or more configurations, taking advantage of their respective capabilities.

In one embodiment, the invention features an element that is less than a complete protein cage element, a partial cage element, which enables one or more types of capabilities, including both plasmonic elements and non-plasmonic capabilities.

In one embodiment, one or more complete and or partial cage elements, e.g., one or more clathrin triskelia and or coatomer subunits, are used to carry one or more types of cargo, in vivo and or in vitro.

In one embodiment, one or more complete protein and or partial cage elements carry one or more metal cargo elements composed of one or more types of molecules, and which cargo elements are capable in one or more embodiments of expressing one or more plasmonic states and or characteristics when the cargo element's surface plasmons are excited in a resonant manner.

In one embodiment, one or more complete and or partial cage elements may additionally comprise a hybrid molecular element formed from one or more other types of molecules.

In one embodiment, one or more types of complete and or partial cage elements are at least partially coated or completely coated in a metal composed of one or more types of molecules, and which is capable in one or more embodiments of expressing one or more plasmonic states and or characteristics when its surface plasmons are excited in a resonant manner, such as when a cage protein element is pumped by an electron beam, and or by a coherent and or non-coherent light beam of one or more energies or wavelengths.

According to one feature, one or more types of complete and or partial cage elements may be composed of and or coated with one or more types of metamaterials, and enable the design of customized “atoms”, and thus access new functionalities.

One advantage of the invention is that it inhibits charge transfer between the cage element and its cargo and prevents cage element distortion. Another advantage of inhibiting charge transfer is that it reduces limitations on the make up of transported cargo elements.

A charge inserted into a protein results in a local rearrangement of neighboring charged groups to maximize favorable electrostatic interactions. This is known as the protein dielectric response. It's a measurement of the difference in electrical charge from the inside of a folded protein compared to the outside (exposed) part of that same folded protein. That number is calculated and stays the same for that particular protein. There is a connection between the dielectric response and the pH dependent behavior of the protein.

In one embodiment, a charge transfer inhibiting protein cage element acts as a dielectric, or insulative element. In one embodiment, inside of one or more complete complete and or partial cage elements are located one or more cargo elements composed of one or more types of molecules, and the cage element inhibits charge transfer between the cage and its cargo.

In one embodiment, inside one or more insulative, complete cage and or partial cage elements are located one or more metal cargo elements composed of one or more types of molecules, and which cargo elements are capable in one or more embodiments of expressing one or more plasmonic states and or characteristics when the cargo element's surface plasmons are excited in a resonant manner.

In one embodiment, altering the pH dependent behavior of a complete and or incomplete cage protein elements is used to modify one or more electromagnetic or energy characteristics and or properties of one or more metal cargo elements.

In one embodiment, one or more cargo elements comprise natural, isolated, synthetic, and or recombinant elements.

In one embodiment, one or more cargo carrying elements include in whole or in part one or more non-invention elements of one or more types.

In one embodiment, one or more cargo elements and or cargo carrying elements comprise hybrid elements of one or more types.

In one embodiment, one or more elements of one or more types do not carry cargo elements.

In some embodiments, bonding and or attachment methods of one or more types, e.g., covalent, non-covalent, and any other bond type that can be explained by quantum theory, are used to directly attach, in one or more arrangements, one or more elements to one or more other elements.

In one embodiment, one or more elements each may bond and or integrate, physically and or logically, with one or more other elements of one or more types, including invention and non-invention elements.

In one embodiment, nanoscale ensembles comprising one or more types of elements allow for a large variety and number of possible cargo element configurations.

According to one feature, one or more functionalizing cargo elements can be, for example, mechanical, chemical, biochemical, biological, metabolic, covalent, non-covalent, ionic, photonic, sonic, acoustical, thermal, fluidic, electromagnetic, magnetic, radioactive, and or electrical elements, composed of one or more types of molecules. In one embodiment, such functionalizing elements may enable one or more types of plasmonic capabilities and or events. In another embodiment, such functionalizing elements may enable one or more types of non-plasmonic capabilities and or events.

In some configurations, an element carries cargo elements that include one or more metals of one or more types.

In other embodiments, and element-carried cargo elements are exclusively non-metal cargo elements that may include gases, as well as other cargo elements like drugs, optics, fluids, polymers, etc.

In other embodiments, the cargo elements are a mixture of metal and non-metal cargo elements.

According to one feature, the use of cargo and or coating elements composed of one or more types of metamaterials enable the design of customized “atoms” and thus access new functionalities.

In one embodiment, a cargo element, (e.g., shell, vesicle, cage, drug, but not limited to) is at least partially coated or completely coated in a metal composed of one or more types of molecules, and which is capable in one or more embodiments of expressing one or more plasmonic states and or characteristics when its surface plasmons are excited in a resonant manner, such as when a cargo element is pumped by an electron beam, and or by a coherent and or non-coherent light beam of one or more energies or wavelengths.

According to one illustrative configuration, one or more types of elements, such as cargo elements, may interfere with the invention's overall operation if carried in the same element as other element types. Instead, the problematic elements are carried in a separate element that exclusively carries non-interfering elements, thereby inhibiting disruptive interference of invention operations. Such non-interfering elements may be functionally and or physically linked with other elements carrying other element types.

In another embodiment, the invention takes full advantage of protein flexibility and plasticity to create plasmonic elements and or non-plasmonic elements of one or more types that are bonded, fastened, fused, and or affixed to one or more other elements, of one or more types.

In another embodiment one or more elements are functionalized, modified and or bioengineered using commercially available biotechnology tools and other tools and techniques known in the art, which makes the invention more versatile and cost-effective than the existing art.

A further advantage of the bottom-up self-assembly of an element is that it may, where it is desirable, enable the precise, highly ordered placement of cargo elements, with minimal inter-element spacings on one or more elements and structures, thus avoiding a drawback to the use of some prior art approaches that use free floating elements within a structure.

An invention element, in one configuration, may include one or more types of attachment molecules composed of one or more types of molecules for capturing and ordering the placement of one or more cargo elements inside a complete or partially complete cage. In one example embodiment, attachment molecules may also comprise receptor molecules that are natural, isolated, synthetic and or recombinant.

An invention element, in another functionalized configuration, may include adapter molecules; natural, isolated, synthetic and or recombinant, that may also be disposed between receptor molecules and one or more elements to couple the receptor molecules to another element, like to a cargo element.

In another element configuration, molecular or chemical bonding is used to attach directly cargo elements to an element. In other element configurations, a short molecular tether is used to attach cargo elements to a cage element.

In other element configurations, receptors, molecular tethers, and direct bonding are used in combination to attach cargo elements to a cage element.

An invention element, in one functionalized configuration, features ligands, natural, isolated, synthetic and or recombinant, including drugs, formed from one or more types of molecules, are attached to one or more elements.

In one configuration, one or more elements, of one or more types, are attached to one or more types of amino acids on one or more elements.

In another configuration, biotin-avidin is used as a coupler of one or more elements, of one or more types, to one or more elements of one or more types.

In another configurations, PEGylation, a cross-linker, molecular bridge, molecular tether, and the like are used to attach one or more elements, of one or more types, to one or more elements of one or more types.

In one example, molecules of one or more types are attached to a short molecular tether to one or more elements via site directed substitution mutagenesis, followed by reaction of a unique amino acid group with a specific molecular label.

In another invention embodiment, site directed mutagenesis is used to incorporate one or more elements, of one or more types, into one or more other elements, of one or more types.

In one embodiment site-directed mutagenesis using one or more types of primer; including its reverse complement; are used to insert one or more DNA sequences of one or more types into one or more coding regions of one or more elements.

In another embodiment, cloning is done of one or more genes encoding one or more elements. In another embodiment, one or more amino acids and or their encoder gene are controlled, regulated, modified, and the like, by one or more methods known in the art to produce an efficacious effect, in vivo and or in vitro.

In another illustrative embodiment, one or more elements, in one or more configurations, are coated in whole or in part with one or more elements, of one or more types, such as chemical, biological and or metallic materials, and the like. The coating elements may be or include organic, inorganic, and or synthetic materials, or a combination thereof.

In another illustrative embodiment, one or more elements, in one or more configurations, comprise one or more organic, inorganic, and or synthetic material elements, of one or more types, in one or more forms and or phases, in whole or in part

In one embodiment, one or more elements are radiation shielded, radio frequency (RF) shielded, thermally shielded, chemically shielded, optically shielded, and the like, in whole or in part, and in one or more configurations.

In one or more configurations, a cargo element is composed of a vesicle element transported by a cage element

In one or more configurations, a cargo element is composed of a vesicle element located within a complete protein cage element.

In another embodiment, there is more than one vesicle cargo element transported by one or more types of clathrin and or coatomer protein complete and or partial cage elements.

In one embodiment, there is a plurality of vesicle elements transported by one or more types of clathrin and or coatomer protein complete and or partial cage elements.

In one configuration, a vesicle is stabilized and protectively sequestered from its immediate environment by a complete cage and or partial cage element, adding further robustness to the vesicle.

In one embodiment, one or more vesicles may be composed of one or more types of molecules.

In one embodiment, one or more vesicle elements form plasmonic component elements. In another configuration, vesicle elements may also form non-plasmonic elements. In another configuration, vesicle elements are exclusively non-plasmonic elements.

In one embodiment, one or more nanoparticles, which may be composed of one or more types of molecules, of one or more sizes are cargo elements encapsulated within a vesicle. In one embodiment, vesicle encapsulated cargo elements may be composed of metal elements. In another embodiment, vesicle encapsulated cargo elements are also composed of non-metal elements. In another embodiment, vesicle encapsulated cargo elements are exclusively composed of non-metal elements.

In another embodiment, one or more cargo elements of one or more types are carried both by cage and vesicle elements. In another embodiment, one or more cargo elements are exclusively carried by either a cage protein element or vesicle element.

In various embodiments of the invention, a vesicle element is about one nanometer in diameter, including sizes that can exceed about 40 or even about 100 nanometers in diameter.

In one embodiment, vesicle-encapsulated nanoparticle cargo elements are about one nanometer in diameter, including larger sizes that can exceed about 40 nanometers or even about 100 nanometers in diameter.

In one configuration, attachment molecules extend through a vesicle to capture and position one or more cargo elements within a vesicle.

In another configuration, cargo elements within a vesicle may not be affixed to attachment molecules, and the cargo may be free floating within the cavity of a non-permeable vesicle, for example, in an encapsulated fluid or gas.

In another embodiment, one or more vesicles are functionally attached to the external surface of a complete cage and or partial cage element.

In another embodiment, one or more vesicles are located on both the exterior and within the interior of a complete cage and or partial cage element.

In one embodiment, a vesicle is loaded with a dielectric element composed of one or more types of molecules, e.g., a dye, or a gas, in which are suspended nanoparticle cargo elements composed of one or more types of molecules. In one embodiment, such nanoparticle and loaded vesicles enable one or more types of plasmonic capabilities and or events. In another embodiment, such nanoparticle and loaded vesicles enable one or more types of non-plasmonic capabilities and or events.

In one embodiment, nanoparticle and loaded vesicle elements are contained within complete, self-assembled clathrin and or coatomer protein cages. In another embodiment, nanoparticle and loaded vesicle elements are carried on complete protein and or partial cage elements.

According to one feature, a vesicle may be composed of one or more types of metamaterials, and enable the design of customized “atoms”, and thus access new functionalities.

In one embodiment, a vesicle element is at least partially coated or completely coated in a metal composed of one or more types of molecules, and which is capable in one or more embodiments of expressing one or more plasmonic states and or characteristics when its surface plasmons are excited in a resonant manner, such as when a vesicle element is pumped by an electron beam, and or by a coherent and or non-coherent light beam of one or more energies or wavelengths.

Generally, vesicle composition is directed to being composed of one or more types of suitable materials. In various embodiments, one or more vesicles may be composed of appropriate plasmonic-enabling and or non-plasmonic enabling materials known in the art. According to one embodiment, a vesicle is protein-based. According to a feature of this embodiment, the protein-based vesicle inhibits charge transfer between the vesicle and its enclosed cargo elements. According to another example embodiment, a vesicle is silica based. In another example embodiment, a vesicle is micelle-based. According to another example embodiment, a vesicle is metal based. According to another example embodiment, a vesicle is polymeric-based.

In one embodiment, a vesicle may be in the form of one or more types of liposomes. A liposome is a vesicle made out of the same material as a cell membrane. Liposomes can be filled with various types of elements, including drugs, dyes, etc. Liposomes, usually, but not by definition, contain a core of aqueous solution; lipid spheres that contain no aqueous material are called micelles, however, reverse micelles (those creating fibers) can be made to encompass an aqueous environment. Basically, a micelle is an aggregate of surfactant molecules dispersed in a liquid colloid. In one or more embodiments, a vesicle may be composed of one or more types of liposomal alternative elements, for example, micelles, silica shells, and other suitable vesicle-forming materials for various plasmonic elements and non-plasmonic enabling implementations.

In other configurations, a cage element and or a vesicle element may be devoid of cargo.

In some configurations, an element contains a single cargo element, while in other configurations it contains multiple cargo elements. In some configurations, cargo elements may also include one or more non-vesicle elements as cargo.

In one or more configuration, vesicle, cage element, and or cargo are coated completely and or at least partially in one or more types of coatings composed of one or more types of molecules.

In one illustrative embodiment, vesicle, cage element, and or cargo are coated completely or at least partially metal coated.

In one or more configurations, vesicle, cage element, and or cargo are coated completely and or at least partially in reflective and or non-reflective coatings composed of one or more types of molecules.

In general, in another aspect, the invention features a scalable platform that includes one or more embodiments of one or more elements.

In one embodiment, a scalable platform may also include an encoder for programming a plasmonic element composed of at least a subset of plasmonic elements and their one or more types of electromagnetic radiation, including any energy aspects, and a decoder for reading information from plasmonic elements, composed of at least a subset of plasmonic processing elements.

In general, in another embodiment, an element and or a platform may be physically and/or functionally cooperative with one or more other suitable types or forms of materials, substances, components, devices, or systems, in vitro and/or in vivo.

In one embodiment, one or more elements utilize, induce, respond to, and or exhibit one or more effects, such as quantum mechanical, mechanical, photonic, acoustic, electrical, biochemical and chemical, and the like.

According to one feature, the cage element, transported cargo elements, and or a vesicle respond to certain external and or internal stimuli, which can be, for example, mechanical, chemical, biochemical, biological, metabolic, covalent, non-covalent, ionic, photonic, sonic, acoustical, thermal, fluidic, electromagnetic, magnetic, radioactive, or electrical in nature. An example of such a stimulus response is enabling a plasmonic element or event, or one or more types of electromagnetic radiation, including any energy aspects.

In one example embodiment, switching gain/lasing of a plasmonic element by local deformation is a feature the current invention exploits for various effects. E.g., in one intra-cellular delivery embodiment, conformational changes to one or more invention elements produced upon coming into contact with a target (e.g., a stretch of DNA) are used to switch gain/lasing. In one embodiment, said conformational switching is used in addition to optical pumping sources, producing either more sensitive detection and or greater optical gain.

In one embodiment, a cage element is sued that has native multi-cargo capacity, and simultaneously may carry different types of cargo. According to one feature, one or more transported cargo elements are assay, diagnostic, therapeutic and or remediation elements of one or more types that are capable of operating in humans, animals, and plants, which makes the invention more versatile and cost-effective than the existing art.

Biomedical elements may be, for example, nano-structured and/or may include chemical, biological and/or metallic materials. The biomedical elements may be or include organic or inorganic materials or a combination thereof.

According to another feature, one or more cargo elements may be or include nanoscale diagnostic devices, biosensors, and/or prostheses, in any plasmonic/non-plasmonic element combination. Some or all of the plasmonic elements and non-plasmonic cargo elements, in one example configuration, may operate under the control and influence of other elements, including plasmonic elements and or non-plasmonic elements, and altogether may comprise a scalable platform for plasmonic-based biomedicine.

In other embodiments, one or more types of cargo elements can include, for example, one or more pharmaceuticals, biologicals, radioactive agents, gadolinium elements, iron oxide nanoparticles, diagnostic systems, or other nano-devices for in vivo delivery of targeted therapy to combat diseases. For example, targeted and/or high precision dosing can be done for different drugs: vaccines, antibodies, enzymes, receptor agonists or antagonists (DA, NMDA, GABA, opiate etc.), neurotrophic factors, genes for gene therapy, or small interfering RNAs (siRNA). Other Clathrin cargo elements may contain, for example, intelligent nano-prostheses that supplement or enhance cell, tissue, or organ functioning, thereby providing them with augmented capabilities. Nanoparticles are targeted to these regions to improve motor and cognitive functions.

Another biomedical advantage of the invention is that the cage protein material does not exhibit extreme hydrophobicity. A further advantage of the invention is that it provides a protein structure that can be bio-engineered to prevent in vivo and or cargo uptake by organs, tissue, and bone, and are secreted quickly and easily. In the converse, another advantage is that the protein material and or its cargo can be bio-engineered for highly selective uptake by targeted cells, tissue, organs, bone, as well as other organic and inorganic matter.

In another embodiment, one or more elements are also composed of one or more non-invention elements, e.g., one or more invention elements are conjugated to natural and or non-natural elements, like cells, optical devices, sensors, assays, etc., but not limited to, forming one or more types of hybrid elements in vitro and or in vivo.

The invention, in one embodiment, teaches one or more elements that dynamically and interactively respond to changing in vivo and or in vitro environments; e.g., change of pH, temperature, biochemical, or biological conditions, and the like.

In another embodiment, cargo elements are also composed of non-biomedical elements. In another embodiment, cargo elements are exclusively composed of non-biomedical elements.

In one embodiment, one or more elements, in one or more configurations, utilize self-directing, self-adapting, self-assembling, self-repairing, self-regenerating, self-regulating, and or self-replicating methods.

In one embodiment, one or more elements, in one or more configurations, utilize goal directed methods.

The invention, in one embodiment, provides one or more elements that maintain structural and or functional integrity long enough to do useful work, in vivo and or in vitro.

According to one feature, one or more elements re-supply, repair, reassemble and or regenerate defective, destroyed and or inoperable elements of one or more types, including non-invention elements, in vivo and or in vitro.

In another embodiment, one or more types of elements may exhibit no or limited immunogenic, toxic, and or environmental impact effects, and depending on cargo and other element type also may require little or no functionalization.

According to another feature, one or more elements are protected from the external environment, and the invention is stable with respect to dissociation and any element toxicity is sequestered from the surrounding in vivo and or in vitro environment.

In one embodiment, one or more elements in whole or in part may require minimal or no functionalization to be efficacious elements, like a drug and the like, but not limited to.

In another embodiment, one or more elements in whole or in part comprise one or more structures, of one or more types.

In one bioengineered embodiment, invention elements comprise self-assembling scaffolds, which may also be functionalized in one or more ways via one or more methods known in the art.

In another aspect, a cage element features no cargo elements at all. According to one embodiment, empty, self-assembling complete and or partial cage elements include highly ordered scaffolding and a charge transfer limiting substrate material for self-assembling multi-layer, multi-plasmonic element systems.

In another embodiment, one or more complete and or partial cage elements may facilitate the self-aligning of cargo carrying elements with respect to one another.

In another embodiment, one or more elements in whole or in part comprise a shape programmable and or shaped scaffolding system via which one or more elements of one or more types form one or more structures with one or more types of shapes and or functions.

In another bioengineered embodiment, invention elements comprise self assembling monolayers (SAM) prepared using invention biopolymers deposited in an ordered manner and uniform thickness on elastomers, silicates, noble, alkali and other metals, or other metallic monolayers, and the like. In one embodiment, an optical detection systems uses invention-based SAMs. In one SAM embodiment, an optical detection system relies on surface plasmon resonance. The binding of one or more target elements to one or more such invention elements results in changes in the intensity of light emerging from the evanescent wave. In one embodiment, one or more invention-based SAMs are coated onto one or more type of surfaces, e.g., thin films, optical fibers, and the like.

In one or more embodiments, invention biopolymer elements exhibit one or more material properties of interest to one or more industrial sectors, such as electronics, optics, medical devices, and pharmaceuticals, but not limited to. Example applications of invention biomaterials and elements include, but are not limited to, the design and construction of nanoscale integrated circuits, of laminated structural elements, of nanosensors that can be embedded in persons, animals, and other organisms, or placed on other elements composed of one or more types of materials.

In one embodiment, one or more elements act as one or more types of efficacious replacements for one or more other elements, including non-invention elements, in vitro and or in vivo, e.g., act as replacements for one or more natural elements commonly found in cells, but not limited to.

In some configurations, one or more elements comprise a cargo element, while in other configurations they comprise multiple elements, of one or more types. In some configurations, one or more or each of the elements and or cargo elements is a metal, and or may include one or more metals. Alternatively, each of the elements and or cargo elements is or includes non-metal elements. In other embodiments, elements and or cargo elements are exclusively non-metal elements that may include gases, as well as other elements like biological elements, drugs, optics, polymers, etc. In another embodiment, one or more elements and or additional elements comprise one or more types of material forms, including a solid, gas, vapor, crystal, and the like. In another embodiment one or more invention and or non-invention elements, in one or more combinations, comprise one or more types of isolated, synthetic and or recombinant elements.

The invention, in one embodiment, provides for a plurality of elements comprising aggregated, complex self-assembled nanoscale structures that dynamically bind together one or more types of endogenous, exogenous, homogeneous, and or heterogeneous elements into one or more complex elements, which also may have one or more payload types.

The invention, in one embodiment, provides a capability for in vivo and in vitro integration of one or more types of elements into other elements, devices and mechanisms, some of which may also be non-invention elements, that also may be linked together functionally or logically, including with other devices and or operators, locally or at a distance, significantly enhancing the overall capabilities of the invention.

In one or more embodiments, an invention element enables electromagnetic radiation, energy, and or cargo transport at nanoscale dimensions, in vivo and or in vitro. In one embodiment, an element is a nanoscale electromagnetic element capable of inducing, emitting, and or generating one or more wavelengths, types, and or forms of energy. In one embodiment, energies are photonic and may be coherent and or non-coherent. In another embodiment, energies are photothermal. In another embodiment, electromagnetic energies are photoelectric. In another embodiment, energies are quantum mechanical. In one embodiment, one or more energies induce and or produce one or more effects on one or more types of non-invention elements.

In various embodiments, one or more invention elements may operate in vitro and or in vivo to form one or more types of quantum medicine elements. Quantum medicine is typically described as multidisciplinary research using quantum physics to show that the human body can be controlled and regulated by the human energy system. It is also described as a branch of medicine that can manipulate the body's own energy states to treat and prevent disease.

In practice, because all elements when sufficiently small, e.g., at the nanoscale, are governed and or subject to influence by natural quantum mechanical forces, it is feasible that these forces can be harnessed and deliberately used in healthcare for assay, diagnosis treatment and of various conditions. Quantum mechanical effects, for example, are routinely exploited in medical imaging, such as for magnetic resonance imaging (MRI).

In the brain, in one embodiment, the neuronal membrane represents a liquid crystalline medium whose anisotropic properties can support various types of quantum medicine applications. In one embodiment, inducing quantum coherence in ‘diseased’ neurons supports one or more types of quantum medicine therapies and or diagnostics.

In various invention embodiments, one or more types of energies may be used singly and or in combination. In an example embodiment, because currently used brain stimulation techniques cannot stimulate specific neurons implicated in brain diseases, one or more in vivo, targeted invention elements enable neurons to simultaneously sample all their potential quantum, optical, bio-energetic, and or electromagnetic pathways, then enable the most appropriate energy and beneficial pathway and thereby return the diseased neuron to a healthy state. In some embodiments, one or more neuronal biochemical energies are also stimulated and managed.

In one or more embodiments, the invention's emitted electromagnetic radiation, including any energy aspects relate to one or more types of elements and platforms, such as a biomedical platform, a quantum medicine platform, telecommunication platform, environmental remediation platform, computational platform, and the like, using such elements.

In one embodiment, an element enables a wide range of plasmonic applications in bio- and chemical sensors. In one embodiment, surface plasmon resonance is used as the basis for a sensor that is capable of sensitive and quantitative measurement of a broad spectrum of chemical and biological entities.

In one embodiment, an element photonically excites plasmons to induce reaction and detect interactions between DNA, proteins, and adenovirus, and whose applications include sensors, polymerase chain reaction, silicon optics, and the like.

In one embodiment, an element is a low-level light therapy element that operates in vivo and or in vitro, in a variety of environments and conditions, which are capable of operating in humans, animals, and plants, which makes the invention more versatile and cost-effective than the existing art.

In one embodiment, an element a therapeutic, diagnostic, prosthetic, sensor, and or assay element that operates in vivo and or in vitro, in a variety of environments and conditions, which is capable of operating in humans, animals, and plants, and which makes the invention more versatile and cost-effective than the existing art.

In one embodiment, an element alters the behaviors and or compositions of one or more types of cells and or other types of biological elements, which is capable of operating in humans, animals, and plants, and which makes the invention more versatile and cost-effective than the existing art.

In one embodiment, one or more elements are adjunctive therapy elements, whose purpose is to assist a primary treatment element, like a drug element, which may or may not also comprise a cargo element, in vivo and or in vitro, in a variety of environments and conditions. This is an embodiment that makes the invention more versatile and cost-effective than the existing art.

In another embodiment, one or more elements composed of isolated, synthetic, and or recombinant clathrin and or coatomer protein elements may further comprise one or more types of inherently efficacious protein elements, in vivo and or in vitro, and these protein elements may act as assistive, adjunctive therapy elements to one or more plasmonic element and its electromagnetic radiation elements, including any energies. This is an embodiment makes the invention more versatile and cost-effective than the existing art.

In another embodiment, one or more inherently efficacious protein elements, together with one or more elements act in concert as adjunctive elements, and whose concerted purpose is to assist a primary treatment element, like a drug element, which may or may not also comprise a cargo element, in vivo and or in vitro, in a variety of environments and conditions. This is another embodiment makes the invention more versatile and cost-effective than the existing art.

In one or more embodiments, in whole or in part, and in one or more in vivo and or in vitro applications and or processes, one or more current invention elements directly and or indirectly have, produce, and or participate in, one or more interactions of one or more types that result in one or more invention and or non-invention element properties to be altered, enhanced, or otherwise transformed, including such element properties as, but not limited to, structural, permeable, soluble, adjuvantive, compositional, efficacious, operational, physiological, biological, cellular, genetic, pharmacokinetic, pharmacodynamic, toxicokinetic, and the like, and or one or more such element interactions and resulting properties that yield one or more novel elements and or medicaments of one or more types.

In another embodiment, free radicals, toxic elements, other types of undesirable elements and the like circulating within an in vivo and or in vitro environment are scavenged via molecular tethers; via other elements of one or more types attached to one or more invention elements; and or via direct binding to one or more elements; which undesirable elements are then efficaciously acted upon by one or more elements.

In one embodiment, an element varies the plasmon resonant wavelengths of one or more invention cage, shell and or vesicle plasmonic elements, which also may be coated in whole or in part with one or more types of metal molecules. In another embodiment, an element provides a method for spectrally-coding light-mediated cargo content release from one or more vesicle elements and or complete and or partial cage elements, so that the release event is initiated by the specific wavelength of light used to illuminate the vesicles and or complete and or partial cage elements.

In one embodiment, spectrally coded release enables applications in controlled delivery of multiple agents to support complex diagnostic tests and therapeutic interventions. In another embodiment, the resonant peak of one or more vesicles and or complete and or partial cage elements is spectrally tunable in the near infrared range by varying the concentration of metal, e.g., a noble, alkali, and or other suitable metal, deposited on the surface of vesicles and or complete and or partial cage elements.

In another embodiment, an element provides a method for spectrally-coding light-mediated effects and or other forms of electromagnetic emission effects on one or more in vivo and or in vitro biological elements, like a cell, virus, bacteria element, and the like, for therapeutic effect.

In one embodiment, the well-known permeability increase at the phase transition temperature provides a means to trigger release of a transported agent element, like, for example release of a therapeutic agent in locally heated tissues. In one embodiment, plasmonic electromagnetic emissions and or coherent laser spaser emissions, sometimes along with local tissue heating cause one or more agents trapped in one or more thermally sensitive vesicle and or cargo elements to be triggered and released, thereby forming a targeted agent delivery system. Diagnostic and therapeutic agents may be simultaneously delivered via this site-specific delivery embodiment by using one or more thermally and or optically sensitive elements,

In the prior art, plasmonic elements are not readily endocytosed (enter) into a cell and access cell DNA. But in one embodiment, one or more invention elements are readily endocytosed, and therefore represent an improvement in the art. The current inventors have shown that bioengineered clathrin can easily do this. (Endocytosis is the primary job of clathrin in the body.) The current inventors also have shown that unmodified clathrin, unlike all other nanoparticles, can readily cross an intact blood brain barrier and deliver both large and small molecular payloads into the central nervous system. In one or more biomedical embodiments, this cell/membrane crossing feature is beneficially exploited and is a significant improvement over prior art.

In other embodiments, the difficulty and complexity of in vivo and in vitro application functionalization of some types of nano-plasmonic art; e.g., silica shells, liposomes, semiconductors, and the like; is overcome. Clathrin complete and or partial cage elements, and clathrin-coated vesicles have a native ability to simultaneously carry different types of cargo elements, such as: antibodies, lux genes, hormones, growth factors, and neurotransmitters.

In one embodiment, a complete, rigid clathrin protein cage stabilizes its cargo and or vesicle, and environmentally sequesters its contents. The clathrin lattice is also durable. A complete cage element is about 100-fold stiffer than the typical liposome, and both complete and or partial cage elements in general are resistant to pH changes and trypsin digestion.

A cage element also has multiple amino groups that can easily be modified (e.g., lysine, cysteamine). These manifold qualities make clathrin complete and or partial cage elements much more suitable for various in vitro and in vivo applications than that described in prior art.

Another unique feature of the current invention is that clathrin has inherent cell signaling properties, a feature absent in plasmonic nanoparticles in the art, including liposomes, dendrimers, pluronic micelles, silica shells, etc. In some naturally occurring instances, clathrin is also co-opted by bacteria to gain cellular entry. In one or more embodiments, the cell signaling properties of clathrin elements is exploited for efficacious effect, in vivo and or in vitro.

Some embodiments include a molecule having an unpaired electron, a transition metal ion, which can be found in the active centers of many proteins (metalloproteins), or a material having any defect that produces an unpaired electron.

According to one in vivo application for enhanced medical imaging, one or more invention elements are also functionalized with cargo elements comprising paramagnetic lanthanide, transition metal ion complexes, and the like, enabling new types of hybrid imaging systems that combine NMR with plasmonic imaging techniques; representing an improvement in the art.

In another illustrative embodiment, one or more elements also accept free radical molecules such as nitroxide molecule spin labels for plasmonic/electron paramagnetic resonance (EPR) based invention applications; representing an improvement in the art.

In another illustrative embodiment, one or more elements accept and or comprise in whole or in part one or more types of element label and assay strategies, and instruments for detection of one or more such labeled and or assay elements. In one or more embodiments, hybrid elements may include, but are not limited to: fluorescence and confocal microscopy elements, flow cytometry elements, laser scanning cytometry elements, fluorescence microplate analysis and biochip elements, immunoassays (for protein hormones, drugs, steroids, immunoglobulins, viruses, whole bacteria, and bacterial antigens), quantitation of halothane anesthetic gases, and DNA binding assays, nucleic acid-based diagnostic elements, and the like. In various embodiments, one or more hybrid elements meet and or surpass the requirements for label and assay sensitivity, accuracy and convenience; representing an improvement in the art.

In another illustrative embodiment, one or more elements accept and or comprise in whole or in part one or more types of high throughput applications, e.g, in an automated clinical chemistry analyzer. In one embodiment, this finds application in label free quantitative immunoassay techniques for proteins and small analytes, in conformational studies with proteins as well as real time association dissociation measurements of receptor ligand interactions for high throughput screening and lead optimization.

In one embodiment, applied SPR is used with colloidal metal particles, e.g., noble, alkali, and or other suitable metals, in one or more types of buffered solutions. In one embodiment, a colloidal application offers advantages over conventional SPR, as the support is inexpensive, easily synthesized, and can be coated with various proteins or protein ligand complexes by charge adsorption. For example, with a colloidal metal, the SPR phenomenon can be monitored in any UV spectrophotometer.

In another illustrative embodiment, one or more elements accept and or comprise in whole or in part one or more types of environmental monitoring elements.

In another illustrative embodiment, one or more elements accept and or comprise in whole or in part one or more types of agriculture pesticide and antibiotic monitoring elements.

In another illustrative embodiment, one or more elements accept and or comprise in whole or in part one or more types of food additive testing elements.

In another illustrative embodiment, one or more elements accept and or comprise in whole or in part one or more types of military and civilian airborne elements.

In another illustrative embodiment, one or more elements accept and or comprise in whole or in part one or more types of biological and chemical agent testing elements.

In another illustrative embodiment, one or more elements accept and or comprise in whole or in part one or more types of real time chemical and biological production process monitoring elements.

In one or more embodiments, one or more elements accept and or comprise in whole or in part one or more types of elements for the measurement in real time of the kinetics of ligands receptor interactions and to the screening of lead compounds in the pharmaceutical industry, but also to the measurement DNA hybridization, enzyme-substrate interactions, in polyclonal antibody characterization, epitope mapping, protein conformation studies and label free immunoassays.

In another embodiment, one or more types of cargo elements such as comprising in whole or in part one or more large molecule elements, small molecule elements, optical elements, cargo elements, agent elements, device elements, drug elements, and the like, enter the CNS, including passing the blood brain barrier, in 30 minutes or less and or in 30 minutes or more, post administration, and, depending on cargo and other element type, may require minimal functionalization for such cargo element passage.

In one embodiment, one or more elements of one or more types comprise targeted and or non-targeted drug elements, biological elements, other forms of healthcare elements, including cosmetic elements, in one or more configurations or combinations, for diagnosing, remedying, inhibiting, mitigating, curing, and or preventing one or more types of diseases, infections, physical or mental trauma, other forms of physical and mental afflictions, and the like, of one or more types, including types featuring minimal immunogenic and or toxic effects.

In one embodiment, one or more elements are used as a means for evaluating drug advancement and efficacy.

The invention teaches a biological model and or method that is consistent from a minimalist component level up, e.g., amino acid residues comprising in part one or more clathrin and or Coatomer I/II proteins of one or more isoforms, making drug discovery safer, more efficacious, more time and cost effective, and overall, a much more rapid process.

In one personalized medicine embodiment, the invention reduces drug side effect profiles and or produces greater agent efficacy, as well as excludes agents that may have no efficacy in a particular individual. The invention, in one embodiment, provides for individual patient factors such as genotype, phenotype, age, gender, ethnicity etc., to be taken into account by one or more elements and factored into dosing and administration consideration.

In one embodiment, one or more cargo elements comprise one or more types of pluripotent stem cells and or comprise one or more stem cell delivery methods.

In one or more embodiment, one or more current invention elements, in whole and or in part, via one or more methods execute one or more functions, effect, and or induce one or more ends of one or more types involving one or more cells, cellular pathways, cell processes, cell components, cell compositions, internal and or external cell processes, cell development activities, cell regeneration activities, genotypes, DNA elements, RNA elements, genetic expressions, epigenetic activities and behaviors, and or one or more phenotypes, and the like, of one or more types, for the treatment of one or more conditions associated with one or more types of illnesses and or traumas in humans, animals, plants, and fungi, and or for their general betterment.

According to one feature, one or more cargo elements may be or include one or more research, therapeutic, diagnostic, vaccine, assay, and or prosthetic agents, in one or more configurations, and thereby constitute one or more types of biomedical elements. Such biomedical elements may be, for example, nano-structured and/or include chemical, biological and/or metallic materials. The biomedical elements may be or include organic, inorganic, and or synthetic materials, or a combination thereof.

Medical, biomedical, bioengineered, and or biological applications and platforms of the instant invention may include, but are not limited to, imaging; sensor; genetic and protein assay; diagnostic; drugs and drug delivery; prosthetic; inter- and extra-cellular tissue; whole organ; circulatory system; medical device; implantable defibrillator; pacemaker; coronary stents; angioplasty device; and other like applications.

In one embodiment, one or more elements comprise one or more applications that perform analysis, of one or more types, of disorders of complex inheritance.

In one embodiment, one or more elements comprise one or more applications that perform analysis, of one or more types, of pharmacologic therapy.

In one embodiment, one or more elements comprise one or more types of prognosis and therapy selection—“theradiagnostics”.

In one embodiment, one or more elements comprise one or more genomic applications of one or more types.

In one embodiment, one or more elements comprise one or more oncology applications of one or more types.

In one or more embodiments, one or more elements may use routes of administration comprising one or methods of one or more types, such as those defined by CDER Data Element Number C-DRG-00301 in the US FDA Data Standards manual. Routes of in vitro administration of one or more elements may also comprise one or more forms.

In one or more embodiments, one or more pharmaceutical and drug formulations of one or more types are used, in whole or in part, by one or more elements, such as: tablets, capsules, soft galantine capsules, topical, injections, eye drops, syrups and liquids, soap and cosmetics, birth control devices, and the like, but not limited to, as well as one or more types of biologics, chemical compounds, water soluble compositions, and the like, but not limited to. In vitro formulations may also comprise one or more formulations of one or more types in one or more embodiments for consumer, commercial, and industrial applications.

According to one feature, one or more elements respond to one or more external and/or internal stimuli, which can be, for example, mechanical, chemical, biochemical, biological, metabolic, covalent, non-covalent, photonic, sonic, acoustical, thermal, fluidic, electromagnetic, magnetic, radioactive, quantum mechanical, or electrical in nature. Examples of such a stimulus response is altering a cargo element carried by an element; the altering of the element itself; causing changes in cellular process like endocytosis, exocytosis, mitosis, trafficking and signaling, and the like, including other conformational changes.

In another embodiment, photonic energy impacting one or more elements produces electrical current and/or photonic energy.

In general, in another embodiment, one or more elements and or platforms are physically and/or functionally cooperative with other suitable types or forms of elements, agents, organisms, materials, substances, components, devices, and or systems, including non-invention elements, in vitro and/or in vivo.

In one embodiment, the invention provides for the ability of one or more elements to sense, track, recognize, attack and or destroy multiple targets on the fly, in vivo and in vitro, using dynamic target prioritization for a single element type and or multiple element types.

In one application, one or more elements, including cargo elements, comprise one or more types of targeted agent delivery systems and or agents in vivo or in vitro, including high precision dosing, using, as appropriate, ligands, targeting moieties, and or other vectors. In one application, one or more targeted elements comprise one or more research, remedial, inhibitory, mitigation, preventive, prosthetic, assay, and or other type of consumer, commerical, and industrial bio-molecular agent or device, in one or more combinations, and may altogether comprise a unified element and or platform.

The invention, in one embodiment, provides for a method for targeted delivery systems that leverage and utilize biological control laws and that may act as self-directed systems.

According to another invention embodiment, one or more targeted elements may use molecular-imprint technology, which is used for the production of molecule-specific cavities that mimic the behavior of receptor binding sites, without the temperature sensitivity of natural systems.

According to another feature, biodegradable films may also be used as a pliable template for one or more targeted elements, which are pressed into a biodegradable film and then removed, leaving a physical mold of the element's shape. The film can then be hardened and used by an element to detect a particular element, which may be, but is not limited to, a particular receptor, protein, or cell, since its complex imprint shape on the film will bind only to that particular biological element.

In one embodiment, the invention provides for a targeting system using biodegradable nanocapsules for delivery of one or more elements in vivo or in vitro.

In another application, a nanoscale platform composed of a plurality of elements performs molecular-level and or cellular-level target site loitering, monitoring, repair, construction and or dynamic, interactive control and regulation of biological systems, in vitro and in vivo.

In another embodiment, one or more elements, including in whole or in part one or more non-invention elements, operating alone or with one or more additional elements, comprise one or more types of membrane fusion elements. In one embodiment, the resulting biological processes and interactions from such fusion may lead to a series of controlled, regulated, extended, modulated, purposefully, and or self-directed methods and or behaviors of elements.

In one example embodiment, one or more elements in whole or in part execute one or more types of actions involving conformational changes, bonding, attachment, and or the fusion of one or more elements to a cell membrane, one or more of which actions may lead to changes in cellular processes, such as endocytosis, exocytosis mitosis, trafficking and signaling, and the like, and or enable the precise dispatch and sequenced delivery of selected agents from an element to a target cell. Alternatively, a series of interlocking steps between a part of a cell membrane, and all, or a subset of the materials comprising an element may cause the cessation of one or more element's delivery to a target cell, and or enable delivery from other sources.

In another configuration, one or more elements dynamically respond to natural environmental conditions and manifest special functions. The various control laws that regulate biochemical reactions and physiological processes often display features that allow biomolecules or biological structures to perform more tasks than are reasonably expected from a simple mechanical device. In one embodiment, the invention takes deliberate advantage of these biological control laws. Via the use of bio- and genetic engineering methods known in the art, the invention makes use of these control laws to dynamically regulate complex in vivo and in vitro biochemical reactions and physiological processes. An example of biological control laws at work is the automatic self-directed, self-assembly of in vitro and in vivo clathrin and Coatomer proteins.

In one embodiment, intramolecular dynamics of biomolecules and the concerted and interlocking steps of conformational changes lead to deliberately purposeful actions. For example, one or more elements may fit spatially and each step in a process fits temporally (kinetically) with an element of anticipation of the purposeful outcome.

In another example case, the spatially and temporally defined events between the cell and one or more elements, for example, optical and or thermally induced conditions within an invention element and or a cell, may cause the invention element to release diagnostic and or therapeutic elements, and or monitoring agents to determine the most appropriate course of therapeutic action.

In one embodiment, the calculated utilization of biological control laws by one or more elements may, for example, provide for a sophisticated drug delivery system that provides optimal dosing by altering its drug delivery behavior, as well as producing minimal side effect profiles.

A further advantage of the invention is that it provides elements that can be bio-engineered to prevent in vivo uptake by one or more types of organs, tissue, cells, and bone. In the converse, another advantage is that one or more elements can be bio-engineered for highly selective uptake by one or more types of targeted cells, tissue, organs, bone, as well as by other organic and inorganic matter. In another embodiment, one or more elements comprise a non-selective uptake, non-targeted drug delivery system.

In another embodiment, the invention provides for the ability of one or more elements to intelligently monitor, control and regulate, react, and further adjust biological, other bio-chemical, or chemical processes after delivery of the payload, enabling precision actions, in vivo and or in vitro.

Another advantage of the invention is that clathrin can cross cell membranes including the blood brain barrier (Gragera et al 1993) and can move through the synaptic clefts (Granseth et al 2007). In one embodiment, bioengineered clathrin elements actively transport substances in and out of cells including neurons and blood brain barrier cells.

In another embodiment, one or more elements, operating alone or with one or more additional elements, comprise one or more types of cell membrane crossing elements and gain access to the cytosol and intracellular elements of one or more types, including one or more cell organelles. Such elements may, in one embodiment, require minimal functionalization to cross the cell membrane and or enter a cell organelle.

In one embodiment, one or more elements, in whole or in part, in one or more combinations, take one or more actions to create, spawn, comprise, modify, regenerate, reassemble, and or control and regulate one or more cells, cellular elements and or cellular processes of one or more types.

In one embodiment, one or more elements, in whole or in part, in one or more combinations, take one or more actions to rectify and or repair failures and defects in cellular processes, such as, endocytosis, exocytosis, mitosis, trafficking and signaling, and the like. Such failures and defects can lead to diseases, for example, cancer.

In one embodiment, one or more elements comprise in situ in vivo elements for remediation, removal and or sequestration of one or more types of contaminants, toxins, undesired organic or inorganic elements, biofilm, and the like.

In one embodiment, one or more elements comprise in situ environmental elements for remediation, removal and or sequestration of one or more types of in vitro environmental contaminants and or toxins; for example, chlorinated solvents TCE, PCE, PCBs, c-DCE, DNAPL, heavy metals (chromium), synthetic chemicals, and the like.

In one embodiment, some or all invention elements may also operate under the control and influence of other in vitro and or in vivo elements, including non-invention elements, and altogether may comprise a scalable, nanoscale platform.

In general, in another aspect, the invention is directed to a method of forming one or more types of scalable platforms, including the steps of providing one or more embodiments of the elements to deliberately carry out a series of tasks of one or more types, which tasks and or methods may be externally directed or internally self-directed, or a combination thereof. In other embodiments, one or more nanoscale platforms may be additionally composed of one or more non-invention elements and platforms of one or more types.

In one or more embodiments, an element can process, communicate, and store information in one or more types of operating environments, including, but not limited to, harsh environments of one or more types, e.g., radioactive, chemical, biological, and the like. One or more invention elements, in one platform embodiment, may also modify, process, manipulate, encode and decode, input, output, transmit, communicate, store and or read information using techniques and methods known in the art, in vivo and in vitro.

In one embodiment, one or more elements enable one or more types of information processing applications in one or more areas, including, but not limited to, one or more types of consumer, commercial, industrial, aerospace, and defense applications, which can include cryptography, cyber-defense, and the like.

In one embodiment, scalable information processing platforms use some or all invention elements or their effects as bits that are programmable into a plurality of logical states. In another configuration, the invention features a scalable information-processing platform that may include one or more elements.

As a general characteristic, one or more elements may take any suitable form, and multiple embodiments may be used as elements, and or further combined in any suitable manner to create one or more cargo carrying and or non-cargo carrying nanoscale elements (“elements”), and or multifunction nanoscale platforms (“platforms”) of one or more types, operating in vitro and or in vivo, such as: multiple polypeptide elements and platforms; biological elements and platforms; large molecule elements and platforms; small molecule elements and platforms; biomedical elements and platforms; medical elements and platforms; diagnosis, cure, mitigation, treatment, prevention of disease or other type of drug elements and platforms; targeted and or non-targeted delivery elements and platforms; cell, cell organelles, or cell material crossing elements and platforms; personal medicine elements and platforms; elements and platforms that, post administration, in whole or in part enter the central nervous system, including passing the blood brain barrier in 30 minutes or less and or in 30 minutes or more; healthcare elements and platforms; reproductive health elements and platforms; substance abuse disorder treatment elements and platform; bioengineered elements and platforms; cosmetic elements and platforms; agricultural elements and platforms; sensor elements and platforms; research and development elements and platforms; scientific elements and platforms; crystal elements and platforms; electronic elements and platforms; photonic energy elements and platforms; information processing or storage elements and platforms; energy storage elements and platforms; in situ elements and platforms for remediation, removal and or sequestration of undesirable elements and platforms of one or more types; quantum mechanical elements and platforms; quantum medicine platform, telecommunication elements and platforms; and the like; one or more of which nanoscale elements and platforms may be additionally composed of one or more non-invention elements and platforms of one or more types, and with or without one or more types of cargo elements located on and or in one or all or a subset of elements.

In general, in another aspect, the invention features a scalable platform that includes one or more embodiments of the elements described above.

In one embodiment, the scalable platform also includes an encoder for programming the bits (or qubits) of at least a subset of the elements, and a decoder for reading information from the bits (or qubits) of at least a subset of the elements.

In general, in another embodiment, an element and or a platform may be physically and/or functionally cooperative with other suitable types or forms of materials, substances, components, devices, or systems, in vitro and/or in vivo.

In general, in a further aspect, the invention is directed to a method of forming one or more formations of nanoscale elements formed in whole or in part in vitro from one or more isolated, synthetic and or recombinant amino acid residues comprising in whole or in part at least one or more types of clathrin and or coatomer I/II proteins, their constituent elements of one or more isoforms, including cloned isoforms, and or their accessory elements, which may further be composed of one or more types of metamaterials, forming one or more elements of one or more molecular weights, dimensions, geometries, symmetries, configurations and combinations; and, with or without one or more additional elements of one or more types located on and or in one or more elements; and, forming in whole or in part one or more types of element-carrying and or non-element-carrying nanoscale elements composed of one or more complete and or partial cage elements and structures; and, one or more of which elements may also comprise one or more non-invention elements of one or more types, forming hybrid elements; and, wherein one or more elements, using one or more types of methods, executes one or more functions and or effects one or more ends in vivo and or in vitro. In one embodiment, the method includes locating internally and or externally on one type of cage element at least one element capable of expressing one or more types of plasmonic states and or induced/produced types of effects.

In general, in another aspect, the invention is directed to a method of forming a scalable platform, including the steps of providing one or more embodiments of the elements described above, expressing one or more plasmonic states and or induced effects.

### BRIEF DESCRIPTION OF THE DRAWINGS

The foregoing and other aspects of the invention may be more fully understood from the following description, when read together with the accompanying drawings in which like reference numbers indicate like parts.

FIG. 1 is a conceptual diagram depicting a Clathrin triskelion comprised of one or more elements of one or more types employed in an illustrative embodiment of the invention.

FIG. 2 is a conceptual cross-sectional view of one or more Clathrin protein, receptor, adaptor protein, and cargo elements in an illustrative embodiment.

FIG. 3 is a computer generated frontal view of an actual Clathrin cage comprised of a plurality of Clathrin triskelia, and, in an illustrative embodiment, comprising one or more invention elements.

FIG. 4 is a flow diagram depicting conceptually the formation of individual Clathrin elements during endocytosis, which also serves to illustrate how the instant invention operates in one or more embodiments.

FIG. 5 is a conceptual diagram depicting Coatomer I/II protein comprised of one or more subunit and domain elements of the type employed in an illustrative embodiment of the invention.

FIG. 6 is an exemplary energy level diagram 600 illustrating the energy levels associated with a hyperfine interaction between electron and nuclear spin in the presence of magnetic fields.

### DESCRIPTION OF THE ILLUSTRATIVE EMBODIMENTS

The instant invention is composed of one or more formations of nanoscale elements, with at least some or all formed in vitro from one or more isolated, synthetic and or recombinant amino acid residues comprising in whole or in part at least one or more types of clathrin and or Coatomer I/II proteins of one or more isoforms, including cloned isoforms, forming one or more elements of one or more molecular weights, dimensions, geometries, symmetries, configurations and combinations, and which operate in vitro and or in vivo. In one invention embodiment, each of the elements is or includes a surface plasmon resonant element in one or more plasmonic states.

Plasmonic physics is an interdisciplinary field focusing on the unique properties of both localized and propagating surface plasmon polaritons (SPPs)—quasiparticles in which photons are coupled to the quasi-free electrons of metals. In particular, it allows for confining light in dimensions smaller than the wavelength of photons in free space, and makes it possible to match the different length scales associated with photonics and electronics in a single nanoscale device.

One or more embodiments of the current invention utilize plasmonics for biological sensing, sub-diffraction-limit imaging, medical therapies and diagnostics, focusing and lithography, and nano optical circuitry, to name some. In one or more example embodiments, but not limited to, plasmonics-based optical elements such as waveguides, lenses, beam splitters and reflectors are implemented by structuring metal surfaces or placing dielectric structures on metals, aiming to manipulate the two-dimensional surface plasmon waves, and also by using other plasmonic methods known in the art.

However, the abrupt discontinuities in the material properties or geometries of plasmonic elements can lead to increased scattering of SPPs, which significantly reduces the efficiency of these elements. In one or more embodiments, one or more methods known in the plasmonic art are used whereby SPP scattering can be substantially reduced, and even completely eliminated in the current invention. These SPP scattering reduction/elimination methods may include for example, but are not limited to: using properly designed anisotropic metamaterials; using transformation optics to route light at will by spatially varying the optical properties of a material; adiabatically tailoring the topology of a dielectric layer adjacent to a metal surface that enables a plasmonic Luneburg lens that can focus SPPs; enabling a plasmonic Eaton lens that can bend SPPs, and one or more other such methods as described in the literature or known in the art.

In one embodiment, an element provides electromagnetic radiation and energy transport at nanoscale dimensions, in vivo and or in vitro. In one embodiment, one or more plasmonic elements may induce one or more electromagnetic, photoelectric, thermal, quantum mechanical, and or other radiation, energies, states and effects, including lasing, in one or more dimensions, configurations and combinations. Alternatively, some of the elements are or include non-surface plasmon resonant elements. In another embodiment, an element provides cargo transport of one or more types of molecular elements, like a drug cargo element, in vivo and or in vitro. In one embodiment, one or more elements execute one or more functions and or effect one or more ends, in vivo and or in vitro, for example, but not limited to, a platform for one or more types of healthcare applications. In one embodiment, one or more elements form one or more configurations of one or more types, described below.

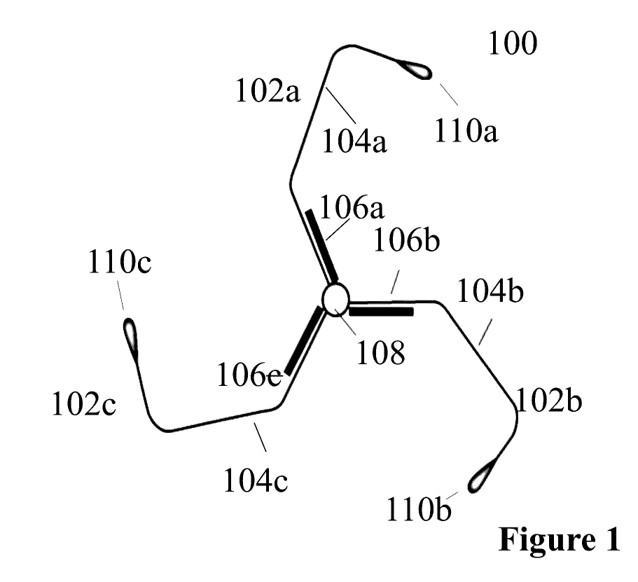

FIG. 1 is a conceptual diagram illustrating one type of partial cage element, the triskelion, which is the basic unit of a clathrin cage. It is a three-leg pinwheel protein structure, and each complete leg is typically called a ‘monomer’. The arrangement of the monomers in the three-dimensional protein is the quaternary structure. Each clathrin leg monomer is further composed of two subunits, one 190 kDa subunit (“heavy chain”) and one 24-27 kDa subunit (“light chain”). Three, two-subunit clathrin monomers self-assemble and combine to create triskelion element 100. It is this triskelion morphology that allows clathrin to form its unique polyhedral network.

In FIG. 1, the assembled triskelion element 100 is composed of three monomer leg elements 102a-102c. The three leg elements 102a-102c extend radially from a hub section 108. The filamentous portion of clathrin triskelion legs 102a-102c is formed by a continuous superhelix. A naturally occurring clathrin leg is about 47.5 nm (475 Å) long. In the instant invention, clathrin leg length and or molecular weights can be modified and or adjusted by using bioengineering techniques known in the art.

In the case of humans, there are two isoforms each of clathrin heavy chain (CHC17 and CHC22) and light chain (LCa and LCb) subunits, all encoded by separate genes. CHC17 forms the ubiquitous clathrin-coated vesicles that mediate membrane traffic. CHC22 is implicated in specialized membrane organization in skeletal muscle. CHC17 is bound and regulated by LCa and LCb, whereas CHC22 does not functionally interact with either light chain.

In one embodiment, a clathrin triskelion is composed of a trimer of heavy chains 104a-104c each bound to a single light chain 106a-106c, respectively. In the case of one isoform embodiment, CHC17, a clathrin heavy chain element is composed of a 1675 amino acid residue protein, which is encoded by a gene consisting of 32 exons. In the case of another isoform embodiment, CHC22, a clathrin heavy chain element is composed of a 1640 amino acid residue protein.

In one or more invention embodiments, elements formed in part from clathrin amino acid residues include, but are not limited to, a N-terminal globular domain 110a-110c (residues 1-494) that interacts with adaptor proteins (e.g., AP-1, AP-2, b-arrestin), a light chain-binding region (residues 1074-1552), and a trimerization domain (residues 1550-1600) near the C-terminus.

One or more of the clathrin heavy chain amino acid sequences and in whole or in part may be modified, altered, adapted or functionalized in one or more ways in one or more embodiments of the invention.

In the illustration, the three clathrin monomer elements 102a-102c are composed of six subunit elements, three of which subunits are the heavy chain subunit elements 104a-104c. The three heavy chain subunits are composed of several distinct domains and segments, one or more of which may comprise one or more invention elements in one or more embodiments, and may be functionalized via one or more techniques known in the art.

In general, each heavy chain comprises eight repeated motifs (CHCR 0-7), which make up the proximal, knee, distal and ankle segments of a clathrin leg. The heavy-chain amino terminus folds into the terminal domain (TD) and is attached to CHCR0 by a helical linker. (Brodsky, 2004). The three clathrin heavy chains are joined at their C-termini (located within hub element 108), extending into proximal and distal leg domains ending in globular N-terminal domain elements 110a-110c, and which are responsible for peptide binding. The clathrin heavy chain terminal domains provide multiple interaction sites for a variety of adaptor proteins (AP) that can bind multiple receptors occupied by ligands. These sites prevent chemical interactions between cargo elements. The heavy chain N-terminal domain elements 110a-110c are each composed of a seven-bladed beta-propeller connected to a flexible linker region, respectively. This propeller domain interacts with a host of accessory proteins participating in receptor-mediated endocytosis such as adaptor proteins, non-visual arrestins and the uncoating ATPase, hsc70. The propeller domain is followed by a long filamentous segment, which is interrupted by a bent region between the distal and proximal domains, and ends in the trimerization domain at the C-terminus.

Besides harboring determinants important for driving the association of individual clathrin molecules during lattice formation, each of the three heavy chain 104a-104c proximal domains also include binding sites for attaching the three light chain subunit elements 106a-106c, respectively, forming three complete clathrin monomers. The three light chain subunits are also composed of several distinct domains and segments, one or more of which may comprise one or more invention elements in one or more embodiments, and may be functionalized via one or more techniques known in the art.

Among other roles, clathrin light chains prevent clathrin heavy chains from interacting with each other. On the other hand, assembly proteins bind to light chains and cause a change in them such that they no longer prevent heavy chains from interacting. clathrin light chains consist of what has been described as a linear array of domains: regions of protein discernable from the primary sequence or with distinct biochemical properties. These are an N-terminal segment, a region that is 100% conserved between light chains, a portion to which Hsc70 binds, a calcium binding domain, a region which binds the heavy chain, a site for neuronal-specific splice inserts and then finally a calmodulin-binding domain at the C-terminus domain (Royle, 2006). The light chain C-terminal residues are also important for enhancing the in vitro assembly of hub 108 at low pH.

One or more of the clathrin light chain amino acid sequences and in whole or in part may be modified, altered, adapted or functionalized in one or more ways in one or more embodiments of the invention.

In one embodiment, each of the 3 heavy chain subunits 104a-104c may each have 3 light chains subunits 106a-106c attached, respectively, forming the typical, three-monomer clathrin triskelion structure. But in another embodiment, each leg 102a-102c may include only the 3 clathrin heavy chain subunits 104a-104c, respectively, which is distinctly unique from the classic clathrin monomer configuration. In yet another unique embodiment, only 3, non-attached light chain subunits 106a-106c are used.

In another distinctive embodiment of the invention, one or more elements of one or more types are formed from isolated, synthetic and or recombinant amino acid residues comprising in part one or more types of clathrin heavy chain and or light chain proteins of one or more isoforms.

In one embodiment, one or more N-terminal domain elements, e.g., 110a, 110b and or 110c are bioengineered to facilitate, modify, regulate or control peptide binding of one or more types, as well as interaction sites for one or more types of adaptor proteins.

In one embodiment, one or more domain elements of heavy chain subunits and or light chain subunits are bioengineered to facilitate, modify, regulate or control one or more clathrin protein characteristics and or behaviors in vivo and or in vitro.

In one embodiment, one or more clathrin triskelia elements of one or more types are coated in whole or in part with one or more types of metal molecules, e.g., noble, alkali, and or other suitable metals. In one embodiment, one or more metal coated clathrin triskelia elements are capable of expressing one or more plasmonic states and or effects when the triskelia element's surface plasmons are excited in a resonant manner. In other embodiments, one or more types of metamaterials are also used with triskelia elements to tailor an invention's output to specific conditions by changing the structural and dielectric parameters of the constituents.

In one embodiment, the dielectric constant for the interior of proteins that make up one or more clathrin and or coatomer elements are bioengineered via one or more methods known in the art. One or more embodiments will depend on the desired results and how much explicit averaging of protein conformations are included. Whereas a dielectric constant is a model for electrostatic relaxation, and if an embodiment includes this explicitly, the embodiment don't need a dielectric model. This topic and the methods required are extensively discussed in the literature.

In one embodiment, one or more types of triskelia protein elements in whole or in part comprise one or more dielectric elements of one or more types, one or more of which dielectric elements may be externally and or internally located with respect to the surface of one or more triskelia elements.

In one embodiment, one or more triskelia elements in whole or in part comprise one or more waveguide elements of one or more types, one or more of which waveguide elements may be externally and or internally located with respect to the surface of one or more triskelia elements.

In one embodiment, one or more clathrin triskelia elements of one or more types are used to carry one or more types of cargo, in vivo and or in vitro. In one embodiment, one or more clathrin triskelia composed of 3 heavy chain subunits 104a-104c, with or without 3 light chains subunits 106a-106c attached, compose one or more protein cage component elements that carry one or more cargo elements composed of and or coated in whole or in part with one or more types of molecules, e.g., noble, alkali, and or other suitable metals, and the cargo elements are capable in one or more embodiments of expressing one or more plasmonic states and types of electromagnetic radiation, including any energy aspects when the cargo element's surface plasmons are excited in a resonant manner. In other embodiments, one or more types of metamaterials are also used with one or more cargo elements to tailor an invention's output to specific conditions by changing the structural and dielectric parameters of the constituents.

In another embodiment, one or more cargo elements transported by one or more clathrin triskelia elements are non-plasmonic elements. In another embodiment, one or more cargo elements transported by one or more clathrin triskelia elements are exclusively non-plasmonic elements.

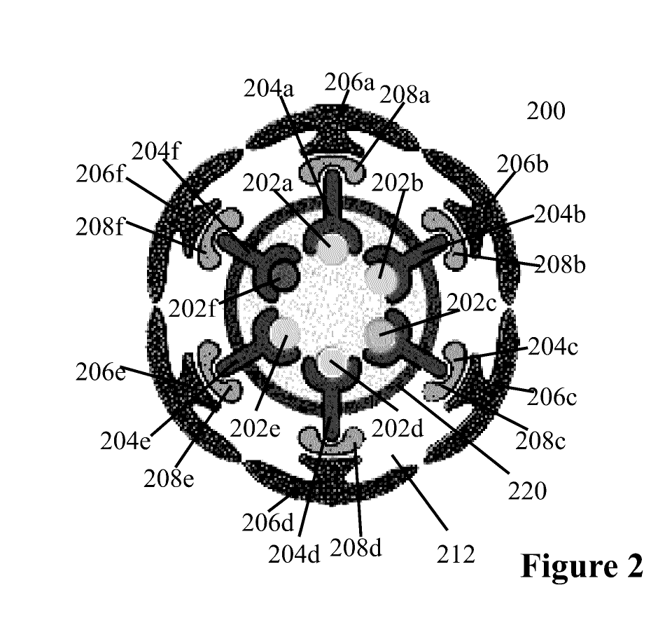

FIG. 2 is a conceptual cross-sectional view of an element 200, a complete cage element, according to one illustrative embodiment of the invention using clathrin protein. In one example embodiment, element 200 is composed of a plurality of protein molecules 206a-206f formed into a complete cage protein element 206. The protein molecules 206a-206f self-assemble in vitro to form the cage 200 that defines a cavity 212.

In one illustrative embodiment, the self-assembling protein molecules 206a-206f are clathrin molecules, and the clathrin cage 200 can be of any suitable size. According to the illustrative embodiment, the clathrin cage 200 has a diameter greater than about 20 nanometers. In various other illustrative embodiments, the clathrin cage 200 can have a diameter between about one nanometer and about fifty nanometers, a diameter between about fifty nanometers and about one hundred nanometers, or a diameter greater than about one hundred nanometers.

In another embodiment, an element 200 may also optionally include a vesicle or shell 220.

In one embodiment, one or more vesicle or shell 220 comprised of one or more types of molecules is encapsulated in whole or in part by cage 200, and vesicle/shell 220 may have any suitable size, such that its diameter is less than that of the clathrin cage 200.

In one embodiment, the vesicle/shell 220 may have any suitable geometry, including symmetrical or asymmetrical.

In one embodiment, one or more cage element 200 and or vesicle/shell element 220 are coated in whole or in part with one or more types of metal molecules, e.g., noble, alkali, and or other suitable metals. In one embodiment, one or more metal and or metal coated cage element 200 and or vesicle/shell element 220 are capable of expressing one or more plasmonic states and or effects when cage element 200's, and or vesicle/shell element 220's surface plasmons are excited in a resonant manner. In other embodiments, one or more types of metamaterials are also used with one or more cage element 200, and or vesicle/shell element 220 to tailor an invention's output to specific conditions by changing the structural and dielectric parameters of the constituents.

In one embodiment, one or more cage element 200 and or vesicle/shell element 220 in whole or in part comprise one or more dielectric elements of one or more types, one or more of which dielectric elements may be externally and or internally located with respect to the surface of one or more cage element 200, and or vesicle/shell element 220.

In one embodiment, one or more cage element 200 and or vesicle/shell element 220 in whole or in part comprise one or more waveguide elements of one or more types, one or more of which waveguide elements may be externally and or internally located with respect to the surface of one or more cage element 200 and or vesicle/shell element 220.

In one embodiment, one or more cage element 200 and or vesicle/shell element 220 of one or more types are used to carry one or more types of cargo elements 202a-202f, in vivo and or in vitro. In one embodiment, one or more cargo elements 202a-202f are composed of one or more types of molecules. In one embodiment one or more cargo elements 202a-202f, in whole or in part, are composed of and or are coated with one or more types of metals such as noble, alkali, and or other suitable metals. In one embodiment one or more cargo elements 202a-202f cargo elements are capable in one or more embodiments of expressing one or more plasmonic states and types of electromagnetic radiation, including any energy aspects when the cargo element's surface plasmons are excited in a resonant manner. In other embodiments, one or more types of metamaterials are also used with one or more cargo elements 202a-202f to tailor an invention's output to specific conditions by changing the structural and dielectric parameters of the constituents.

In one embodiment, cargo elements 202a-202f may also carry one or more additional cargo elements, like a metal element, drug element, and other suitable cargo.

In another embodiment, one or more cargo elements 202a-202f transported by one or more cage element 200 and or vesicle/shell element 220 are non-plasmonic elements.

In another embodiment one or more cargo elements 202a-202f are composed exclusively of one or more types of non-metal elements, for example, but not limited to, a drug element. In another embodiment, cargo element s202a-202f may be a mixture of metal and non-metal elements. In another embodiment, one or more cargo elements 202a-202f transported by one or more clathrin triskelia elements are exclusively non-plasmonic elements.

In another embodiment, one or more cargo elements 202a-202f may also comprise one or more types of complete and or partial shell or vesicle elements. In another embodiment, one or more cargo elements 202a-202f are composed exclusively of one or more types of non-shell/vesicle elements In one embodiment, one or more cargo elements are composed of a mixture of shell/vesicle and non-shell/vesicle elements.

In one embodiment, one or more cargo elements 202a-202f in whole or in part comprise one or more dielectric elements of one or more types, one or more of which dielectric elements may be externally and or internally located with respect to the surface of one or more cargo elements 202a-202f.

In one embodiment, one or more cargo elements 202a-202f in whole or in part comprise one or more waveguide elements of one or more types, one or more of which waveguide elements may be externally and or internally located with respect to the surface of one or more cargo elements 202a-202f.

In another embodiment, one or more cargo elements 202a-202f are attached externally to cage element 200. In another embodiment, one or more cargo elements 202a-202f are attached both internally and externally to cage element 200.

In another embodiment, an element 200 and or vesicle/shell 220 may optionally include a plurality of cargo positioning and attachment molecules 204a-204f composed of one or more types of molecules, for example, but not limited to, receptor molecules. In another embodiment, an element 200 and or vesicle/shell 220 may also optionally include a plurality of optional adapter molecules 208a-208f.

In another embodiment, adapter molecules 208 are bioengineered to recognize specific receptor molecules and to couple the receptor molecules to clathrin and or coatomer protein elements. In another embodiment, adapter molecules 208 can be natural, isolated, synthetic and or recombinant.

In one embodiment, one or more other elements, of one or more types, including invention and non-invention elements each may bond with one or more respective elements 200, 202, 220, and or 100 and its subcomponents.

As shown, in one embodiment, the optional positioning and attachment molecules 204a-204f each may bond with a respective cargo elements 202a-202fa-202f. In one embodiment using receptor molecules, the optional adapter molecules 208a-208f may bond the receptor molecules 204a-204f to the protein molecules 206a-206f, respectively. The bonding may be either covalent or non-covalent—the latter type including ionic interactions, hydrophobic interactions, or hydrogen bonds—depending on the application, system design, receptor design, cargo type and/or the interaction/application environment. Some G protein-coupled receptors (GPCRs) use covalent bonds, which are individually strong (e.g., it takes energy to break the covalent bond). In some instances, the clathrin molecule attaches covalently to the solution termini of alkanethiol SAMs/SPMs via covalent bonding. In other illustrative embodiments, electrostatic (ionic) bonding may be employed.

Most GPCRs do not form covalent bonds with their ligand when bound in the receptor. Noncovalent interactions are individually weak but collectively strong, such as with a substantial number of noncovalent interactions working together to hold a structure together, or a surface topography that enables substantial areas of two interacting surfaces to approach each other closely. Ligands generally bind to receptors via ionic, hydrophobic hydrogen and van Der Waal bonds, which often involve short-range interactions between molecules and the same molecule. These short-range non-covalent bond interactions and forces also underlie the intramolecular processes collectively referred to as conformational changes of proteins.

In one embodiment, also unlike prior clathrin art, a plurality of elements 206, with or without one or more additional other elements, comprise cage element 200, and element 200 has one or more elements, of one or more types and affixed via one or methods, located on the outside part of cage element 200; that is, located outside the cavity formed by cage 200. In another embodiment, a plurality of elements 206, with or without one or more additional other elements, comprise cage element 200, and element 200 has one or more elements, of one or more types and affixed via one or methods, located on both the outside, and inside parts (i.e., located within the cage cavity), of cage element 200.

According to one invention feature, cargo attachment element 204 and or element 208 shields cargo elements 202a-202f in the same element 200 from interacting.

According to another feature, the shielding properties of element 200 shields and inhibits chemical and molecular interactions between cargo element 202 and or vesicle element 200 and the external environment. According to a further feature, element 200 protectively sequesters cargo elements 202a-202f and or vesicle element 200 from the external environment.

Cage 200 and or triskelia element 100 and subcomponents areas formed from isolated, synthetic and or recombinant amino acid residues comprising in whole or in part one or more types of clathrin. In one or more embodiments, the optional positioning and attachment elements 204a-204f can be naturally occurring or formed from isolated, synthetic and or recombinant amino acid residues in whole or in part to recognize specific cargo elements 202a-202f. Likewise, the optional adapter molecules 208a-208f can be naturally occurring or formed from isolated, synthetic and or recombinant amino acid residues in whole or in part to recognize and couple to particular positioning and attachment molecules 204a-204f.

In another embodiment, one or more non-invention, “natural” clathrin elements 206b-206f (the term “natural” hereinafter generally refers to non-isolated, non-recombinant, and non-synthetic protein elements) join with one or more isolated, recombinant, and or synthetic elements; in this example, 206a; to form a natural/invention hybrid clathrin cage element 200. In another embodiment, hybrid cage element 200 may also be composed of natural element 220, which is a vesicle, forming a hybrid clathrin coated vesicle.

In one embodiment, the protein cage 200 forms to enclose (e.g., to “coat”) a vesicle or shell 220 within the cavity 212. ARF-GTP, appropriate lipids, and cytosolic factor(s) are used for AP-1 clathrin coated vesicle assembly. Recruitment of AP-1 (Assembly Polypeptides) onto liposomes is ARF-dependent and facilitated by cytosolic ARF Guanine Nucleotide-Exchange Factor (GEF). Lipid composition is important and modulates ARF and AP-1 binding. The vesicle or shell 220 can be formed, for example from naturally occurring membrane material, such as L-2-Phosphatidylinositol-4, 5-bisphosphate or from synthetic membrane materials, such as a fully synthetic liposome like one containing DOPC DOPE cholesterol or from a mixture of both, for example, from synthetic lipids such as L-2-Phosphatidylcholine (PC) from soybeans containing 20% PC (Sigma P5638).

In another embodiment, one or more non-invention, non-clathrin elements 206b-206f join with one or more isolated, recombinant, and or synthetic elements; in this example, 206a; to form a hybrid clathrin cage element 200. In another embodiment, a hybrid cage element 200 may also be composed of vesicle or shell element 220, forming a hybrid clathrin coated vesicle.

In other embodiments, vesicle/shell 220 may be formed in whole or in part from micelles, from silica shells, from isolated, synthetic and or recombinant amino acid residues of one or more types. In other embodiments, vesicle or shell 220 may be formed in whole or in part from one or more suitable metal molecules that support one or more types of plasmonic actions and electromagnetic emission, including any energies.

In other embodiments, vesicle or shell 220 in whole or in part may be formed from and or coated in whole in one or more suitable metal molecules that support one or more types of plasmonic actions and electromagnetic emission, including any energies.

In other embodiments, one or more dielectric elements of one or more types that enable one or more types of plasmonic actions and electromagnetic emission, including any energies in whole or in part are externally and or internally formed, structured and or supported by vesicle or shell 220.

In other embodiments, one or more metamaterial elements of or more types of that enable the tuning or adjustment of one or more types of plasmonic actions and electromagnetic emission, including any energies in whole or in part are externally and or internally formed, structured and or supported by vesicle or shell 220.

In one embodiment, optional adapter molecules may tether the optional vesicle or shell 220 to the cage 200. The adapter molecules 208a-208f, in turn, bond to optional receptor molecules 204a-204f disposed around the periphery of the vesicle or shell 220. According to the illustrative embodiment, the receptor molecules or the positioning and attachment molecules 204a-204f extend through the vesicle or shell 220 to capture the cargo elements 202a-202f. In another embodiment, the optional vesicle or shell 220 is free floating within the cage 200. In another embodiment, one or more optional vesicle or shell 220 elements are externally located on the cage 200. In another embodiment, one or more optional vesicle or shell 220 elements is both externally and internally located with respect to the cage 200.

In one embodiment, cargo elements 202a-202f are cavity forming and are non-permeable, semi-permeable, and or permeable, and or can change from one permeable state to another.

In another embodiment, vesicle or shell 220 is cavity forming and non-permeable, semi-permeable, or permeable, and or can change from one permeable state to another.

In another embodiment, vesicle or shell 220 may be located on one or more triskelion elements 100 and its subcomponents. In another embodiment, vesicle or shell 220 may be located on one or more cage element 200.

In one embodiment, but not limited to, one or more elements 208 of the instant invention may comprise the major types of adaptor elements, like the heterotetrameric adaptor protein (AP) elements, and the monomeric GGA (Golgi-localizing, Gamma-adaptin ear domain homology, ARF-binding proteins) adaptors. In one illustrative embodiment, elements 208 comprise one or more small sigma subunits of various adaptins from different AP adaptor elements. The AP complex family has six members in mammals: AP-1A, AP-2, AP-3A and AP-4 are ubiquitously expressed. The other two members, AP-5 and AP-6, are cell-type specific isoforms of AP-1A and AP-3A: the epithelium-specific AP-1B and the neuron-restricted AP-3B. (Ohno, 2006). In another embodiment, AP180, like AP-2 and AP-3, binds to N-terminal domains 110a-110c of clathrin. In one embodiment, one or more AP elements may be functionalized at one or more heavy chain terminal domain elements 110a-110c.

In one embodiment, one or more cargo elements 202a-202f, of one or more types, including invention and non-invention elements each may directly attach and or bond with one or more complete and or partial cage elements 200.

In one embodiment, one or more cargo elements 202a-202f, of one or more types, including invention and non-invention elements each may directly attach and or bond with one or more triskelion elements 100. In another embodiment, one or more cargo elements 202a-202f may be located on one or more triskelion subunits, e.g., on a clathrin monomer.

In one embodiment, triskelia element 100 and its subcomponents, cage element 200, cavity element 212, cargo elements 202a-202f, and or vesicle or shell element 220 comprise one or more types of electromagnetic radiation and energy transport elements.

In one embodiment, triskelia element 100 and its subcomponents, cage element 200, cavity element 212, cargo elements 202a-202f, and or vesicle or shell element 220 is capable of expressing one or more surface plasmon resonance states. In another embodiment, triskelia element 100 and its subcomponents, cage element 200, cavity element 212, cargo elements 202a-202f, and or vesicle or shell element 220 is capable of expressing a plurality of surface plasmon resonance states.

In one embodiment, triskelia element 100 and its subcomponents, cage element 200, cavity element 212, cargo elements 202a-202f, and or vesicle or shell element 220 comprise and or utilize one or more types of plasmonic elements that enable one or more types of plasmonic actions, effects, electromagnetic radiation emission and or energies.

In one embodiment, triskelia element 100 and its subcomponents, cage element 200, cavity element 212, cargo elements 202a-202f, and or vesicle or shell element 220 contain and or transport one or more types of metals that enable one or more types of plasmonic actions, electromagnetic radiation emission, and or any energies.

In one embodiment, triskelia element 100 and its subcomponents, cage element 200, cavity element 212, cargo elements 202a-202f, and or vesicle or shell element 220 are coated at least partially or completely in one or more types of metals that enable one or more types of plasmonic actions, electromagnetic radiation emission, and or any energies.

In one embodiment, triskelia element 100 and its subcomponents, cage element 200, cavity element 212, cargo elements 202a-202f, and or vesicle or shell element 220 comprise in whole and or in part one or more elements that emit surface plasmon enhanced electromagnetic radiation, including any other energies, of one or more types; and further, triskelia element 100 and its subcomponents, cage element 200, cavity element 212, cargo elements 202a-202f, and or vesicle or shell element 220 may be additionally composed in whole and or in part of one or more types of elements that comprise one or more inner layers and or outer layers composed of one or more types of materials, including one or more layers that may further be comprised of metal surface elements of one or more types; and further, triskelia element 100 and its subcomponents, cage element 200, cavity element 212, cargo elements 202a-202f, and or vesicle or shell element 220 may be additionally composed in whole and or in part of one or more types of elements that comprise one or more elements formed and defined by structuring one or more metal layer surface elements; and further, triskelia element 100 and its subcomponents, cage element 200, cavity element 212, cargo elements 202a-202f, and or vesicle or shell element 220 may be additionally composed in whole and or in part of one or more types of elements that may further comprise one or more elements formed and or defined by placing one or more types of structure elements on one or more metal layer surface elements; wherein one or more layers are configured to completely surround, and or to cover at least some part of, one or more other layers, and at least one portion of one or more layers emits electromagnetic radiation of one or more types, including any energies, when one or more appropriate types of energies is applied to one or more layers.

In one embodiment, triskelia element 100 and its subcomponents, cage element 200, cavity element 212, cargo elements 202a-202f, and or vesicle or shell element 220 comprise and or utilize one or more types of internally and or externally located stimuli, such as, but not limited to, light source and or energy source elements that enable and or excite one or more types of plasmonic actions, electromagnetic radiation emission, and or any energies.

In one embodiment, triskelia element 100 and its subcomponents, cage element 200, cavity element 212, cargo elements 202a-202f, and or vesicle or shell element 220 comprise one or more collections of non-coherent and or coherent light source elements, and or other collections of energies.

In one embodiment, surface plasmons are composed of electron oscillations that allow electromagnetic radiation and or energies to be localized, confined, and or guided on sub-wavelength scales via triskelia element 100 and its subcomponents, cage element 200, cavity element 212, cargo elements 202a-202f, and or vesicle or shell element 220.

In one embodiment, triskelia element 100 and its subcomponents, cage element 200, cavity element 212, cargo elements 202a-202f, and or vesicle or shell element 220 comprise and or utilize one or more types of dielectric elements, including dye or dopant molecules as appropriate, that support one or more types of plasmonic actions.

In one embodiment, triskelia element 100 and its subcomponents, cage element 200, cavity element 212, cargo elements 202a-202f, and or vesicle or shell element 220 comprise and or utilize one or more types of waveguides, which may be internally and or externally tunable to one or more conditions, that support one or more types of plasmonic actions, electromagnetic radiation emission, and or any energies.

In one embodiment, one or more methods are used for externally stimulating and or inducing local deformation of one or more elements, including triskelia element 100 and its subcomponents, cage element 200, cavity element 212, cargo elements 202a-202f, and or vesicle or shell element 220, to support one or more types of plasmonic actions.

In one embodiment, triskelia element 100 and its subcomponents, cage element 200, cavity element 212, cargo elements 202a-202f, and or vesicle or shell element 220 comprise and or utilize one or more types of interfaces and or boundary layers, which also may be internally and or externally tunable to one or more conditions, between a metal and or metamaterial element and a dielectric element.

In another illustrative embodiment, triskelia element 100 and its subcomponents, the cargo elements 202a-202f, the vesicle or shell element 220, and or cage 200 include and or comprise a plasmonic element of one or more types. In one illustrative plasmonic embodiment, one or more metal elements of one or more geometries and comprised of one or more types of molecules, and in whole or in part comprise and or are encapsulated within cargo elements 202a-202f, vesicle or shell 220, and or within cavity 212 of cage 200. In one embodiment, one or more types of dielectric elements, with or without dyes, additive scattering particles, and the like, may also be carried within a cavity forming, non-permeable vesicle or shell 220 and or cargo elements 202a-202f. In one embodiment cage 200 encapsulates vesicle or shell 220, and or carries cargo elements 202a-202f. In another embodiment, cage 200, cavity 212, vesicle/shell 220 and or element 100 is surrounded by one or more dielectric elements that encapsulate, permeate and or saturate in whole or in part the cage 200, cavity 212, vesicle/shell 220 and or element 100.

In one embodiment, one or more triskelia element 100 and subcomponents, cargo elements 202a-202f, vesicle or shell element 220, and or cage 200 are positioned and located at one or more distances, above, below, and or within one or more other types of other elements, for example, but not limited to, a glass element, a metal element, a polycarbonate element, a polymer element, a semiconductor element, a biological element, and the like.

In one embodiment, one or more triskelia element 100 and or its subcomponents, cargo elements 202a-202f, vesicle or shell element 220, and or cage 200 act as a biomolecular device at the nanoscale that interacts with light in the form of surface plasmons and can confine photons within very small spaces. Trapping and sustaining light in these ultrasmall bio-nanoscale elements creates extreme conditions in which the interaction of light and matter is strongly altered.

In one embodiment, one or more triskelia element 100 and or its subcomponents, cargo elements 202a-202f, vesicle or shell element 220, and or cage 200 comprise one or more types of plasmonic elements, which also may include a bio-nano-spaser element. A spaser which is the equivalent of a laser, but it amplifies plasmon resonances rather than photons, with plasmonic resonators instead of a resonant cavity. Here the high-quality factor needed for lasing and amplification is a feature of the collective “coherent” response of the whole array.

In another embodiment, and in contrast to conventional lasers operating at wavelengths of suitable natural molecular transitions, a lasing bio-nano-spaser does not require an external resonator.

In one embodiment, plasmonic electromagnetic radiation emissions and or laser spaser emission wavelength can be controlled by metamaterial designs.

In one embodiment, a bio-nano-spaser is a quantum amplifier of surface-plasmon emission.

In one embodiment, at one or more specific wavelengths of the optical pumping source, plasmon resonance phenomena occur within triskelia element 100 and its subcomponents, cargo elements 202a-202f, the vesicle or shell element 220, and or cage 200. Conductive electrons in their encapsulated metal elements, excited at such wavelengths, oscillate in phase with the incident energies, and strongly enhance the electromagnetic field at the interface between the metal element and dielectric element, producing surface plasmon waves with large amplitude. Light in a gap size at 1 nanometer or greater than 1 nanometer is feasible in one embodiment. In one embodiment, a plasmonic element of one or more types may be as small as one nanometer.

In one embodiment, the light in a plasmonic element bounces around on the surface of one or more types of a metal and or metal coated triskelia element 100 and subcomponents, cargo elements 202a-202f, vesicle or shell element 220, and or cage 200, one or more of which elements may comprise one or more dimensions, geometries, symmetries, configurations and combinations, in the form of plasmons. In one embodiment, light from the plasmonic element can remain confined as plasmons or it can be made to leave one or more bio-nanoparticle's surface as photons in the visible-light range. A plasmonic element must be “pumped” to supply the necessary energy. This pumping is accomplished by bombarding the bio-nanoparticle with pulses of light that may be external and or internal to triskelia element 100 and subcomponents, cargo elements 202a-202f, the vesicle or shell element 220, and or cage 200. In another embodiment one or more types of nanoscale optical pumping sources are carried onboard triskelia element 100 and subcomponents, cargo elements 202a-202f, the vesicle or shell element 220, and or cage 200 and or be in close proximity.

Optical pumping of the plasmonic element and excitation of surface plasmon resonance is done through methodologies known in the art. In one embodiment, the energy source for the plasmonic mechanism is an active (gain) medium that is excited externally and or internally to triskelia element 100 and its subcomponents, vesicle or shell 220, cargo elements 202a-202f and/or cage 200. This excitation field may be optical and unrelated to the plasmonic element's operating frequency; for instance, a plasmonic element can operate in the near infrared but the excitation of the gain medium can be achieved using an ultraviolet pulse.

In one embodiment, the current invention provides a room temperature, ultralow-threshold, highly controllable, strongly directional, ultra-bright laser light source device that operates at the nanoscale, and also features the capability to store light.

Outgoing light from the triskelia element 100 and its subcomponents, vesicle or shell 220, cargo elements 202a-202f and/or cage 200 can be directed and controlled through one or more methodologies also known in the art.

In one example embodiment, switching gain/lasing of a plasmonic element by local deformation is a feature the current invention exploits for various effects.

In one illustrative embodiment, a highly controllable plasmonic element is a regulated source of photons for use in quantum computing and quantum cryptography.

In another illustrative embodiment, a highly controllable plasmonic element with broad and continuous tunable wavelength and ultra low threshold is a light source for use in wavelength division multiplexing, optical fiber communications, and free-space optical interconnects.

In one embodiment, a highly controllable plasmonic element with broad and continuous tunable wavelength is a light source for use in ultra bright, ultra low power human- and machine-readable displays.

In another embodiment, a plasmonic element is used for light and/or information storage.

Another embodiment of a highly controllable plasmonic element constitutes an ultra bright ultra low power light source for use in medical diagnosis, therapy, and prosthesis, in vivo and/or in vitro.

In another illustrative embodiment, a plasmonic element constitutes broad and/or narrow spectrum sensors.

In another illustrative embodiment, a plasmonic element of one or more types utilizes solar energy as an excitation and or optical pumping source for use in high efficiency solar cells.

In one embodiment, plasmonic element with strongly directional ultra bright light output are a source of highly steerable and directed photonic energy.
1. In another embodiment, one or more element 100, 200, 202, 206, 220, bioengineered proteins and or with lipids, together with one or more plasmonic elements and one or more types of associated electromagnetic radiation, including any energy elements are used to perform the basic properties necessary for the functioning of nano-biomolecular electronic devices. In one approach, biological materials 100, 200, 202, 206, and or 220 conduct and transfer molecules from one location to another, are capable of major color changes on application of an electric field or light, and can produce cascades that can be used for amplification of an optical or an electronic signal. All these properties can be applied to electronic switches, gates, storage devices, biosensors, biological transistors, to name just a few.
2. In general, the electrical properties of bilayer lipid membranes are easily measurable for signal generation and transduction. In one embodiment, 100, 200, 202, 206, and or 220, comprise hybrid elements, which, when interacting with intact plasma membranes can be considered to act as tiny capacitors under the influence of an electric field generated by one or more plasmonic elements and one or more types of associated electromagnetic radiation, including any energy elements. In one embodiment, whereas sufficiently high field strength may increase the membrane potential past a critical point leading to the breakdown of the membrane, experimental, care is taken. (Dielectric breakdown of biological membrane occurs at about 1 volt across the membrane.) On the other hand, in one embodiment, the use of electrostatic potentials around the lipid molecules is implemented, because they are controllable.

In another embodiment plasmon-emitted electromagnetic radiation and or laser spaser emissions and or local tissue heating cause agents trapped in one or more thermally sensitive cargo elements 202a-202f, vesicle or shell 220, and or cage 200 to be triggered and released, thereby forming a targeted agent delivery system. Diagnostic and therapeutic agents may be simultaneously delivered via this site-specific delivery embodiment by using one or more thermally sensitive cage 200, cargo elements 202a-202f, and or vesicle or shell 220.

In another embodiment, one or more element 100, 200, 202, 206, and or 220 produce and or induce one or more types of states and or effects caused by their coming into contact with a particular metabolic state, medical disorder, disease pathology, genotype, phenotype and or other specific stimuli. In another embodiment, one or more agents are thereby selectively triggered and released from elements 100, 200, 202, 206, and or 220. In doing so, they form a targeted agent delivery system without exposing the entire body—or an indiscriminate area—to a similar dose of one or more types of states and or effects, and or cargo agents. The agents may be delivered in vivo by means known in the art.

In one illustrative embodiment, one or more types of states and or effects operate on elements 100, 200, 202, 206, and or 220 that may carry one or more materials, such as, but not limited to, therapeutic, diagnostic, and or therapeutic agents within an aqueous interior, and or that may have one or more entrapped nanoparticles such as liposomes, micelles, proteins, other biological and or bioengineered elements, including organic, inorganic, and synthetic materials, and or that may have one or more hydrophobic materials bound to a lipid bilayer membrane.

In one thermal energy embodiment, the well-known permeability increase at the phase transition temperature provides a means to trigger release of an agent in locally heated tissues, like a therapeutic agent for example, carried by elements 100, 200, 202, 206, and or 220.

In one embodiment, efficient in vivo or in vitro release of entrapped agents at non-targeted and or targeted sites are triggered by light emitted by one or more elements when the elements 100, 200, 202, 206, and or 220 contain a photoisomerisable species.
3. In one embodiment, one or more types of luminescent elements provide optical pumping sufficient to excite one or more in vivo and or in vitro plasmonic elements and one or more types of associated electromagnetic radiation, including any energy elements.
4. In an illustrative embodiment, in vivo release of one or more elements composed of one or more liposomal-entrapped and or non-liposomal-entrapped agents is accomplished by photons emitted by plasmonic elements and one or more types of associated electromagnetic radiation, including any energy elements, which light may be coherent and or non-coherent in nature. In one illustrative embodiment, one or more plasmonic elements and one or more types of associated electromagnetic radiation, including any energy elements produce specific light wavelength emissions caused by their coming into contact with, for example, a specific disease at in vivo target site. A photosensitive delivery system composed of diagnostic, therapeutic, and or prosthetic agents is thereby triggered, and the agents are released. In one embodiment, this configuration comprises a highly targeted drug delivery system. For example, in one embodiment, one or more elements comprise an amphipathic lipid, such as a phospholipid, having two chains derived from fatty acid that allow the lipid to pack into a bilayer structure. One or more photosensitizers may be incorporated into the entrapped materials' cavity and or membranes.

In an illustrative embodiment, in vivo and or in vitro release from one or more elements 100, 200, 202, 206, and or 220 of one or more agents is optically triggered by photons emitted by one or more elements. In one illustrative embodiment, one or more elements produce specific light wavelength emissions caused by their coming into contact with, for example, a specific disease at in vivo target site and causes diagnostic, therapeutic, and or prosthetic agents contained in a photosensitive delivery system to be triggered and released from 100, 200, 202, 206, and or 220, thereby forming a highly targeted drug delivery system. For example, in one embodiment, 100, 200, 202, 206, and or 220 contain an amphipathic lipid, such as a phospholipid, having two chains derived from fatty acid that allow the lipid to pack into a bilayer structure. One or more photosensitizers may be incorporated into the entrapped materials' cavity and or membranes.

In one illustrative embodiment, a phospholipid (1,2-(4′-n-butylphenyeazo-4″(-phenylbutyroyl))-glycero-3-phosphocholine (Bis-Azo PC′), substituted with azobenzene moieties in both acyl chains that can be photoisomerised by a fast plasmonic element pulse. One or more other photoisomerisable species can be used in other embodiments. Agent release from elements 100, 200, 202, 206, and or 220 occurs on the milliseconds timescale. One or more photosensitised elements 100, 200, 202, 206, and or 220 thereby serve as light sensitive elements to allow for the triggered release of agents from elements 100, 200, 202, 206, and or 220. In one embodiment, cholesterol additives may be used. The addition of cholesterol may have a marked effect on kinetics of agent release and in some circumstances can result in substantial enhancement of light sensitivity in photosensitised elements 100, 200, 202, 206, and or 220. In another embodiment, thermal and photosensitive activation systems acting together comprise one or more elements.

In one embodiment, an invention element varies plasmon resonant wavelengths of one or more elements 100, 200, 202, 206, and or 220, which may be a metal and or metal coated, and provides a method for spectrally-coding their light-mediated content release, so that the release event is initiated by the specific wavelength of light used to illuminate the one or more elements. In one embodiment, an element, spectrally coded release enables applications in controlled delivery of multiple agents to support complex diagnostic tests and therapeutic interventions. In another embodiment, the resonant peak of these metal and or metal coated vesicles is spectrally tunable in the near infrared range by varying the concentration of metal, e.g., noble, alkali, and or other suitable metals, deposited on the surface of vesicles. In other embodiments, one or more types of metamaterials can be also be used to tailor an invention element's output to specific conditions by changing the structural and dielectric parameters of the constituents.

In another embodiment, thermal and photosensitive activation systems are packaged and contained in the same element 100, 200, 202, 206, and or 220.

In another embodiment, one or more elements in one or more element 100, 200, 202, 206, and or 220 operates in an intelligently staged sequence or orchestrated series of actions, which may be multiplexed by using one or more light and or thermal energy emitting sources and or done in parallel by using one or more light and or thermal energy emitting sources, such that various optical and or thermal energy from one or more elements operate on one or more photosensitive and or thermal sensitive element 100, 200, 202, 206, and or 220 that contain one or more agents, resulting in a staged series of overall actions that follow an intelligently ordered sequence of events. For example, first a diagnostic agent from an element 100, 200, 202, 206, and or 220 is released by an optical and or thermal trigger and the agent's positive finding of a disease, like cancer or HIV then causes one or more therapeutic agents to be released from the same element and or another element by one or more optical and or thermal triggers. Agent dosages are released in calculated amounts, and the dosages may be non-targeted and or targeted.

In one illustrative embodiment, one or more element 100, 200, 202, 206, and or 220, in whole or in part, form a photonic energy-based therapeutic and or diagnostic device at the nanoscale, i.e., a nanoscale low level laser light therapy (LLLT) system, which may also comprise diagnostic properties and thereby enable a novel type of theranostic element.

The use of low levels of visible or near-infrared (NIR) light for reducing pain, inflammation and edema, promoting healing of wounds, deeper tissues and nerves, and preventing tissue damage has been known for almost forty years since the invention of lasers. Originally thought to be a peculiar property of laser light (soft or cold lasers), the subject has now broadened to include photobiomodulation and photobiostimulation using non-coherent light.

There are perhaps three main areas of medicine and veterinary practice where LLLT has a major role to play. These are (i) wound healing, tissue repair and prevention of tissue death; (ii) relief of inflammation in chronic diseases and injuries with its associated pain and edema; (iii) relief of neurogenic pain and some neurological problems.

Despite many reports of positive findings from experiments conducted in vitro, in animal models and in randomized controlled clinical trials, LLLT remains controversial. This likely is due to two main reasons; firstly, the biochemical mechanisms underlying the positive effects are incompletely understood, and secondly, the complexity of rationally choosing amongst a large number of illumination parameters such as wavelength, fluence, power density, pulse structure and treatment timing has led to the publication of a number of negative studies as well as many positive ones. In particular, a biphasic dose response has been frequently observed where low levels of light have a much better effect than higher levels.

LLLT (low level laser light therapy, including phototherapy and photostimulation) has been shown to modulate biological processes, depending on the power density, wavelength, and frequency, and to have positive effects on wound healing, on improving angiogenesis, on muscle regeneration and diabetic wounds repair Moreover, the histological analysis of tissue indicates that laser irradiation shortens the inflammatory phase as well as accelerating the proliferative and maturation phase, and positively stimulates the regeneration of injured epidermis and the reparation of injured striated muscle. The pioneering work of Tiina Karu has defined critical parameters in this rapidly growing area governing wavelengths, output power, continuous wave or pulsed operation modes, pulse parameters, coherence and polarization, and has also indicated possible biological light acceptors at organic, cellular, subcellular and molecular level On the basis of these extensive studies it has been proposed that the terminal enzyme of the respiratory chain cytochrome c oxidase located in mitochondria acts as photoacceptor for the red-to-near IR region in eukaryotic cells, and the modulation of the redox state of the mitochondria generates secondary reactions through cell signalling molecules.

LLLT is transduced to a cellular signal, e.g., the structure of mitochondria act as waveguides that capture, direct and transduce photons to chemical energy. Note that mitochondria participate intimately in cell death

Still, low-power laser therapy is used by physical therapists to treat a wide variety of acute and chronic musculoskeletal aches and pains, by dentists to treat inflamed oral tissues and to heal diverse ulcerations, by dermatologists to treat edema, non-healing ulcers, burns, and dermatitis, by orthopedists to relieve pain and treat chronic inflammations and autoimmune diseases, and by other specialists, as well as general practitioners. Laser therapy is also widely used in veterinary medicine (especially in racehorse-training centers), and in sports-medicine and rehabilitation clinics (to reduce swelling and hematoma, relieve pain, improve mobility, and treat acute soft-tissue injuries). Lasers and LEDs are applied directly to the respective areas (e.g., wounds, sites of injuries) or to various points on the body (acupuncture points, muscle-trigger points).

In 2002, MicroLight Corp received 510K FDA clearance for the ML 830 nm diode laser for the treatment of carpal tunnel syndrome. There were several controlled trials reporting significant improvement in pain, and some improvement in objective outcome measures. Since then several light sources have been approved as equivalent to an infrared heating lamp for treating a wide-range of musculoskeletal disorders with no supporting clinical studies.

The beneficial effect of LLLT on wound healing can be explained by considering several basic biological mechanisms including the induction of expression cytokines and growth factors known to be responsible for the many phases of wound healing. Firstly, there is a report that HeNe laser (632.8 nm) increased both protein and mRNA levels of IL-1 and IL-8 in keratinocytes. These are cytokines responsible for the initial inflammatory phase of wound healing. Secondly, there are reports that LLLT can upregulate cytokines responsible for fibroblast proliferation and migration, such as bFGF, HGF and SCF. Thirdly, it has been reported that LLLT can increase growth factors such as VEGF, responsible for the neovascularization necessary for wound healing. Fourthly, TGF-β is a growth factor responsible for inducing collagen synthesis from fibroblasts, and has been reported to be upregulated by LLLT. Fifthly, there are reports that LLLT can induce fibroblasts to undergo transformation into myofibloblasts, a cell type that expresses smooth muscle-actin and desmin, and has the phenotype of contractile cells that hasten wound contraction.

Studies from Whelan's group have explored the use of 670 nm LEDs in combating neuronal damage caused by neurotoxins. Among the wavelengths tested (670, 728, 770, 830, and 880 nm), the most effective ones (670 nm and 830 nm) paralleled the NIR absorption spectrum of oxidized cytochrome c oxidase.

Animal models have been employed to study LLLT effects in nerve repair. Byrnes et al. [56] used 1,600 J/cm2 of 810-nm diode laser to improve healing and functionality in a T9 dorsal hemisection of the spinal cord in rats. Anders et al. studied LLLT for regenerating crushed rat facial nerves; by comparing 361, 457, 514, 633, 720, and 1064 nm, and found the best response with 162.4 J/cm2 of 633 nm HeNe laser.

Although LLLT is mostly applied to localized diseases and its effect is often considered to be restricted to the irradiated area, there are reports of systemic effects of LLLT acting at a site distant from the illumination. It is well known that UV light can have systemic effects, and it has been proposed that red and NIR light can also have systemic effects. These have been proposed to be mediated by soluble mediators such as endorphins and serotonin.

Laser treatment increases energy production in the brain also holds promise for enabling Parkinson's nerve cells to work better and have a reduced chance of dying. The interesting characteristic about laser light is that it can penetrate the scalp and skull directly, activating brain tissues without having to expose the brain surgically. Preferably, this laser treatment would be delivered in vivo to the precise site of action. In one embodiment, one or more invention elements, using in vivo targeting elements known in the art, are delivered to the site of action to provide a neuroprotective effect.

Prior research has focused on the effect of pulsed light laser irradiation vis-à-vis two distinct biological effects: neurite elongation under nerve growth factor (NGF) stimulus on laminin-collagen substrate and cell viability during oxidative stress. It has been shown that laser irradiation affects the in vitro maturation of that pheochromocytoma cell line 12 (PCl2) cells by stimulating NGF-induced neurite elongation on a laminin-collagen coated substrate. Moreover, coherent light irradiations showed a protective effect on oxidative stress induced by H₂O₂. Study results demonstrated that 670 nm laser light treatment was neuroprotective and stimulates neural maturation, thus providing additional evidence that red-near-IR light might represent a potential, novel, non-invasive, therapeutic intervention for the treatment of numerous diseases.

Whereas laser light has been shown to combat neuronal damage caused by neurotoxins, as well as promoting neuroprotective effects, e.g., but not limited to, Parkinson's, in one embodiment, one or more invention elements provide LLLT in vivo to one or more neuronal sites of action afflicted by one or more types of central nervous system (CNS) disorders, and may additionally carry cargo elements to further enhance neuroprotective and or therapeutic effects, forming a nanoscale element unique in the art.

In one embodiment, invention delivered neuroprotective elements are composed of brain-derived neurotrophic factor (BDNF), which has been consistently shown to modify excitatory synaptic transmission and long-term synaptic plasticity in a variety of preparations (Lessmann et al. 1994; Kang & Schuman, 1995; Levine et al. 1995; Figurov et al. 1996; Akaneya et al. 1997; Carmignoto et al. 1997; Gottschalk et al. 1998; Huber et al. 1998; Messaoudi et al. 1998). The proposed mechanisms underlying the actions of BDNF include the immediate and long-term modulation of both pre- and postsynaptic function (Poo, 2001). Regarding presynaptic function at CNS synapses, BDNF rapidly increases the frequency of spontaneous miniature excitatory postsynaptic currents (mEPSCs), without affecting their amplitude or kinetics (Lessmann et al. 1994; Carmignoto et al. 1997; Lessmann & Heumann, 1998; Li et al. 1998a; Schinder et al. 2000). In addition, BDNF increases the variance of evoked EPSC amplitudes (Lessmann & Heumann, 1998), modulates paired-pulse facilitation, and attenuates synaptic fatigue during high-frequency stimulation (Figurov et al. 1996; Gottschalk et al. 1998). Mice with a constitutive deletion of the BDNF gene exhibit several presynaptic impairments, including pronounced synaptic fatigue, fewer docked vesicles, and reduced expression levels of synaptobrevin and synaptophysin (Pozzo-Miller et al. 1999), two vesicle proteins involved in their mobilization and docking (Sudhof, 2004). In addition, direct measurements of glutamate concentration have also confirmed that BDNF enhances K+-evoked transmitter release from cultured neurons and isolated synaptosomes (Numakawa et al. 1999; Jovanovic et al. 2000). Lastly, using restricted genetic deletions it was demonstrated that BDNF is selectively required for a form of long-term potentiation (LTP) of synaptic strength that recruits a presynaptic module of expression (Zakharenko et al. 2003). Taken together, these observations provide consistent evidence that BDNF modulates the efficiency of vesicular release during exocytosis of excitatory neurotransmitter.

In one embodiment, one or more invention elements provide in vivo site directed LLLT, with or without additional cargo elements of one or more types, e.g., BDNF, but not limited to, enables neuroprotective effects in one or more regions of the brain adversely affected by depression, drug addiction, post traumatic stress, traumatic brain injury, stroke, coma, and the like.

In one embodiment, one or more invention elements provide in vivo site directed LLLT, with or without additional drug cargo elements of one or more types, in addition to providing photobiomodulation and photobiostimulation using non-coherent light. This is an embodiment unique in the art.

In one embodiment, one or more invention elements provide in vivo site directed LLLT, with or without additional drug cargo elements of one or more types, in addition to utilizing the inherent cell signaling properties of clathrin invention elements for enhanced therapeutic effect.

In another illustrative embodiment, one or more invention elements, in whole or in part form a photodynamic therapy (PDT) system.

In one PDT embodiment for cancer treatment, a drug or dye is administered to the patient either intravenously or by injection. The drug travels through the blood stream and localizes on cancer cells. After an appropriate time (24-78 hours) the localized drug is activated with one or more invention elements. The drug destroys the cancer cells, while leaving the normal tissue intact.

In one PDT embodiment, drugs are localized on cancer cells, and this singlet oxygen reacts with the cancer cell, killing it. The accepted mechanism for PDT involves the interaction of an excited state of the drug or dye with the ground state of oxygen. A molecule of the drug absorbs a photon of red light and is excited to the first excited singlet state. If this singlet state is long lived energy can be transferred from the singlet state to the triplet state through interstitial crossing. This triplet state can react with local oxygen molecules created by an excited state of oxygen called singlet oxygen. Singlet oxygen is cytotoxic and destroys nearby cells.

There may be one of two mechanisms by which singlet oxygen can attack a cell, according to the type of PDT embodiment. In one PDT embodiment, the drug is localized on the outside of the cell membrane, and the singlet oxygen destroys the micro vascular of the cell, and the cell dies due to lack of oxygen. In another PDT embodiment, the dye is left for a longer period of time before light activation, and the dye may permeate the cell membrane and attack the mitochondria of the cell, leading to programmed cell death or apoptosis.

In a two-photon PDT embodiment, multi-photon excitation is made possible by using the high peak power of a femtosecond laser source. Two-photon excitation allows low average power IR energy to be used for excitation. This results in two key benefits: 1) deeper penetration depth of light, 2) new PDT treatments of skin melanoma without the addition of a drug or dye.

Verteporfin photodynamic therapy (PDT) is the most effective treatment for age-related macular degeneration, using laser activation of a photosensitizing dye to achieve closure of choroidal neovascularization. Although PDT preferentially affects pathologic vessels, it can also cause collateral damage to the overlying retina. In one study, it was found that the neuroprotective agent brain-derived neurotrophic factor (BDNF) reduces this retinal damage. In one study, it was found that the neuroprotective agent brain-derived neurotrophic factor (BDNF) reduces this retinal damage. These results suggest that adjunctive neuroprotective therapy in the form of BDNF may reduce collateral damage to photoreceptors and improve visual outcome after PDT. In one embodiment, one or more invention elements deliver PDT to the local site of action for treating age-related macular degeneration, and additionally transport one or more BDNF cargo elements to reduce collateral damage to photoreceptors and improve visual outcome after PDT.

In another illustrative embodiment, one or more elements may be a light source for use in a photodynamic therapy (PDT) system for age related macular degeneracy (AMD).

In one embodiment, one or more elements are used to remediate biofilms. Biofilms that form in the human body are up to ten thousand times more resistant to antibiotics and immune systems than free-floating bacteria, making them very difficult to treat medically. In humans, and animals biofilms are responsible for 80% of all infections. In agriculture, every year billions of dollars of crops are lost due to the formation of biofilms. Industrial needs for effective biofilm dispersion include surface coatings and cleansing products. In the commercial and industrial space, biofilms are a multibillion-dollar global problem that ranges from causing biofouling of energy production and distribution systems.

In one embodiment, one or more elements are used to remedy in vivo biofilms in humans, animals, and plants. By one estimate, in the energy industry, biofilms are implicated in a wide range of petroleum process problems, from the production field to the gas station storage tank. In the field, sulfate reducing biofilm bacteria produce hydrogen sulfide (soured oil). In the process pipelines, biofilm activity develops slimes that impede filters and orifices. Biofilm and biofilm organisms also cause corrosion of pipeline and petroleum process equipment. These problems can be manifested throughout an oil or gas production facility to the point where fouling and corrosive biofilm organisms have even been found on the surfaces of final product storage tanks. Methods commonly employed to prevent biofilm formation include chemical treatment of the water column by biocides or coating the surfaces with antifouling paints. As these methods invariably lead to pollution, environmentally friendly methods are desirable.

Lasers are known to cause bacterial mortality. In one embodiment, one or more laser spaser elements comprise an intelligent, “rifle shot” approach that selectively targets, disrupts, and eliminates biofilm, in vitro and or in vitro, and used at a very early stage it would yield significant performance benefits. For example, it has been shown that with marine biofilms, low-power pulsed laser irradiation for a very short duration can remove a significant portion of biofilm from various types of surfaces.

In addition, plasmon electromagnetic radiation emissions and or low level laser spaser therapy may show an ability to modulate cellular metabolism and alter the transcription factors responsible for gene expression which may, in one embodiment, alter gene expression, cellular proliferation, intra-cellular pH balance, mitochondrial membrane potential, generation of transient reactive oxygen species and calcium ion level, proton gradient, and consumption of oxygen.

In one or more embodiments, highly targeted, plasmon electromagnetic radiation emissions and or laser spaser irradiation may induce genetic alterations of biofilms and thus could lead to a breakthrough approach for removing biofilms and disrupting their growth, in vivo and in vitro, in humans, animals, and plants, as well as in the industrial and commercial space. In one or more embodiments, one or more elements achieve biofilm removal and disruption, and additionally, may cause very early disruption of quorum sensing, a type of collective decision-making process used by decentralized groups of biofilm bacteria to coordinate their behavior, thereby preventing biofilm from becoming a highly organized biohazard.

In one or more embodiments, one or more elements safely operate in a wide variety of difficult and harsh environments. Invention elements are composed of environmentally safe clathrin protein coated vesicles, and can be safely used in vivo and in vitro for detecting and destroying one or more types of chemicals, toxins, biological agents, radioactive elements, and other environmental elements, as well as biofilm type infections.

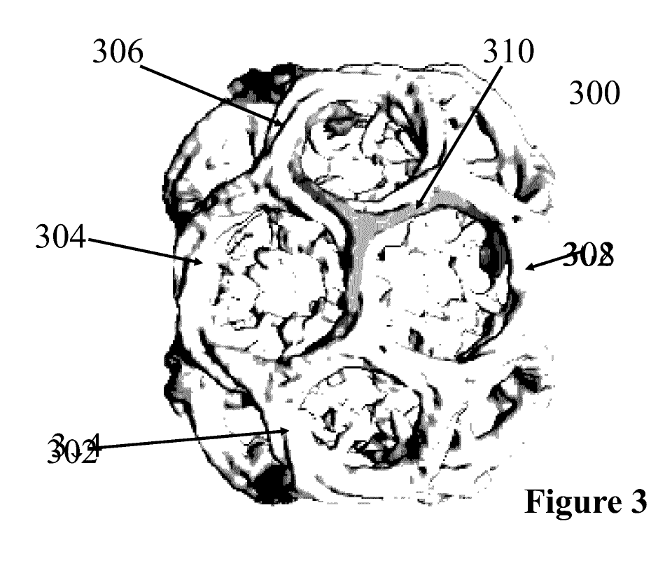

FIG. 3 is a computer generated frontal view of a clathrin cage 300 composed of a plurality of natural clathrin triskelia elements 302-308, respectively. In an illustrative embodiment, element 310 is an invention element, composed of three heavy chain elements 104a-104c—which may or may not include three respective light chain elements 106a-106c—forming a hybrid or fused cage 300 composed of natural elements and invention elements. In this role, element 310 comprises an efficacious replacement for a natural triskelia element, whose properties are transformed by one or more invention elements.

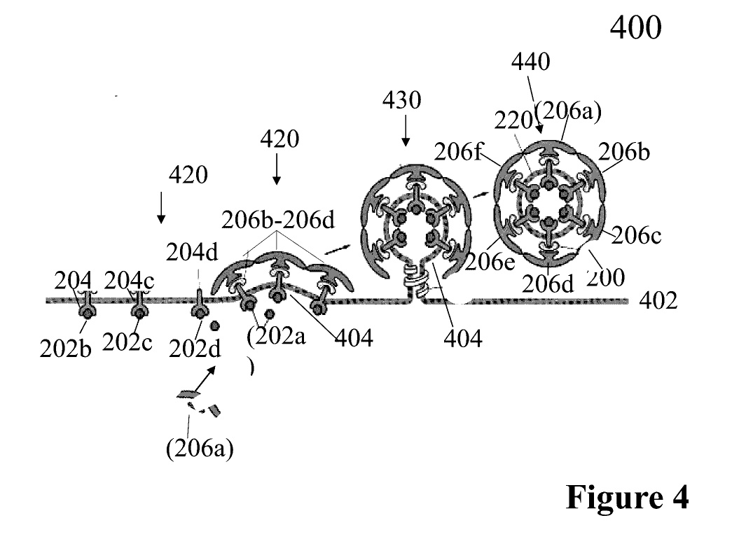

FIG. 4 is a flow diagram 400 depicting, conceptually, the formation of a plurality of natural clathrin elements 206b-2026f, and, in this example, along with invention element (206a) into cage 200, which at step 440, shows clathrin coated vesicle or shell 220. The process by which natural clathrin molecules 206b-206d obtain natural cargo molecules 202b, 202c, and 202d in this example is known as clathrin mediated endocytosis (CME), a process wherein a cell takes in macromolecules by forming vesicles derived from the plasma membrane. Endocytosis is crucial to cellular function. Via CME, cells internalize cargo attachment elements, transmembrane channels, transporters and extracellular ligands such as hormones, growth factors and nutrients.

In one embodiment, one or more invention elements induce and or perform one or more actions that prompt, create, spawn, comprise, modify, repair, regenerate, reassemble, and or control and regulate CME, as well as exocytosis, mitosis, trafficking, signaling processes, other behaviors, and the like. Defects and disorders in any of these critical cellular processes can lead to disease, and one or more types of these processes may be modified in one or more embodiments of the instant invention, for example, to achieve therapeutic effect.

In one embodiment, one or more invention elements take and or induce one or more types of actions relating to cell signaling that are efficacious, and involving receptor-mediated endocytosis that encompass nutrient uptake (LDL, transferrin, etc.), membrane recycling, membrane protein recycling, antigen uptake, synaptic vesicle recycling, and signaling receptor down-regulation.

In one or more embodiments, one or more plasmonic elements and one or more types of associated electromagnetic radiation, including any energy elements and their effects functionally serve as a drug element; e.g., they interact in one or more ways with cells and their processes, and by so doing diagnose, regulate and or cure one or more diseases and disorders.

An increase of a cellular component is called upregulation. Upregulation is an increase in the number of receptors, e.g., see elements 204b, 204c, and 204d in FIG. 4, on the surface of target cells, making the cells more sensitive to a hormone or another agent. For example, there is an increase in uterine oxytocin receptors in the third trimester of pregnancy, promoting the contraction of the smooth muscle of the uterus. In one or more embodiments, one or more invention elements, either by acting alone and or in part with other elements of one or more types, and one or more elements efficaciously modify, control and regulate, interfere with, create, and or spawn elements, and or induce actions or behaviors that increase the upregulation of one or more types of receptors of the surfaces of target cells.

On the other hand there is downregulation, an example of which is the cellular decrease in the number of receptors to a molecule, such as a hormone or neurotransmitter, which reduces the cell's sensitivity to the molecule. In the literature, downregulation is the process by which a cell decreases the quantity of a cellular component, such as RNA or protein, in response to an external variable. In one or more embodiments, one or more plasmonic elements and one or more types of associated electromagnetic radiation, including any energy elements, either by acting alone and or in part with other elements of one or more types, including natural and or non-invention elements, and efficaciously modify, control and regulate, interfere with, create, and or spawn elements, and or induce actions or behaviors that increase the downregulation of one or more types of receptors.

Exocytosis is the reverse process of endocytosis, whereby a cell directs secretory vesicles out of the cell membrane. These membrane-bound vesicles contain soluble proteins to be secreted to the extracellular environment as well as membrane proteins and lipids that are sent to become components of the cell membrane. Exocytotic vesicles are usually not clathrin-coated; most of them have no coat at all. However, two observations suggest that clathrin effectively ‘tracks’ vesicle proteins leaving a synapse. In one study (Granseth, et al, 2008) the amount of a clathrin light chain (LC) tagged with the element mRFP leaving the synapse was proportional to the number of vesicles released by the stimulus, as assessed by the amplitude of a sypHy signal (sypHy is an improved fluorescent reporter of exocytosis). Second, in the same study the movement of LC-mRFP began without a significant delay and peaked with the sypHy signal. The movement of clathrin out of the synapse together with synaptophysin and synaptobrevin is most easily explained as representing CME (clathrin mediated endocytosis) of vesicles at sites removed from the active zone. This interpretation is consistent with studies showing that the machinery for CME is not at the active zone, but in the surrounding regions of membrane (Heuser & Reese, 1973; Ringstad et al. 1999; Qualmann et al. 2000; Teng &Wilkinson, 2000). Thus, clathrin is naturally found in the extracellular space and may play a role in regulating exocytosis and or endocytosis. In one illustrative embodiment, one or more invention elements efficaciously operate in inter- and or extra-cellular spaces of one or more types; for example, can perform remediation, sequestration, or removal of one or more types of undesirable elements.

Membrane trafficking only occurs during interphase. As the cell enters mitosis, clathrin-mediated membrane traffic is rapidly shut down and only resumes in late telophase. clathrin may therefore have a separate function that is distinct from membrane trafficking, which operates during mitosis. clathrin is thus a multifunction protein: during interphase its function is in membrane trafficking and during mitosis it has a role in stabilizing spindle fibers (Royle, 2006). In one embodiment, mitosis may be efficaciously controlled and regulated, modified, and or induced via one or more invention plasmonic elements and their multidimensional electromagnetic radiation.

In another embodiment, one or more plasmonic elements, including any energy elements are composed of, but not limited to, one or more isolated, synthetic, and or recombinant adaptor protein molecules, tubulin protein molecules, dynamin protein molecules, epsin protein molecules, endophilin protein molecules, synaptotagmin protein molecules, and or other types of protein molecules associated with clathrin and Coatomer proteins and processes, for enhanced efficacious effect.

In another embodiment, one or more natural adaptor protein molecules, tubulin protein molecules, dynamin protein molecules, epsin protein molecules, endophilin protein molecules, synaptotagmin protein molecules, and or other types of protein molecules involved with associated with clathrin and Coatomer proteins and processes form efficacious hybrid elements when also composed of one or more types of invention plasmonic elements.

The CME process involves a dynamic interaction between clathrin and a wide range of other protein molecules, and altering the compositions and behaviors of the various molecular parties involved. For example, the cell uses endocytosis to control and regulate the density of receptors on the cell surface and to acquire nutrients. Endocytosis of ligand-activated cargo attachment elements is essential for the proper attenuation of a variety of signal transduction processes, as well as for co-localization of activated cargo attachment elements with downstream signaling molecules. Endocytosis also counterbalances secretion, preventing continuous expansion of the plasma membrane. Endocytosis thus internalizes macromolecules and fluid, and after sorting, directs the internalized molecules for degradation or recycling.

The endocytosis process begins when proteins bound to cargo attachment elements accumulate in coated pits 404, which are specialized regions of the cell membrane 402 where it is indented and coated on its cytoplasmic side with a bristle-like coat composed of two natural proteins: clathrin and protein adapters. Most, if not all, intracellular transport vesicles are encased in a proteinaceous coat, one class of which is clathrin-coated vesicles (CCVs). CCVs also mediate the transport of lysosomal hydrolases from the trans-Golgi network, as well as the efficient internalization of extracellular solutes such as nutrients, hormones, growth factors, and immunoglobulins at the plasma membrane.

Clathrin also transports proteins from the Golgi to other organelles. In neurons, endocytosis is critical to allow rapid synaptic vesicle regeneration. Besides clathrin, there are other coat-forming proteins, such as COP I and COP II, which mediate intracellular traffic and there are clathrin-independent endocytic pathways which mediate internalisation of a variety of cargo (Royle, 2006).

In one embodiment, the natural endocytosis process is transformed via one or more plasmonic elements and one or more types of associated electromagnetic radiation, including any energy elements, whereby endocytosis may be efficaciously controlled and regulated, modified, and or induced by one or more electromagnetic aspects into a versatile therapeutic method to regulate the intensity, localization, half-life and function of signaling elements (signalosomes) that form in cells upon, for example, binding of growth factors, cytokines and morphogens to their cognate receptors. In one example embodiment, a plasmonic element rectifies breakdowns in the function of endocytic adaptors that might facilitate impairment of tissue homeostasis and consequent tumor development. In another illustrative embodiment, one or more plasmonic elements and one or more aspects of electromagnetic radiation or actions interact with natural adaptor proteins required for appropriate receptor downregulation and which play distinct roles in oncogenesis. (Crosetto, et al. 2005) In another embodiment, CME elements might also comprise one or more invention cargo elements (202a in FIG. 4), which can be drugs, other ligands, and the like.

In one embodiment, referring to FIG. 4, a natural clathrin coated vesicle or shell 220 is desired to form to endocytose over-expressed natural receptor elements 204b and 204c that are initially located outside cell membrane 402. The appearance of one or more types of plasmonic elements, such as element (206a) in the illustrative example, outside cell membrane 402 and or by crossing 402, and using one or more plasmonic elements and one or more types of associated electromagnetic radiation, including any energy elements and or effects prompt, create, spawn, mediate, control and regulate, regenerate, and or interact with one or more natural endocytosis processes and behaviors. Via the prompting of one or more plasmonic elements and one or more types of associated electromagnetic radiation, including any energy elements, one or more biological processes acting on cell membrane 402 induce a clathrin bud 404 to form at 420.

As shown at 430 and 440, after forming completely around bud 404, natural clathrin elements 206b-206d pinch off (scission) from membrane 402 with the desired over expressed receptors 204b and 204c held inside vesicle or shell 220. After excision, bud 404 has evolved into a plurality of natural clathrin elements 206b-206f, some of which are attached to one or more types of over expressed receptor elements 204b and 204c, as well as attached to other receptor elements; which in this example are the normally expressed natural elements 204d.

In one illustrative embodiment, one or more plasmonic elements follow normal pathways within the cell, cause downregulation of the desired over-expressed receptor elements, which may be associated with one or more types of neurotransmitters, viruses, cholesterol, as well as with other cargo types, restoring a cell to its normal, healthy state.

In another illustrative embodiment, clathrin coated vesicle structure 440 in FIG. 4 is composed of vesicle invention element 400 and a naturally occurring clathrin vesicle, and produce efficacious properties and behaviors.

In another embodiment, one or more hybridized invention elements enter the cell nucleus and or other organelles and cell elements.

In one embodiment, one or more elements 100, 200, 206, 204, and or 208 operate alone without cargo elements 202a-202f, and comprise one or more types of inherently efficacious elements, of one or more types, like a drug element, for example.

The fusion and or participatory actions or effects of one or more plasmonic elements and one or more types of associated electromagnetic radiation, including any energy elements 206a, 204a, and or 208a in FIG. 2, may yield a therapeutic effect, and are an example embodiment of adjunctive, inherently efficacious invention elements in action. In another embodiment, natural or hybrid CCV 440 in FIG. 4 also includes one or more invention cargo molecules (202a) that may have been transported into the cell via their attachment to one or more natural and or invention receptor elements.

Referring again to FIG. 4, in another example embodiment, a therapeutic effect is accomplished via one or more plasmonic elements and one or more types of associated electromagnetic radiation, including any energy elements that regulate EGFR (epidermal growth factor receptor). EGFR exists on the cell surface and is activated by binding of its specific ligands including epidermal growth factor and transforming growth factor a (TGFa). When these natural cargo attachment elements are activated, cells rapidly clear them from the surface and destroy them. Control of EGF receptor signaling is performed by clathrin-mediated endocytosis. Natural clathrin coats also exist on endosomes and are involved in endosomal sorting of the EGFR. A defect in this overall process will likely lead to uninhibited growth of cells and tumors. EGFR expression, over-expression, or mutation is associated with cancer progression, advanced disease, drug resistance, aggressive disease, poor prognosis, and reduced survival. EGFR is considered one of the main proteins elevated in breast, lung, and prostrate cancers, among others. Brain cancer is also implicated with over-expressed EGFR. Other work has shown that using monoclonal antibodies for EGFR, or anti-EGFR, has proven an effective strategy for getting nanoparticles to specifically attach themselves to cancer cells. Additional work has shown effectiveness of EGFR as the cancer-targeting pathway. In one embodiment, one or more types of participatory actions of one or more plasmonic elements and one or more types of associated electromagnetic radiation, including any energy elements 100, 200, and or 206 may yield a therapeutic effect in controlling, regulating, or mediating EGFR activity. In another example embodiment of modulating EGFR activity, cargo elements (202a) in FIG. 4 may also comprise one or one or more types of cancer drugs or biologicals delivered directly into cells and organelles that are transported into the cell along with a plasmonic/electromagnetic component. In another embodiment, invention cargo elements (202a) may comprise one or more diagnostic agents, or combine one or more diagnostic agents and therapeutic agents in the same plasmonic/electromagnetic payload. In one or more embodiments, one or more plasmonic elements and one or more types of associated electromagnetic radiation, including any energy elements of one or more types may thus comprise an efficacious method for the diagnosis, treatment, remedying, curing, and or prevention of one or more types of cancers, including those cancer types that fall outside the scope of EGFR-related activity.

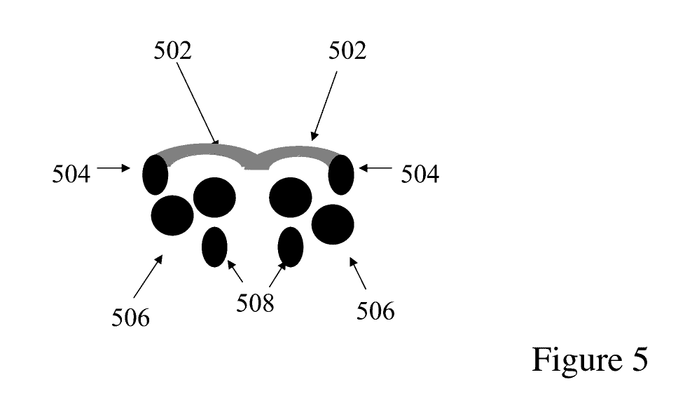

FIG. 5 is a conceptual diagram illustrating the basic units of Coatomer I and II proteins. COPII and clathrin cages are both constructed from ∂-solenoid and β-propeller building blocks (Fotin et al., 2004b; ter Haar et al., 1998; Ybe et al., 1999). In various embodiments of the invention, one or more elements of one or more types are formed from isolated, synthetic and or recombinant amino acid residues comprising in whole or in part one or more types of Coatomer proteins of one or more isoforms, including cloned isoforms. In another embodiment, one or more Coatomer subunit amino acid sequences may be modified, altered, adapted or functionalized in one or more ways in one or more embodiments of the invention.

In one embodiment, Coatomer is composed of seven distinct subunits: alpha, beta, beta′, gamma, delta, epsilon and zeta subunits, respectively.

In clathrin, a triskelion assembly unit lies at each vertex, and the ∂-solenoid legs of neighboring triskelia interdigitate extensively as they extend toward the adjacent vertices; the β-propeller is not part of the architectural core and instead projects in toward the membrane to interact with adaptor molecules (Fotin et al., 2004; Kirchhausen, 2000). In contrast, the COPII assembly unit is a rod that constitutes the edge of a cuboctahedron, and four rods converge to form the vertex with no interdigitation of assembly units. ∂-solenoid domains form the core of the edge, but, unlike clathrin, the COPII vertices are formed from β-propellers. In summary, the COPII and clathrin lattices seem not to share common construction principles other than the use of ∂-solenoid and β-propeller folds.

Crystallographic analysis of the Coatomer II assembly unit reveals a 28 nm long rod, element 502, comprising a central solenoid dimer capped by two B propeller domains, elements 504, at each end. GTPase, elements 508, bind to adaptor elements 506, which bind to elements 502. In the illustration, element 502a is an invention element that acts as an efficacious replacement element for one or more natural element 502, forming a hybrid Coatomer element. The structural geometry and properties of COPI coats remain to be determined. However, by analogy to the COPII and clathrin structural units, they probably involve a preassembled cage protein (CP) scaffold that is generated by the β-propeller-containing and ∂-solenoid-containing subunits and an adaptor protein (AP) subcomplex. Together these could form an AP-CP heptaheteromeric functional unit in the cytosol. (Gurka, et al. 2006)

COPI and COPII play a major role in exocytosis, as also can their invention element counterparts. clathrin can also play a role in exocytosis, but to a lesser extent than Coatomer. The exocytosis process refers to the fusion of intracellular vesicles with the plasma membrane. It occurs via two major processes, a constitutive pathway and a regulated pathway. These are the major ways that the cell secretes materials, wherein a cell secretes macromolecules (large molecules) by fusion of vesicles with the plasma membrane. Coatomer-coated vesicles, which are typically less than fifty nanometers in size, are also involved in vesicular transport between the Golgi apparatus, endoplasmic reticulum and plasma membrane. Coatomer I vesicles shuttle elements from the Golgi to the endoplasmic reticulum (ER). Coatomer II vesicles shuttle elements from the ER to the Golgi. Coat-protein I/II subunits (COPs) require ATP to assemble into a coat and unlike clathrin coats, the Coatomer coat remains on the vesicle until docking occurs. In some instances, Coatomer proteins are also involved in endocytosis, but are unrelated to clathrin. Thus, while clathrin also mediates endocytic protein transport from the ER to the Golgi, Coatomers (COPI, COPII) primarily mediate intra-Golgi transport, as well as the reverse Golgi to ER transport of dilysine-tagged proteins. Coatomers reversibly associate with Golgi (non-clathrin-coated) vesicles to mediate protein transport and for budding from Golgi membranes. In one or more embodiments, one or more COPI/COPII and or clathrin invention elements and their one or more types of associated electromagnetic radiation, including any energy elements, either by acting alone and or in part with other elements of one or more types, including natural and or non-invention elements, efficaciously modify, control and regulate, interfere with, create, and or spawn elements and or induce actions or behaviors involving exocytosis.

Cells of the mammalian immune system undergo selective changes in protein glycosylation during differentiation, immune activation, and autoimmune disease. In many, if not most of these types of diseases endocytosis and cellular trafficking and signaling plays a role. Referring again to FIGS. 1, 2, 3, 4, (and 5, in some embodiments), in one embodiment, but not limited to, one or more plasmonic elements and one or more types of associated electromagnetic radiation, including any energy elements in whole or in part selectively interfere with, fuse with, control and regulate, induce, and otherwise modify endocytosis, receptor-specific processing, trafficking and signaling, and other behaviors for efficacious effect in one or more types of autoimmune diseases, including, but not limited to, one or more types of diabetes, CNS autoimmune diseases, and other types of autoimmune diseases that effect the body.

Referring again to FIGS. 1, 2, 3, 4, (and 5 in some embodiments), in one embodiment, but not limited to, one or more plasmonic elements and one or more types of associated electromagnetic radiation, including any energy elements selectively interfere with, control and regulate, and or modify secretory products that participate in inflammation and immunoregulation; and also in other embodiments, whereby endocytosis mediated by specific receptors for immunoglobulin or by other opsonins is important in removal of damaged self or foreign particles. In another embodiment, defects in membrane receptor function, whether inherited or acquired, and the pathogenesis of immune diseases may be remedied, inhibited, mitigated, and or prevented.

Referring again to FIGS. 1, 2, 3, 4, and 5, in one embodiment, but not limited to, one or more plasmonic elements and one or more types of associated electromagnetic radiation, including any energy elements of one or more types efficaciously fuse with and or functionally replace one or more natural elements commonly found in endocytosis, exocytosis, mitosis, trafficking and signaling, and the like; either by acting alone and or in part with other elements of one or more types, including natural and or non-invention elements. In one embodiment, one or more invention element effects are utilized to produce an efficacious outcome.

Referring again to FIGS. 1, 2, 3, 4, and 5, but not limited to, in another embodiment, one or more plasmonic elements efficaciously enter a cell, its elements, and or its organelles, such as its nucleus, either by acting alone and or in part with other elements of one or more types, including natural and or non-invention elements. In one embodiment, one or more invention element effects are utilized to produce an efficacious outcome.

Referring again to FIGS. 1, 2, 3, 4, and 5, in another embodiment, but not limited to, one or more plasmonic elements and one or more types of associated electromagnetic radiation, including any energy elements efficaciously create, spawn, comprise, modify, repair, regenerate, reassemble, and or control and regulate one or more natural elements commonly found in endocytosis, exocytosis, mitosis, trafficking and signaling, other cellular behaviors, and the like, either by acting alone and or in part with other elements of one or more types, including natural and or non-invention elements.

Referring again to FIGS. 1, 2, 3, 4, and 5, in another embodiment, but not limited to, one or more plasmonic elements and one or more types of associated electromagnetic radiation, including any energy elements efficaciously interact with natural and or genetically engineered elements to encode components of the intracellular sorting machinery that mediate the selective trafficking of lipids and proteins in the secretory and endocytic pathways, to efficacious effect.

Referring again to FIGS. 1, 2, 3, 4, and 5, in another embodiment, but not limited to, one or more plasmonic elements and one or more types of associated electromagnetic radiation, including any energy elements efficaciously interact with genetic agents and elements, including, but not limited to, proteins; peptides; DNA and DNA variants; RNA and RNA variants such as mRNA, iRNA and siRNA; RNA-induced silencing complex (RISC), other genetic-modifying agents and methods, and the like.

In another embodiment, but not limited to, one or more plasmonic elements and one or more types of associated electromagnetic radiation, including any energy elements efficaciously interact with one or more oligonucleotides in antisense therapy. These antisense DNA drugs work by binding to messenger RNAs from disease genes, so that the genetic code in the RNA cannot be read, stopping the production of the disease-causing protein.

In another illustrative embodiment, one or more plasmonic elements may further comprise one or more RNAi (RNA interference) elements and or RNAi variants such as small interfering RNA molecules (siRNA), but not limited to, that may efficaciously interact with proteins in the cell. In one embodiment they also may form a nanoscale element called a RISC (RNA-Induced Silencing Complex) with electromagnetic properties. RNAi and or RISCs may be used to head off a genetic disease before the first symptom appears, based on an analysis of an individual's predisposition to certain diseases. This methodology is a way of silencing a specific gene, for example, genes that direct cancer cells to proliferate or that create overproduction of proteins that cause rheumatoid arthritis. Basically, RNAi works by scanning RNA templates that may cause a disease and cleaving that RNA template, and enzymes then destroying the template before it can complete its actions on the offending DNA. One of the key barriers to successful RNAi therapy is their finding their way to a specific site in the body and then the RNAi not degrading rapidly before it can do useful work. In one illustrative embodiment, RNAi, siRNA, RISC elements may be targeted by one or more plasmonic elements in order to seek out and destroy potentially harmful genetic elements and or other genetic processes.

As noted in the literature, clathrin heavy chain is known to be a cytosolic protein that functions as a vesicle transporter. However, the clathrin heavy chain exists not only in cytosol but also in cell nuclei. The p53 gene, in which mutations have been found in >50% of human cancers, encodes a protein that plays an important role in preventing tumorigenesis. clathrin heavy chain expression enhances p53-dependent transactivation, whereas the reduction of clathrin heavy chain expression by RNA interference (RNAi) attenuates its transcriptional activity. Moreover, clathrin heavy chain binds to the p53-responsive promoter in vivo and stabilizes p53-p300 interaction to promote p53-mediated transcription. Thus, nuclear clathrin heavy chain is required for the transactivation of p53 target genes and plays a distinct role from clathrin-mediated endocytosis (Enari, et al 2006). In one embodiment, p53 and or one or more other types of genes, their diseases and disorders, and or RNAi related activities may be efficaciously controlled and regulated, mitigated, prevented, and or modified via one or more plasmonic elements and one or more types of associated electromagnetic radiation, including any energy elements.

Referring again to FIGS. 1, 2, 3, 4, and 5, in another embodiment, but not limited to, one or more plasmonic elements and one or more types of associated electromagnetic radiation, including any energy elements efficaciously achieve therapeutic effect by deliberately controlling and regulating, or modifying faulty exocytosis and or endocytosis processes that produce disorders and diseases. This is a health critical situation, as the role of dopamine receptors and transporters; the excitability of dopaminergic neurons; and the regulation of extracellular dopamine levels in the brain, especially in relation to the diseased state, has proven to be imperative for a further understanding of dopaminergic neurotransmission as a whole. For example, dopaminergic neurotransmission critically depends on exocytotic release and neuronal uptake of dopamine, as well as on diffusion away from the release site. Once target cells are reached, dopamine can bind to and activate dopamine receptors. The subsequent cellular response depends on the type of dopamine receptor that is activated and the signal transduction mechanisms that are coupled to these receptors. Disturbances in one or more of the above-mentioned aspects of dopaminergic transmission could lead to severe neurological and neuropsychiatric disorders such as Parkinson's disease, depression, addiction, schizophrenia, attention deficit hyperactivity disorder, restless legs syndrome, Tourette syndrome, and the like, and in or more embodiments, one or more such disorders may be efficaciously treated via one or more plasmonic elements and one or more types of associated electromagnetic radiation, including any energy elements.

Referring again to FIGS. 1, 2, 3, 4, and 5, in another embodiment, but not limited to, during some activities one or more plasmonic elements and one or more types of associated electromagnetic radiation, including any energy elements beneficially interact with an externally applied magnetic field, like during NMR. Invention elements in one embodiment can also transport one or more regulatory approved NMR contrast agents as cargo for developmental imaging and diagnostic studies, thereby creating a novel hybrid embodiment that can yield new types or additional of diagnostic information. Since invention protein elements are electrically neutral, only minimal (e.g., no) structural distortion of the elements occurs in the presence of the magnetic field. Further, invention elements/contrast agent elements may also be capable of crossing cellular membranes, protects and thus extend the utility of the invention.

Referring again to FIGS. 1, 2, 3, 4, and 5, in another embodiment, but not limited to, one or more cargo elements may comprise, for example, one or more metal ions including, but not limited to, the gadolinium (III) chelate compounds of DTPA, DO3A, DOTA and other variations of these linear and macrocyclic ligands that act as targeted and or non-targeted contrast agents.

Direct Gd3+-OH2 chemical bonds, which exchange rapidly with other bulk H2O molecules, produce the mechanism whereby unpaired electrons on Gd3+ relax the proton nuclei of many nearby H2O molecules. Accordingly, the behavior of T1 contrast agents, such as those based on gadolinium requires good direct contact with tissue water molecules (spin-lattice relaxation mechanism) to be efficient. Thus, it is often preferable to bind them to the external surface of the carrier. (Hooker, et al. 2007) In one embodiment, one or more protein-based plasmonic elements facilitate better contact to tissue water because one or more contrast agents of one or more types are not located in the interior part of a protein-based plasmonic cage element (in its cavity), but rather, located on much more exposed non-complete and or partial plasmonic cage elements of one or more types. In one embodiment, one or more cage element 200 has one or more contrast agents of one or more types located on the outside part of cage element 200; or on both the inside and outside parts of element 200.

In another illustrative embodiment, one or more imaging or study elements comprise one or more treated manganese minerals, such as oxides, silicates, and carbonates for imaging and study enhancement.

Besides Gd3 complexes, there is another important class of contrast agents for MRI that is based on polysaccharide coated iron oxide particles. Their peculiarity stems from the fact that their blood half-life and distribution to different organs of the reticuloendothelial system (RES) depend upon the particle size (Aime, et al 1998). In one embodiment, one or more elements comprise one or more of a wide range of lanthano-invention labeled derivatives for custom-designed contrast agents.

In another embodiment, one or more plasmonic elements and one or more types of associated electromagnetic radiation, including any energy elements comprise one or more therapeutic agents in addition to one or more hybrid contrast and diagnostic agents.

In another illustrative embodiment, targeted and or non-targeted in vivo delivery of one or more plasmonic elements are internally and or externally monitored, directed, activated, deactivated and or regulated, locally and or at a remote distance by, for example, but not limited to, NMR, ESR, ultrasound, radio transmissions, and or biochemical reactions.

Additionally, in other embodiments, invention-forming hybrid NMR is combined with other techniques, such as ENDOR, which combines the best aspects of ESR and NMR, to yield high sensitivity and nuclear selectivity, respectively, for in vivo and in vitro studies.

In one embodiment, one or more different sized, paramagnetic coated, quantum dots, and or photonic dots are used as one or more contrast markers in magnetic resonance imaging (Mulder, et al., 2009) in conjunction with one or more invention elements. In other embodiments, one or more different sized quantum dots, and or photonic dots may be used in positron emission tomography (PET) for hybrid agent, in-vivo molecular imaging, or as fluorescent tracers in optical microscopy.

In another configuration, one or more types of one or more plasmonic elements and one or more types of associated electromagnetic radiation, including any energy elements comprise one or more radiodiagnostic agents for nuclear medicine.

Referring again to FIG. 2, in further illustrative embodiments, in addition to invention elements, other types of free-floating cargo that comprise a fluid, gas, or vapor; which free-floating cargo may be carried in a cavity forming element 200, 220, and or 206; for example, it may be one or more molecular ensembles for enabling a plasmonic element, and or agents for enhanced medical imaging, and or therapeutic agents.

Referring again to FIGS. 1, 2, 3, 4, and 5, in another embodiment, but not limited to, one or more types of invention platforms comprise one or more types of in vitro and or in vivo nanoscale, opto-electronic, electronic, and or quantum mechanical element, i.e., bio-molecular devices, which may be employed in a scalable, intelligent, biomolecular device platform. The platform may also be composed of one or more non-invention elements and devices, such as crystals, conductors, insulators, semiconductors, MEMS, and circuits, but not limited to such. And further, the platform may also be coated in one or more surfactants and or cosurfactants and or metals, elements, materials and substances.

Referring again to FIGS. 1, 2, 3, 4, and 5, in another embodiment, but not limited to, one or more types of invention platforms comprise one or more types of in vitro and or in vivo nanoscale opto-electronic, electronic, and or quantum mechanical elements and devices.

Referring again to FIGS. 1, 2, 3, 4, and 5, in another embodiment, but not limited to, one or more types of invention platforms comprise one or more types of in vitro and or in vivo nanoscale elements, components, devices, systems and or platforms in one or more configurations, which form connectors for carrying information from a storage, processing or communications element or device to another, of one or more types.

Referring again to FIGS. 1, 2, 3, 4, and 5, in another embodiment, but not limited to, one or more types of invention platforms comprise one or more types of in vitro and or in vivo information processing elements, components, devices, systems and or platforms such as, for example, but not limited to, encoders and decoders, memory, logic gates, registers, circuits, wiring and connectors, input and output elements, analog to digital and digital to analog converters and system architectures known in the art.

Referring again to FIGS. 1, 2, 3, 4, and 5, in another embodiment, but not limited to, one or more types of invention platforms comprise one or more types of in vitro and or in vivo nanoscale elements, components, devices, systems and or platforms that modify, process, manipulate, encode and decode, input, output, transmit, communicate, store and read various forms and types of information using a variety of suitable techniques known in the art, in vivo and in vitro.

A scalable information-processing invention platform may also include an encoder, e.g., a predetermined or specific electromagnetic, opto-electronic, photonic, and or quantum mechanical sequence that deliberately encodes at least a subset of the elements to take the form of specified sequence, as well as a decoder for reading information from at least a subset of the protein-based information processing elements.

Another example embodiment of encoders/decoders used with protein-based information processing elements is the use of NMR and ESR and other methods known in the art in conjunction with plasmonic elements that can effect and discern behaviors and physical characteristics at the nanoscale, in vivo and or in vitro. Another example of embodiment encoders/decoders is invention sourced photons of different wavelengths and photo detectors together with protein-based information processing elements.

Referring again to FIGS. 1, 2, 3, 4, and 5, in another embodiment, but not limited to, one or more types of invention platforms comprise one or more types of in vitro and or in vivo nanoscale information processing elements, components, devices, systems and or platform, which may follow and execute algorithms of one or more types expressed by biological processes and control laws, and or by geometrically derived algorithms such as graphs and Lie algebras, including Clifford algebras, and or by electromagnetic and or quantum mechanical effects, but not limited to.

Referring again to FIGS. 1, 2, 3, 4, and 5, in another embodiment, but not limited to, one or more types of invention platforms comprise one or more types of in vitro and or in vivo cognitive information processing element, device, and or platform of one or more types that follow and execute algorithms expressed by biological processes and control laws, by geometrically derived algorithms such as graphs and Lie algebras, including Clifford algebras, electromagnetic, and or by quantum mechanical effects, but not limited to.

Referring again to FIGS. 1, 2, 3, 4, and 5, in another embodiment, but not limited to, one or more types of invention platforms comprise one or more types of in vitro and or in vivo hybrid digital and analog information processing element, device, and or platform of one or more types, wherein enlisting the rich repertoire of biochemical reactions and adopting a nested hierarchical organization makes intermixing of digital and analog processing possible in bio-computing applications.

Referring again to FIGS. 1, 2, 3, 4, and 5, in another embodiment, but not limited to, one or more types of invention platforms comprise one or more types of in vitro and or in vivo nanoscale information processing elements, components, devices, systems and or platform that utilize one or more parts of the electromagnetic spectrum and or quantum mechanical effects generated by one or more invention elements as the basis of computation and or transmission and communication.

Referring again to FIGS. 1, 2, 3, 4, and 5, in another embodiment, but not limited to, one or more types of invention platforms comprise one or more types of in vitro and or in vivo nanoscale recording memory media or components, which may incorporate metals, ferromagnetic materials, and or ferroelectric materials and elements, and or may form into magnetic rings, and or may form vertically polarized magnetic domains and or form magnetic domains on isolated islands of one or more types.

Referring again to FIGS. 1, 2, 3, 4, and 5, in another embodiment, but not limited to, one or more types of invention platforms comprise one or more types of in vitro and or in vivo are nanoscale photovoltaic cells or components of one or more types.

Referring again to FIGS. 1, 2, 3, 4, and 5, in another embodiment, but not limited to, one or more types of invention platforms comprise one or more types of in vitro and or in vivo nanoscale batteries or components of one or more type for storing electronic charge.

Referring again to FIGS. 1, 2, 3, 4, and 5, in another embodiment, but not limited to, one or more types of invention platforms comprise one or more types of in vitro and or in vivo nanoscale environmental hazard-screening device, and or comprise an in situ remediation, removal and or sequestration component or system of one or more types.

Referring again to FIGS. 1, 2, 3, 4, and 5, in another embodiment, but not limited to, one or more types of invention platforms comprise one or more types of in vitro and or in vivo opto-electronic device, system or component of one or more types.

Referring again to FIGS. 1, 2, 3, 4, and 5, in another embodiment, but not limited to, one or more types of invention platforms comprise one or more types of in vitro and or in vivo nanoscale passive and or active linear or nonlinear optic components, and or particle detectors, and or other elements sufficient to implement in vivo or in vitro optical system arrays and methods.

Referring again to FIGS. 1, 2, 3, 4, and 5, in another embodiment, but not limited to, one or more types of invention platforms comprise one or more types of in vitro and or in vivo detection, diagnostic and tracking agents for chemical, biological, and or nuclear elements and activities, but not limited to such.

I Referring again to FIGS. 1, 2, 3, 4, and 5, in another embodiment, but not limited to, one or more types of invention platforms comprise one or more types of in vitro and or in vivo spin-based electronics element or system of one or more types.

Referring again to FIGS. 1, 2, 3, 4, and 5, in another embodiment, but not limited to, one or more types of invention platforms comprise one or more types of in vitro and or in vivo elements that exploit the Coulomb blockade-like properties of self-assembled proteins, wherein a single particle at a time may move through a transmembrane protein-based channel.

Referring again to FIGS. 1, 2, 3, 4, and 5, in another embodiment, but not limited to, one or more types of invention platforms comprise one or more types of in vitro and or in vivo elements that utilize and or exploit the Casimir effect, which is a small attractive force that acts between two closely parallel, uncharged conducting elements. It is due to quantum vacuum fluctuations of the electromagnetic field.

In some illustrative embodiments, one or more elements and or platforms and in one or more configurations are physically linked via molecular addends of one or more types, but are not limited to such addend types.

Referring again to FIGS. 1, 2, 3, 4, and 5, in another embodiment, but not limited to, one or more non-invention elements comprise one or more types of 3rd party therapy elements in whole or in part, such as one or more drug and pharmacological elements; biological elements; biomedical or medical elements; and the like, including healthcare elements; bioengineered elements; cosmetic elements; and the like. In one embodiment, one or more invention elements are adjunctive therapy elements to the 3rd party therapy elements.

Referring again to FIGS. 1, 2, 3, 4, and 5, but not limited to, in one embodiment, one or more 3rd party therapy cargo elements of one or more types comprise targeted and or non-targeted drug delivery elements, including their high precision dosing, or other forms of healthcare elements for diagnosing, remedying, inhibiting, mitigating, curing, and or preventing one or more types of diseases, infections, physical or mental trauma, or other forms of physical and mental afflictions. In one embodiment, one or more plasmonic elements and one or more types of associated electromagnetic radiation, including any energy elements are adjunctive therapy elements to 3rd party therapy elements.

Referring again to FIGS. 1, 2, 3, 4, and 5, but not limited to, in one embodiment, one or more plasmonic elements and one or more types of associated electromagnetic radiation, including any energy elements comprise an in vitro and or in vivo model and or system for research study, including a model, method, and or system for the research and development of new drugs, therapies, prosthetics, and drug delivery systems, including an accelerated drug discovery process. In one embodiment, one or more plasmonic elements and one or more types of associated electromagnetic radiation, including any energy elements are complementary R&D elements to 3rd party R&D elements.

Referring again to FIGS. 1, 2, 3, 4, and 5, in another embodiment, but not limited to, one or more plasmonic elements and one or more types of associated electromagnetic radiation, including any energy elements are utilized for studying, discovering, preventing, curing, mitigating, and or healing one or more types of animal, tree, plant, grain, grass, agricultural, vegetable, and or fungal diseases, disorders, infestations, and or blights.

Referring again to FIGS. 1, 2, 3, 4, and 5, in another embodiment, but not limited to, one or more plasmonic elements and one or more types of associated electromagnetic radiation, including any energy elements are used for studying, discovering, designing, and or enabling of genetically engineered elements, for example, one or more types of genes, cells, and other biological elements and products in animals, trees, plants, grains, grasses, agriculture, vegetables and fungi.

In another illustrative embodiment, one or more plasmonic elements and one or more types of associated electromagnetic radiation, including any energy elements comprise one or more methods for nourishing and or promoting healthy growth in one or more types of animals, trees, plants, grains, grasses, agriculture, vegetables and or fungi.

Referring again to FIGS. 2 and 4, in another embodiment, but not limited to, the heat shock cognate protein, hsc70, and its molecular co-chaperone auxilin, help to regulate the natural endocytosis aftermath of natural CCV uncoating and disassembly. Hsc70 also promotes uncoating and disassembly of Coatomer I and II vesicles. In cells over-expressing ATPase-deficient hsc70 mutants, uncoating of CCVs is inhibited in vivo. In one embodiment, bioengineered plasmonic elements may be used to regulate under or over expression of hsc70 and or auxilin. In one example embodiment, using a monoclonal antibody or other agent type as cargo against hsc70 blocks the hsc70-mediated release of invention and or non-invention clathrin from coated vesicles. In another example embodiment, or more auxilin elements comprise invention elements.

In one illustrative embodiment, one or more invention elements are stable with respect to dissociation, including one or more associated non-invention elements.

In another illustrative embodiment, disassembly and dissolution of one or more invention elements are deliberately inhibited and control and regulated, including one or more associated non-invention elements.

In one illustrative embodiment, one or more invention elements remain stable for a time certain or estimated time before the onset of dissociation, including one or more associated non-invention elements.

In one illustrative embodiment, dissociation of one or more invention elements may occur in whole or in part, including one or more associated non-invention elements.

In one illustrative embodiment, one or more elements may comprise one or more uncoating and dissociation agents and or use one or more methods for controlled and regulated release of agents or cargo from one or more elements, including one or more associated non-invention elements.

In another embodiment, disassembly and dissolution of one or more invention elements, including one or more associated non-invention elements are inhibited, controlled and regulated, and or promoted by using one or more specific agents, stimuli, and or other methods.

In one embodiment, but not limited to, one or more invention elements of one of more types are formed in vitro via the following protocols, which may be modified and or substituted by one or more other types of protocols in one or more invention embodiments: (Adapted from Campbell, C et al., Biochemistry 23, 4420-4426 (1984), Pearse & Robinson, EMBO J. 9:1951-7 (1984), and Zhu, et. al., Methods in Enzymology, 328, 2001, Kedersh N, et al., J. Cell Biology 103, 1986.) Adapted from Campbell, C et al., Biochemistry 23, 4420-4426 (1984), Pearse & Robinson, EMBO J. 9:1951-7 (1984), and Zhu, et. al., Methods in Enzymology, 328, 2001, Kedersh N, et al., J. Cell Biology 103, 1986.)

Part I. Method of Differential Centrifugation.
1. Make up 1 L of a buffer (buffer A) that comprises: 50 mM Mes pH 6.5, 100 mM NaCl, 1 mM EGTA, 0.5 mM MgCl2, 0.02% NaN₃, 1 mM DTT a day prior to experiment and storage at 4° C.
2. Add 1:100 PMSF proteases inhibitor to buffer A (200 ul/20 ml).
3. Collect and wash 14 rat brains (˜2.0 g) and livers (˜20.0 g). Wash and place the brains in ice-cold buffer A. Perfuse the livers with ice-cold PBS and collect them in ice-cold buffer A.
4. Mince and homogenize the brains in a Potter-Elvehjem grinder with 2 volume of ice-cold buffer A per total brain wet weight (˜90 ml). Do the same with the livers (˜400 ml).
5. Centrifuge the homogenate at 23,000 g (11,900 rpm) in a Sorvall GSA or at 13,000 rpm in a Sorvall SS34 rotor for 45 min at 4° C.
6. Collect the supernatant and centrifuge at 43,000 g (18,000 rpm) in a Sorvall SS34 rotor or at 20,000 rpm in a ti 45 Beckman rotor for 1 h at 4° C.
7. Resuspend the pellet in 10 ml of ice-cold buffer A, use a loose-fitting Teflon-glass Dounce homogenizer.
8. Collect homogenate in a 50 ml conical tube. Wash pestle and glass homogenizer with 5 ml of buffer A, and add this to homogenate until total volume is 15 ml. Add 1:100 PMSF
9. Dilute the homogenate 1:1 with 15 ml of 12.5% Ficoll/12.5% sucrose (both in ice-cold buffer A), and mix by inversion to ensure homogeneity.
10. Centrifuge at 43,000 g (18,000 rpm) in a Sorvall SS34 rotor or at 20,000 rpm in a ti 45 Beckman rotor for 30 min at 4° C.
11. Collect the supernatant in a graduate cylinder and dilute it 1:5 in ice-cold buffer A. Add 1:100 PMSF
12. Centrifuge the supernatant at 100,000 g (33,000 rpm) in a Beckman 70.1Ti rotor or at 31,100 rpm in a ti 45 Beckman rotor for 1 h at 4° C.
13. Collect pellet and resuspend in 5-10 ml of ice-cold buffer A by using a loose-fitting Teflon-glass Dounce homogenizer. Add 1:100 PMSF
14. Leave the homogenate on ice for about 30 min, and take an aliquot of 10 ul for EM, and dilute 1:10 for brain, 1:100 for liver.

Part II. Purification of CCVs Using Density Gradients (Zhu's CCVs and Clathrin Coat Preparation). Submit the Crude Clathrin-Coated Vesicles from Fresh Rat Brain to Discontinuous Sucrose Gradient for Remove Contaminating Vaults.
1. CCVs resuspended in (5-10 ml) buffer A
2. Preparer a discontinuous sucrose gradient in 5W28 tubes by carefully layering 5 ml of 40%, 5 ml of 30%, 6 ml of 20%, 8.5 ml of 10%, and 8.5 of 5% sucrose solutions in buffer A from bottom to top.
3. CCVs (5-10 ml) is laid on top of the gradient and centrifuged at 100,000 g (25,000 rpm) in a 5W28 rotor for 1 hr at 4° C.
4. Collect twenty-six 1.5 ml factions from the top.
5. Small aliquots from every other faction are analyzed for CCVs using 10% SDS-PAGE.
6. [Fractions comprising the CCVs (typically fractions 12-21 as numbered from the top of the gradient) are combined, diluted with 3 volumes of buffer A, and centrifuge at 112,000 g (31,100 rpm) in a ti 45 Beckman rotor for 1 h at 4° C. or at 33,000 rpm in a Beckman 70.1Ti rotor for 1 h at 4° C. Add 1:100 PMSF]
7. Resuspend the pellet in ice-cold buffer A, do a protein assay to yield an approximate concentration. Usually add 1 to 2 ml of buffer A.
8. Aliquot the homogenate in aliquots of 200 ul and store at −80° C. Take an aliquot of 10 ul each for EM and SDS-gel PAGE.

Part III. Isolation of Triskelia and APs from CCVs Using Keen's Method.
1. Dialyze CCVs against 0.01M Tris buffer, Ph 8.5, 3 mM azide for 5 hours.
2. Centrifuge at 240,000 g (51,200 rpm) for 20 min at 4° C. Because you are using low amount of sample; (IF we have less than 2 mL, Do not use the lid or close the centrifuge tubes of the 70.1 Ti rotor.) The soluble coat proteins comprising triskelial and APs are separated from the residual clathrin-coat vesicle membranes.
3. Collect the soluble fraction and do protein assay.
4. Take an aliquot of 10 ul for EM and 50 ul for SDS-gel PAGE.

Part IV. Separation by FPLC of AP-1 from AP-2 with Hydroxyapatite Column

Solutions:

| | | |
|---|---|---|
| Stocks: | 1M NaH₂PO₄; pH 7.1 | (30 g/250 ml) |
| | 5M NaCl | |
| | 10% NaN₃ | |
| Low PO₄ buffer | 10 mM NaH₂PO₄; pH 7.1 | (5 ml of stock) |
| (500 ml): | 100 mM NaCl | (10 ml of stock) |
| | 0.02% NaN₃ | (1 ml of stock) |
| | 0.1% beta-Mercaptoethanol | (0.5 ml) |
| | | (RT) |
| High PO₄ buffer | 500 mM NaH₂PO₄; pH 7.1 | (100 ml of stock) |
| | (200 ml):100 mM NaCl | (4 ml of stock) |
| | 0.02% NaN₃ | (0.4 ml of stock) |
| | 0.1% beta-Mercaptoethanol | (0.2 ml) |
| | | (RT) |

Both buffers need to be filtered and degassed prior to use.

| | | |
|---|---|---|
| AP buffer: | 100 mM MES, pH 7.0 | 39 g/2 l |
| | 150 mM NaCl | 17.5 g/2 l |
| | 1 mM EDTA | 4 ml of 500 mM solution/2 l |
| | 0.02% NaN₃ | 4 ml of 10% solution/2 l |
| | 0.5 mM DTT | -> add just before use (4° C.) |

Hydroxyapatite Column:

5 ml Econo-Pac CHT-II from BioRad; the column is stored at 4° C. in low PO₄ buffer

Procedure:
Connect the hydroxyapatite column to the FPLC system via the BioRad adaptors. Put a 0.2μ syringe filter at the inlet of the column.
Use the following FPLC settings:
   Sensitivity: 1
   Flow: 1 ml/min
   Chart Recorder speed: 0.5 cm/min
   Make sure the fraction collector is set at “ml” and a volume of “1”
Pump A is used for the low PO₄ buffer; Pump B for the high PO4 buffer. Wash the pumps with Valve 1 in position “3”.
Once the FPLC system is set up, start washing the column with 20 ml of high PO₄ buffer (=20 min). Be sure to switch on UV-Lamp.
This is followed by equilibration of the column with low PO₄ buffer; i.e. until the baseline is stable. The backpressure of the system should be approx. 0.1 MPa and must not exceed 0.35 Mpa.
During the equilibration phase (Valve 1 in position “1”=“Load”), the 50 ml superloop is loaded with the AP sample (Pump C; 5 ml/min).
With the column equilibrated and the superloop loaded, switch Valve 1 into position “2”=“Inject”. The APs are injected over the column at a flow rate of 1 ml/min.
After the injection is completed, continue running low PO₄ buffer over the column until the baseline is stable. Don't forget to prepare 1.5 ml tubes for the fraction collector.
AP-1 and AP-2 are then eluted from the column using Method 6:

| | | |
|---|---|---|
| 0.0 | CONC % B | 0.0 |
| 0.0 | VALVE.POS | 1.1 |
| 0.0 | CM/ML | 0.50 |
| 0.0 | PORT.SET | 6.1 |
| 40.0 | CONC % B | 0.0 |
| 40.0 | ML/MIN | 1.00 |
| 50.0 | CONC % B | 100 |

The elution profiles for AP-1 and AP-2 tend to vary considerably from one purification to another; AP-1 is eluted first.
AP-1 tends to be eluted from the column in three to four 1 ml fractions, usually starting at around #13. AP-2 is usually eluted in up to 15 fractions, starting at around #25. The fractions comprising the APs need to be verified by SDS-PAGE (two gels of 10% or 12%)
Wash column with low PO₄ buffer; store at 4° C.
Pooled AP-1 fractions and pooled AP-2 fractions are dialyzed against 1 liter of AP buffer overnight, and for a few more hours after exchanging the buffer (4° C.). The samples are then stored at 4° C.
Typically, the concentration for clathrin (peak fractions) is approx. 0.5 mg/ml, for AP-1 and AP-2 between 0.3-0.5 mg/ml.

Part V. Recombinant Clathrin Formation
According to one illustrative embodiment, but is not limited to, recombinant clathrin formation may be achieved in the following exemplar manner. Stoichiometric quantities of adaptor elements 208a (see FIG. 2, drawings) comprising AP-1 and AP-2 are required for clathrin self-assembly at physiological pH. However, in vitro clathrin self-assembly occurs spontaneously below about pH 6.5. Recombinant terminal and distal domain fragments are produced and combined with recombinant-produced hub fragments in assembly buffer as described below in order to induce formation of one or more clathrin elements, such as those comprising elements 206a, (see FIG. 2, drawings) for use in the invention.
In one illustrative technique, bovine clathrin heavy chain cDNA encoding heavy chain amino acids 1-1074 (SEQ ID NO: 1) is cloned into the pET23d vector (Novagen) between the NcoI (234) and XhoI (158) sites. Expression of the cloned sequence results in a terminal and distal domain fragments having a C-terminal polyhistidine tag. Hub fragments corresponding to amino acids 1074-1675 (SEQ ID NO: 2) are cloned into vector pET15b (Novagen) between the BamHI (319) and XhoI (324) sites. Expression of the hub fragments produces the proximal leg domain and central trimerization domain of the clathrin hub with an N-terminal polyhistidine tag. Vectors comprising the heavy chain and hub domains are expressed in E. coli by induction with 0.8 mM isopropyl-β-D-thiogalactopyranoside for 3 hours at 30 degrees Celsius. Expressed proteins are isolated, recombinant, and or synthetic from bacterial lysate in binding buffer (50 mM Tris-HCl (pH7.9), 0.5M NaCl, 5 mM imidazole) in a nickel affinity resin using the polyhistidine tag. Proteins are eluted with206a mM EDTA and dialyzed against 50 mM Tris-HCl (pH7.9). Hub fragments are further isolated, recombinant, and or synthetic using size exclusion chromatography on a Superose 6 column (Pharmacia).
In another exemplar technique, clathrin assembly reactions are performed using expressed heavy chain and hub fragments by overnight dialysis at 4 degrees Celsius in assembly buffer (100 mM 2-(N-morpholino) ethanesulfonic acid, pH 6.7, 0.5 mM MgCl2, 1 mM EGTA, 1 mM Tris (2-carboxyethyl)-phosphine hydrochloride, 3 mM CaCl2. Assembly reactions are centrifuged for 5 minutes at 12,000 rpm. The supernatant is then centrifuged for 45 minutes at 45,000 rpm (100,000×g). The pellets are resuspended in assembly buffer, and protein composition is determined on SDS-PAGE. The efficiency of element 206a formation can be determined by electron microscopy by diluting assembly reactions 1:5 in 10 mM Tris pH7.9, and placing aliquots on a glow-discharged carbon-coated grid, using 1% uranyl acetate as the stain.
According to another illustrative embodiment, but is not limited to, recombinant clathrin formation may be achieved in the following exemplar manner, as described by Rapoport, et al. (MBC 2008): A cDNA encoding rat clathrin heavy chain (Kirchhausen et al., 1987a) is used as a template to generate full-length (1675 HC), nested C-terminal truncations (1661 HC, 1643 HC, 1637 HC, 1630 HC, and 1596 HC), internal deletions (1675 PIVYGQ HC, 1643 PIVYGQ HC, and 1675 QLMLTA HC), and mutations (1643LML-AAA HC) of the heavy chain; each is then subcloned into the insect cell expression vector pFastBac1 (Invitrogen, Carlsbad, Calif.). A cDNA encoding rat liver clathrin light chain LCa (Kirchhausen et al., 1987b) is used as the template to subclone the region encoding the full light chain (residues 1-256) into the insect cell expression vector pFastBacHTb. The final construct (rLCali) comprises at its N terminus a 6x-His-tag followed by a linker of 20 residues. Baculoviruses suitable for infection and expression are generated with the Bac-to-Bac system (BD Biosciences, San Jose, Calif.). Virus stocks are obtained after four rounds of amplification, and they are kept in the dark at 4° C. The open reading frame of rat brain clathrin light chain LCa1 is also used as a template to subclone it into the bacterial expression vector pET28b (Novagen, Madison, Wis.) between the NcoI and EcoRI restriction sites so as to generate a native, nontagged light chain. All constructs are verified by DNA sequencing. clathrin heavy chains together with light chain are expressed in Hi5 insect cells (1 L, 1-1.5 206a cells/me grown for 2-3 d in spinner flasks at 27° C. in Excell 420 medium after coinfection with the appropriate viruses. Alternatively, clathrin heavy chain only is expressed in a similar way. The cells are centrifuged at 1000 rpm for 10 min at room temperature by using an H6000A rotor (Sorvall, Newton, Conn.), and the pellets are resuspended in 20 ml lysis buffer (50 mM Tris, pH 8.0, 300 mM NaCl, 1 mM EDTA, 3 mM mercaptoethanol, and half of a tablet of Complete Protease Inhibitor Cocktail [Roche Applied Science, Indianapolis, Ind.]). The resuspended pellets are sonicated for 1 min on ice (Flat tip at 20% power, Ultrasonic processor XL; Heat Systems, Farmingdale, N.Y.), cell debris is removed by centrifugation at 90,000 rpm for 20 min at 4° C. by using a TLA 100.4 rotor (Beckman Coulter, Fullerton, Calif.), and the supernatant (20 ml) is dialyzed at 4° C. for 12 h against 2×2 l of cage buffer (20 mM [2-(N-morpholino) ethanesulfonic acid] MES, pH 6.2, 2 mM CaCl2, 0.02% NaN3, and 0.5 m Mdithiothreitol [DTT]). The sample is then centrifuged at 4° C., first at low speed (1000 rpm for 10 min) to remove large aggregates and then at high speed (54,000 rpm for 1 h) by using a Ti rotor (Beckman Coulter). The pellet, primarily comprising clathrin (presumably assembled as cages) is resuspended in 6 ml of 100 mM MES, pH 6.5, 3 mM EDTA, 0.5 mM MgCl2, 0.02% NaN3, 0.5 mM DTT, and 0.5 mM phenylmethylsulfonyl fluoride) followed by addition of 3 ml of 2.4MTris, pH 7.4, 1 mM DTT, and incubation for 20 min at room temperature, a condition used to dissociate native clathrin assemblies. The sample is centrifuged at 90,000 rpm for 20 min at 4° C. by using a TLA 100.4 rotor, and most of the clathrin is recovered in the supernatant. The resulting sample is subjected to gel filtration chromatography (90 cm×0=3 cm column comprising Sephacryl-S 500 [GE Healthcare, Little Chalfont, Buckinghamshire, United Kingdom] in 0.5 M Tris, pH 7.4, 0.04% NaN3, and 0.5 mM DTT) at room temperature and with a flow of 2 ml/min. Fractions of 5.5 ml comprising the clathrin peak (100 ml) are pooled and then subjected to adsorption chromatography (5 ml, hydroxyapatite, Econo-Pac CHT-II; Bio-Rad, Hercules, Calif.); the column is pre-equilibrated with low phosphate buffer (10 mM NaH2PO4, pH 7.1, 100 mM NaCl, 0.02% NaN3, and 0.5 mM DTT) and eluted with a linear gradient from low to high phosphate concentration (500 mM NaH2PO4, pH 7.1, 100 mM NaCl, 0.02% NaN3, and 0.5 mM DTT) at room temperature with a flow of 1 ml/min. Fractions (1 ml) are collected into microcentrifuge tubes comprising 2 l of 0.5 M EDTA. Typical clathrin yields are in the range of 3-40 mg per 11 of cell culture. Western blot analysis is used to confirm the expression of clathrin heavy and light chains. The rat clathrin light chain rLCa1b is expressed in Escherichia coli strain BL21(DE3). The bacteria are grown in Luria-Bertani (LB) medium comprising 30 mg/l kanamycin at 37° C. with shaking (250 rpm) to an optical density of 0.5. Expression is induced by addition of isopropyl-d-thiogalactoside (IPTG) (final concentration, 0.6 mM). After 3 h, the cell are harvested by centrifugation at 5000 rpm for 10 min at 4° C. by using an H6000A rotor (Sorvall) and resuspended in ice-cold lysis buffer (20 mM Bis-Tris adjusted to pH 6.0 at room temperature, 0.5 mM dithiothreitol, 1 mM EDTA, and Complete Protease Inhibitor Cocktail) by using 20 ml of lysis buffer per 3.5 g of wet cell weight. The suspension is placed into a glass vessel, and the vessel is immersed in boiling water for 4 min and then chilled on ice. The boiled suspension is centrifuged at 54,000 rpm for 30 min at 4° C. by using a 60Ti rotor (Beckman Coulter) to remove the precipitated material. rLCa1b is purified from the filtered supernatant (0.2-m syringe filter) by anion exchange chromatography at 4° C. on a HiTrap MonoQ column equilibrated with buffer A (20 mM Bis-Tris, adjusted to pH 6.0 at room temperature, and 0.5 mM dithiothreitol) and eluted using a linear gradient from 0 to 32% buffer B (20 mM Bis-Tris, adjusted to pH 6.0 at room temperature, 0.5 mM dithiothreitol, and 1 M NaCl). For the in vitro reconstitution of clathrin, recombinant heavy chain (expressed in insect cells without light chain) is mixed with excess rLCa1b (expressed in bacteria) by using a weight ratio of 3:1 (equivalent to a molar ratio HC:LC of 1:2.4) just before cage or coat assembly for 40 min at room temperature.

Part VI. Clathrin Coat Formation

Reagents

Coat Formation Buffer

| | |
|---|---|
| 80 mM Mes hydrate pH 6.5 | 31.23 g/2 L |
| 20 mM NaCl | 2.34 g/2 L |
| 2 mM EDTA | 8 mL of 500 mM stock solution/2 L |
| 0.4 mM DTT | 1.6 mL of 500 mM stock solution/2 L |
| 2. clathrin | |
| 3. AP-2 | |

Procedure
(1) Place a solution of clathrin and AP-2 into a dialysis chamber
   clathrin: AP-2=3:1 to 4:1 (w/w)
(2) Dialyze over night against coat formation buffer; replace buffer and dialyze for an additional 3-4 h.
(3) Transfer to a centrifuge tube, centrifuge to remove larger aggregates
   rotor: TLA-100.4, 12000 rpm, 4° C., 10 min
(4) Transfer supernatant to fresh centrifuge tube, centrifuge to collect coats
   rotor: TLA-100.4, 65000 rpm, 4° C., 12 min
(5) Immediately withdraw supernatant with a 1 mL pipette.
(6) Wash carefully with buffer around the pellet.
(7) Resuspend the pellet by adding buffer, allowing to stand at room temperature for 10-15 min, then slowly wash buffer over the pellet to resuspend using a micro-pipettor (avoid foaming)
volume: 120-150 μL for a pellet of −3 mm diameter

Part VII. Clathrin Cage Formation

Reagents
1. Cage Formation Buffer:
20 mM Mes, pH 6.2 (3.9 g/l) (7.8 g/21)
2 mM CaCl2 (2 ml of 1M/l) (4 ml of 1M/2 l)
0.02% NaN3 (2 ml of 10%/l) (4 ml of 10%/21)
0.5 mM DTT (1 ml of 500 mM/l) (2 ml of 500 mM/21)

Clathrin

Procedure
(1) Place a solution of clathrin (0.5-1 mg/mL) into a dialysis chamber
(2) Dialyze over night against cage formation buffer; replace buffer and dialyze for an additional 3-4 h.
(3) Transfer to a centrifuge tube, centrifuge to remove larger aggregates
   rotor: TLA-100.4, 12000 rpm, 4° C., 10 min
(4) Transfer supernatant to fresh centrifuge tube, centrifuge to collect coats
   rotor: TLA-100.4, 65000 rpm, 4° C., 12 min
(5) Immediately withdraw supernatant with a 1 mL pipette.
(6) Wash carefully with buffer around the pellet.
(7) Resuspend the pellet by adding buffer, allowing to stand at room temperature for 10-15 min, then slowly wash buffer over the pellet to resuspend using a micropipettor
(avoid foaming)

Part VIII. Production of Recombinant Auxilin
A protein chimera of glutathione transferase (GST) with bovine auxilin (spanning residues 547-910) is generated by fusion in the vector pGEX4T-1 and then used for expression in E. coli BL21 (Fotin et al., 2004a). The bacteria are grown in LB medium supplemented with ampicillin to an OD600 0.5-0.6 at 37° C. Protein expression is induced by addition of 1 mM IPTG (final concentration) and the cells grown for another 4 h at 25° C. The cells (from 11 of culture) are centrifuged at 5000 rpm for 15 min at 4° C., and the pellet is kept frozen overnight. The pellet is resuspended in 25 ml of pGEX lysis buffer (20 mM HEPES, pH 7.6, 100 mM KCl, 0.2 mM EDTA, 20% glycerol, 1 mM DTT, and half a tablet of Complete Protease Inhibitor Cocktail) and sonicated on ice using three consecutive sonication cycles of 60, 30, and 30 s (standard microtip, 20% power). The sample is centrifuged at 45,000 rpm for 1 h at 4° C. by using a 60Ti rotor, and the supernatant mixed with 0.5 ml of a 50% (vol/vol) slurry of glutathione-Sepharose 4 beads (GE Healthcare). After 2 h of end-over-end rotation at 4° C., the beads are poured into a propylene Econo-Column (Bio-Rad), washed with 15 ml of pGEX lysis buffer, and then washed with 15 ml of 25 mM HEPES, pH 7.0, 100 mM NaCl, and 0.1 mM EGTA. Elution of GST-auxilin (in 2 ml) is achieved by supplementing the solution with 50 mM glutathione, adjusted to pH 8. These steps are carried out at 4° C. Release of the GST portion is achieved by incubation of 1 mg of GST-auxilin with 1 U of thrombin at room temperature for 6 h. Proteolysis is ended by addition of 1 mg of Pefabloc SC (Roche Applied Science). The 40-Da auxilin fragment is further purified using a Mono S column (Pharmacia, Peapack, N.J.). The sample is first dialyzed overnight against MES buffer A (50 mM MES, pH 6.7, 1 mM EDTA, and 3 mM-mercaptoethanol), and then it is loaded onto the column (pre-equilibrated with MES buffer A) and eluted with a linear gradient of buffer A and with MES buffer B (50 mM MES, pH 6.7, 500 mM NaCl, 1 mM EDTA, and 3 mM-mercaptoethanol) at a flow of 1 ml/min. The auxilin sample is stored at 80° C. with 20% glycerol (final concentration).

Part IX. Production of Recombinant Hsc70
N-terminal 6x-His-tagged bovine Hsc70 (full length) cloned into the pET21a vector is expressed in E. coli BL21. The bacteria are grown at 37° C. in LB supplemented with 0.1 mg/ml ampicillin to an OD600 of 0.5, transferred to 28° C., and induced with 0.1 mM IPTG for 5 h. The cells are centrifuged at 5000 rpm for 15 min at 4° C., and the pellets from 11 culture resuspended in 25 ml 50 mM Tris, pH 8.0, 300 mM NaCl, 1 mM ATP, 2 mM MgCl2, 10 mM-mercaptoethanol, and half a tablet of Complete Protease Inhibitor Cocktail without EDTA. The supernatant obtained after sonication and centrifugation (as with auxilin) is mixed with 1 ml of 50% (vol/vol) slurry of nickelnitrilotriacetic acid-agarose beads (QIAGEN, Valencia, Calif.) for 4 h by endover-end rotation at 4° C. The beads are placed into an Econo Pac column and then washed with 30 ml of 50 mM Tris, pH 8.0, 300 mM NaCl, 10 mM-mercaptoethanol, 10 mM imidazole, 1 mM ATP, and 1 mM MgCl2). Hsc70 is then eluted at 4° C. with 5-6 ml of the same solution supplemented with 200 mM imidazole. Fractions of 1 ml are collected into microcentrifuge tubes comprising 40 l of 0.1 M EGTA. The samples comprising 20% glycerol (final concentration) are stored at 80° C.

Part X. Coatomer Formation
According to one illustrative embodiment, the coat protein I (COPI) assembly process is carried out by preparing Coatomer subunits from cytosolic preparations, including methods, but are not limited to, as essentially described in Spang, et al., Proc. Natl. Acad. Sci. USA. 1998 Sep. 15; 95 (19): 11199-11204. Coatomer, a nanoscale element composed of seven distinct subunits (alpha, beta, beta′, gamma, delta, epsilon and zeta subunits, respectively) and ADP-ribosylation factor (ARF, an N-myristylated small GTP-binding protein) are the only cytoplasmic proteins needed.
In another illustrative embodiment, the coat protein I (COPI) assembly process is carried out by preparing Coatomer subunits from cytosolic preparations, including methods, but are not limited to, as essentially described in Sheff, et al, The Journal Of Biological Chemistry, Vol. 271, No. 12, Issue Of March 22, Pp. 7230-7236, 1996 “Purification of Rat Liver Coatomer (COPI”)—Purification of rat liver Coatomer is accomplished through a substantial modification of the method of Waters and Rothman (13). Unless otherwise noted, all operations are performed at 4° C. Approximately 250 g of fresh liver from 10-15 adult Sprague-Dawley rats (Harlan Sprague-Dawley) are homogenized in 2 volumes of buffer (25 mM Tris, pH 7.5, 320 mM sucrose, 500 mM KCl, 2 mM EDTA, 1 mM dithiothreitol) comprising protease inhibitors (2 mg/ml pepstatin A, antipain, and leupeptin; 1 mM phenylmethylsulfonyl fluoride) using a polytron homogenizer with 1.5-cm cutter assembly at maximum speed for three 1-min bursts on ice with 1-min rests. The lysate is cleared by sequential centrifugation at 9000 3 g for 15 min followed by centrifugation of the supernatant at 100,000 3 g for 1 h. This material (S100) is stored at 270° C. for up to 4 months. For a typical purification, 150 ml of S100 is diluted 6-fold with cytosol buffer (25 mM Tris, pH 7.5, 1 mM dithiothreitol, 1 mM EDTA plus protease inhibitors as above). Protein concentration is 5 mg/ml. Ammonium sulfate is added to 25% of saturation and stirred for 15 min on ice, and then precipitate is removed by centrifugation, and the supernatant is brought to ammonium sulfate at 45% of saturation with stirring on ice. The precipitate is collected by centrifugation and redissolved in 150 ml of cytosol buffer. An additional 120 ml of cytosol buffer is added and then 30 ml of 60% (w/v) polyethylene glycol 3350 in distilled H2O with gentle stirring. The mixture is incubated at 4° C. for 30 min, and the precipitate is collected by centrifugation at 10,000 3 g for 15 min. The precipitate is resuspended in 20 ml of G buffer (10 mM Tris, pH 7.5, 0.2 mM ATP, 0.2 mM CaCl2), the insoluble material is removed by centrifugation, and the supernatant is passed over a 20-ml column comprising 250 mg of DNase-I (Sigma) coupled to agarose (Affi-Gel-10, Bio-Rad, prepared according to the manufacturer's directions) to remove contaminating actin and actin binding proteins. Eluent is desalted into cytosol buffer using 10DG desalting columns (Bio-Rad) and applied to a 50-ml DEAE cellulose column (DE52, Whatman) equilibrated in cytosol buffer. COPI is eluted with a 100-400 mM KCl gradient over 200 ml, with the elution of COPI followed by spot blot on nitrocellulose using EAGE antibody. In a final step, peak COPI fractions are pooled, diluted 1:1 with cytosol buffer, and applied to a 1-ml Mono-Q column (Pharmacia) equilibrated in cytosol buffer and mounted on a fast protein liquid chromatography apparatus (Pharmacia). The column is swished with 300 mM NaCl and then eluted with a 350-400 mM NaCl gradient over 20 ml. COPI, as assayed by the presence of b-COP on a spot blot using EAGE antibody, eluted as a single peak. The presence and purity of COPI is confirmed by SDS-PAGE. An alternative final step is employed in preparing samples for two-dimensional dimensional gels. Here, DEAE eluent is concentrated in a Centricon-30 microconcentration (Amicon) to 400 ml and applied to a 24-ml Superose-6 (Pharmacia) column equilibrated in cytosol buffer with 50 mM KCl. As with Mono-Q, COPI eluted in a single peak. This final step produces a somewhat lower yield and comprises some contaminants between 30 and 100 KD by SDS-PAGE. For copurification of labeled CHO cytosol and rat liver COPI, all quantities are divided by 3, 1 ml of labeled cytosol is added to 50 ml of rat liver S100, and the Mono-Q column is used as the final step.

According to another illustrative embodiment, clathrin and or Coatomer I/II proteins are extracted and prepared from clathrin and or Coatomer I/II coated vesicles obtained from non-rat, non-bovine organic tissue, including from human tissue, in whole or in part. In another embodiment, clathrin and or Coatomer I/II coated proteins are extracted and prepared from clathrin and or Coatomer I/II coated vesicles obtained by donor/recipient tissue matching using established techniques. In another embodiment, clathrin and or Coatomer I/II proteins are prepared, in whole or in part, by using stem cells, cloning and or other genetic manipulation techniques known in the prior art to produce genetically matched tissue for a donor recipient.

The increasing interest in the targeting of foreign moieties at sites in the body where their activity is required is addressed by the invention in one or more embodiments. It is important that agents, like drugs, particularly those having undesirable side effects, are delivered to the site where they are supposed to act. Many molecular species require that they be delivered in a site specific manner, often to particular cells, for example, polynucleotides (anti-sense or ribozymes), metabolic co-factors or imaging agents. One such system has been described by Wu et al., J. Biol. Chem., 263, 14621-14624 and WO-A-9206180, in which a nucleic acid useful for gene therapy is conjugated with polylysine linked to galactose which is recognized by the asialoglycoprotein cargo attachment elements on the surface of cells to be targeted. However, there are many occasions, such as in the delivery of a cytotoxic drug, when it would not be satisfactory to use a delivery system in which the targeting and or masking moiety and or vector to be delivered is so exposed. This need is addressed by various delivery system embodiments of the invention that possess the flexibility to target a wide range of biologically active foreign moieties.

In one embodiment, the invention includes one or more elements having one or more suitable sites for subsequent attachment of a targeting and or masking moiety and or vector, and one or more elements having one or more surfaces and or protein coats to which one or more targeting and or masking moieties and or vectors have already been attached.

In one embodiment, one or more masking moieties are attached to the surface of one or more invention elements. These masking moieties prevent the recognition by a specific cell surface and instead allows for intravenous administration applications. For example, the surface masking characteristics may be provided by poly (ethylene glycol) (PEG) by using various PEG-PLA and PLGA mixtures. PEG conjugation masks the protein's surface, reduces its renal filtration, prevents the approach of antibodies or antigen processing cells and reduces its degradation by proteolytic enzymes. In one embodiment, PEGylated elements significantly improve element stability and prevent leakage of agents from elements. Studies have shown that protein-based nanoparticles and liposomes without PEGs have a short circulation time due to rapid uptake by macrophages of the reticulo-endothelial system (RES), primarily in the liver and spleen. Finally, PEG conveys to molecules its physico-chemical properties and therefore modifies biodistribution and solubility of peptide and non-peptide nanoparticles. Thus, recent studies have used mostly nanoparticles with PEGs. The PEG coating is highly hydrated and this layer protects against interactions with molecular and biological components in the blood stream, as well as nonspecific binding to tissue. In one embodiment, one or more elements, in one or more configurations, are internally and or externally attached, coated, and treated, in whole or in part by using steric stabilizers including, but not limited to, steric stabilizers selected among dipalmitoyl phosphatidyl ethanolamine-PEG, PEG-stearate, the esters of the fatty acids from the myristic acid to the docosanoic acid with methyl ether PEG, the diacylphosphatidyl ethanolamines esterified with methyl ether PEG and the polylactates and the polyglycolactates esterified with methyl ether PEG. In one embodiment, one or more elements are not required to be PEGylated to efficaciously operate.

In another embodiment, one or more elements, and in one or more configurations are internally and or externally coated or treated in whole or in part with surfactants, including, but not limited to, surfactant agents selected among soy-bean phosphatidylcholine, dioleyl phosphatidylcholine, dipalmitoyl phosphatidylcholine, hydrogenated soy-bean phosphatidylcholine, phosphatidylethanolamine and phosphatidylserine), and or with cosurfactants, including, but not limited to cosurfactant agents selected among ethanol, propanol, isopropanol, butanol, sodium taurocholate, sodium glycocholate, propylene glycol, butyric acid and benzoic acid.

In one or more embodiments, ligands can be of one or more efficacious types, such as drugs, and may be bioengineered, and or comprise isolated, recombinant, synthetic, and or cloned elements.

In one embodiment, one or more types of ligands may be functionalized and or attached in one or more ways to one or more elements.

In one embodiment, ligands are natural ligands of one or more types. In another embodiment, one or more types of natural ligands are modified and or functionalized. In another embodiment, invention element ligands and natural element ligands are combined to comprise one or more types of hybrid ligand elements.

In another embodiment, the course of a natural ligand and or invention ligand element during cellular signaling, trafficking, downregulation, upregulation, endocytosis, exocytosis, and other cellular entry or exit, cellular inter- and or intra-actions, and the like, may be efficaciously controlled, regulated, and or modified by one or more plasmonic elements and one or more types of associated electromagnetic radiation, including any energy elements to yield one or more diagnosis, cure, mitigation, treatment, prevention of disease, or other types of efficacious effects, and the like.

Examples of some natural ligands, but not limited to, are listed below that may be subject to efficacious control, modification, and or regulation in one or more invention embodiments:

Toxins and lectins, e.g.,

### Diptheria Toxin

Pseudomonas toxin
Cholera toxin

### Ricin

### Concanavalin A

### Viruses, e.g.,

Rous sarcoma virus
Semliki forest virus
Vesicular stomatitis virus

### Adenovirus

### Influenza

### West Nile

Serum transport proteins and antibodies, e.g.,

### Transferrin

Low density lipoprotein

### Transcobalamin

Yolk proteins

### IgE

Polymeric Ig
Maternal I g
IgG, via Fc receptors
Hormones and Growth Factors, e.g.,
Insulin
Epidermal Growth Factor
Growth Hormone
Thyroid stimulating hormone
Nerve Growth Factor
Calcitonin
Glucagon
Prolactin
Luteinizing Hormone
Thyroid hormone
Platelet Derived Growth Factor
Interferon
Catecholamines
LDL
Neurotransmitters
Substance P
A neurotransmitter known to stimulate pain receptors

In one or more embodiments, one or more invention elements are conjugated (bonded) with one or more other elements (e.g., ligands), agents, materials, and or substances of one or more types, including those developed by 3^{rd} parties, which may be used singly or mixed together in one or more element configurations for medical and biological research, diagnosis, therapy, or prosthetic purposes. One or more biomedical elements such as ligands and other types of biomedical functionalization elements may be directly and or indirectly attached, bonded, fastened, cross-linked, and or affixed to and or incorporated into one or more invention elements, as well as one or more non-invention and or natural elements. In one embodiment, attachment is achieved via molecular tethers. In another embodiment, no molecular tether is involved. In one configuration, a free radical molecule may be attached directly to one or more invention elements. In another embodiment, one or more elements may be bonded, fastened, and or affixed to one or more elements by being included in a modified protein sequence of one or more elements or bonded elements; by using a spacer; by covalent bonding; by site directed mutagenesis; by genetically engineered mutation and or modification; by peptides; by proteins; by DNA; by antibodies; by monoclonal antibodies; by recombinant elements; and via other bioengineering techniques and methods known in the art.

According to one embodiment, the protein amino acid sequence of one or more elements are modified to provide a site suitable for attachment thereto of an in vivo or in vitro targeting and or masking moiety. In one illustrative embodiment, one or more target-specific ligands and or targeting moieties are directly attached to one or more elements via one or more amino acid groups, and or attached via one or more short molecular tethers.

In another embodiment, one or more functionalization elements, of one or more types, comprise highly specific targeting agents, such as, but not limited to, antibodies, peptides or small molecules, large molecules, and other functional ligands, such as fluorophores and permeation enhancers, and the so functionalized nanoparticles may target receptors, transporter, enzymes and or intracellular processes in vivo with high affinity and specificity.

In one illustrative embodiment, one or more elements, such as diagnostic, therapeutic, prosthetic, and or assay agent elements, but not limited to, are delivered to a target in vivo or in vitro using a variety of guidance techniques, including for example, optical (photonic), acoustic, electric, biological, chemical, mechanical reactions and forces, but not limited to, and one or more elements may be delivered singly and or in one or more configurations to one or more targets.

In another illustrative embodiment, one or more plasmonic elements and one or more types of associated electromagnetic radiation, including any energy elements comprise one or more diagnostic agents and or effects to perform site designation, site specificity, and site retention for targeted in vivo delivery of therapeutics; the latter may also comprise part of the same diagnostic payload.

In one illustrative embodiment, the invention enables targeted agent delivery systems that retain their structural integrity and that may also loiter for a calculated period of time at the targeted area of concern after delivery of agent payload.

In one illustrative embodiment, one or more elements comprise molecules arranged in specific patterns. The pattern of elements precisely mirrors or mimics a spatial or physical pattern a target cell in a human or animal body expects to see and will recognize, and one or more elements are accepted by the target cell, which can be a cancer cell or HIV infected cell, for example.

In one embodiment, one or more nanoparticle plasmonic elements comprised of one or more types of metals, e.g., noble, alkali, and or other suitable metals, with sensor ligands and using electrical charges are bonded to one or more elements, attached to ligands, targeting moieties, and or to vectors, and used together with one or more invention elements. The metal particles carry short strands of artificial DNA (oligonucleotides) tailored to match known segments of biological DNA that are implicated in, or linked to, disease.

Target-specific ligand binding and any subsequent changes within or to one or more plasmonic elements and one or more types of associated electromagnetic radiation, including any energy elements may be a result of either covalent or non-covalent interactions—the latter including hydrogen bonding, ionic interactions, Van der Waals interactions, and hydrophobic bonds—depending on the application, system design, receptor design, cargo type and or the interaction/application environment.

In another illustrative embodiment, one or more elements, ligands, targeting moieties, vectors, and the like utilize the method of chirality.

In another illustrative embodiment, reactions and forces arise from one or more ligands and or targeting moieties binding to targets, including covalent and non-covalent interactions, which ligands are tethered and or directly attached to one or more invention elements. Ligand binding to one or more specific targets may produce one or more conformational changes sufficient to deform and or rupture one or one or more elements in whole or in part, thereby causing one or more elements to be released and effects to occur. The targeting moieties can be selected by one of ordinary skill in the art keeping in mind the specific cell surface to be targeted. For example, if one wishes to target the asialoglycoprotein receptor on the hepatocytes in the liver, an appropriate targeting moiety would be clustered trigalactosamine. Once a specific targeting moiety has been selected for a particular cell to target, the different targeting moieties can be attached either by covalent linkage directly onto the surface of one or more invention elements, or by indirect linkage via, for example, a biotin-avidin bridge. In another embodiment, depolymerization (e.g., by cytosolic Hsc 70) of the clathrin and or Coatomer element exposes one or more transmembrane proteins (V-SNARE) that direct one or more elements to their destinations by binding to a specific T-SNARE protein on the target organelle. The fusion protein SNAP25 causes the one or more elements to fuse with the target membrane

In one embodiment, avidin is attached covalently to the surface of one or more elements and a biotinylated ligand attaches non-covalently to the avidin. In another embodiment, biotin is covalently attached to the surface of one or more invention elements, and then avidin is used as a bridge between the biotinylated polymer and the biotinylated ligand. Targeting agents may also include one or more biocompounds, or portions thereof, that interact specifically with individual cells, small groups of cells, or large categories of cells. Examples of useful targeting agents include, but are not limited to, low-density lipoproteins (LDS's), transferrin, asiaglycoproteins, gp120 envelope protein of the human immunodeficiency virus (HIV), and diphtheria toxin, antibodies, and carbohydrates. A variety of agents that direct compositions to particular cells are known in the prior art (see, for example, Cotten et al., Methods Enzym, 1993, 217, 618).

In another illustrative embodiment, one or more classical structural activity relationships (SARs) based drug discovery approaches are combined with one or more other techniques to form a specific case of targeted drug delivery, for example, but not limited to, one or more structural metabolism relationships (SMRs) that in combination with SARs are sometimes termed as retrometabolic drug design approaches. These active drugs are designed to undergo singular metabolic deactivation after they achieve their therapeutic roles, and may produce specific action at the site of application without affecting the rest of the body.

In another illustrative embodiment, one or more plasmonic elements and one or more types of associated electromagnetic radiation, including any energy elements comprise one or more functionalities and or methods that produce targeting by changing molecular properties of an overall target molecule, as a result of enzymatic conversion, but also, for example, may involve one or more pharmacophores. These elements, sometimes referred to as the targetor (Tor) moiety, are converted by site-specific enzymes to active functions. In addition to the Tor moiety, one or more other functions may be introduced into elements for in vivo use, which can be named as “protector functions” that serve as lipophilicity modifiers or protectors of certain functional groups in therapeutic agent molecules.

In other illustrative embodiments, one or more other types of targeting delivery systems and methods can be used, for example, but not limited to, in whole or in part in one or more configurations and combinations: electromagnetic, photoelectric, thermal, quantum mechanical, and or other invention elements, states, and or effects, including lasing; surfactants (surface-active substances) and or cosurfactants; enzymatic physical-chemical-based targeting; site-specific enzyme-activated targeting; vectors, such as ligand-based, non-viral-based, and Protein/DNA polyplex vector targeting; receptor-based chemical targeting; organic and or inorganic synthetic elements; transmembrane proteins (V-SNARE); peptides, including peptides that cross cell membranes and home specifically to certain diseases; nanostructured dendrimers and hyperbranched polymers; molecular Trojan horses; adenovirus, herpes simplex virus, adeno-associated virus or other virus vectors for targeted delivery that do not cause toxicity; antibodies, including monoclonal antibodies; nanoparticles, including polymer nanoparticles like polymer, polybutylcyanoacrylate, and ethyl alcohol nanoparticles; immunotoxins; hormonal therapy; tissue-specific gene expression; gene therapy; pegylated immunoliposomes; anti-sense therapy; biological elements and or agents, including biological elements and agents conjugated with other agents, such as transferrin, but not limited to such; chemical elements and agents; devices, systems, and or mechanisms; liposomes, including liposomes conjugated with transferrin, but not limited to such; conformationally-constrained peptide drugs targeted at the blood-brain barrier; endogenous blood brain barrier and or blood tumor capillary transporters; inhibiting and or modulating blood brain barrier active efflux transporters; air and or other gas bubbles; blood brain barrier breaking and or disrupting elements and agents; blood brain barrier tight junction separating and or endocytoses elements and agents; vector-mediated delivery of opioid peptides to the brain; brain drug delivery of peptides and protein drugs via vector-mediated transport at the blood brain barrier; neurotrophic elements; neuroprotective elements; and or various peptides, and drugs, and the like.

In another illustrative embodiment, one or more plasmonic elements cross various in vivo biological barriers, such as the transmucosal passage, and may also cross the blood-brain barrier (BBB) and the blood-cerebrospinal fluid (CSF) barrier for targeted and or non-targeted in vivo delivery of CNS agents and elements. In one embodiment, one or more BBB-passing therapy elements also comprise small and or large molecule drugs.

Natural clathrin, and in particular its ability to ‘track’ vesicle proteins leaving a synapse into the extracellular space (Granseth, et al 2007) indicates that the protein is not immediately scavenged by phages and other “housecleaning” elements in the brain, and further, may move freely about CNS spaces. In one embodiment, one or more invention elements efficaciously move through the CNS spaces and comprise in situ elements, which plasmonic elements and one or more aspects of electromagnetic radiation perform remediation, removal, and or sequestration of one or more types of contaminants, toxic elements, undesirable organic or inorganic elements, and the like.

In another embodiment, extensive modification and functionalization of elements may not be required for CNS entrance and or BBB passage. Only minimal functionalization may be required, depending on element type.

In another embodiment, one or more CNS-entering and or BBB-passing plasmonic elements and one or more types of associated electromagnetic radiation, including any energy elements may behave as a drug by themselves—i.e., they efficaciously operate alone without carrying additional cargo elements, e.g., therapeutic cargo elements. In another embodiment, one or more plasmonic elements carry one or more adjunctive therapy elements of one or more types past the BBB.

In another illustrative embodiment, one or more invention elements enter the CNS and or cross the blood brain barrier for targeted delivery of elements, including, but not limited to, one or more plasmonic elements and one or more types of associated electromagnetic radiation, including any energy elements, energies, states, and or effects, small and or large molecules, non-lipid-soluble micromolecules, macromolecules, light sources, hydrophilic and or hydrophobic agents, such as therapeutic, diagnostic, and prosthetic agents, and other structured cargo to specific cells and areas within the brain, and such agents and or cargo may comprise one or more sensor agents, assay agents, diagnostic agents, prosthetic agents, and also may comprise agents like central nervous system drugs, antibiotics, and antineoplastic agents of one or more types, but are not limited to such.

In another embodiment, one or more elements are capable of circumventing the fluid-brain barriers by intracellular routes related to three separate and distinct endocytic processes. The three endocytic processes from the least to the most specific are fluid- or bulk-phase endocytosis, adsorptive endocytosis, and receptor-mediated endocytosis.

There are several transport mechanisms and techniques known in the art to be involved in the uptake of nanoparticles by the brain across the BBB (Lockman et al. 2002, Begley, 2004, de Boer et al. 2007), one or more of which may be utilized in one or more invention embodiments. These mechanisms and techniques include: simple diffusion of lipophilic molecules, the BBB-specific influx transporters, including organic anion and cation transporters and transcytosis or endocytosis. In one embodiment, one or more elements are internalized at the BBB by one or two different endocytosis mechanisms: receptor-mediated endocytosis (RME) and adsorptive-mediated endocytosis (AME). AME is triggered by an electrostatic interaction between the positively charged moiety of the peptide and the negatively charged region of the plasma membrane. In contrast, RME is specific to certain peptides such as insulin and transferrin.

In one embodiment, delivery through the blood-brain barrier of one or more types of plasmonic elements is accomplished via chimeric peptides. The latter are formed when a transportable vector, such as cationized albumin, lectins, or a receptor-specific monoclonal antibody, is conjugated to a therapeutic compound that is normally not transported through the BBB. In one embodiment, conjugation of drugs to transport vectors is facilitated by, but not limited to, the use of avidin-biotin technology. In another embodiment, chimeric peptides are not required to pass through the blood-brain barrier, depending on cargo and element types.

In another illustrative embodiment, one or more elements may be coated with one or more surfactants and or cosurfactants, including, but not limited to, polysorbate 20, 40, 60 and 80, and or with one or more other materials and substances to cross various biological barriers, such as the transmucosal passage, and also to overcome the blood-brain barrier (BBB), the transmucosal passage, and the blood-cerebrospinal fluid barrier (CSG) for targeted delivery of agents and elements nanoparticles. In another embodiment, surfactants and or cosurfactants are not required to achieve such BBB-passing functionality, depending on cargo and element type. E.g., in the prior art, it has been shown that using such surfactants and co-surfactants can cause an immunogenic response.

In another illustrative embodiment, one or more elements may be cationized to facilitate blood brain barrier passage. In another embodiment, cationization is not required to achieve such functionality, depending on cargo and element type.

In another illustrative embodiment, one or more elements cross the blood brain barrier due to disruption of the barrier by acoustic techniques, such as by using ultrasound.

In another embodiment, zonula occludens toxin and its eukaryotic analogue, zonulin, (zot) are protein ligands attached to one or more invention elements. Zonulin, the natural ligand of the Zot target receptor, interacts with these cargo attachment elements at the blood brain barrier, unlocking the tight junctions (TJ) in the brain that regulate the blood-brain barrier at that receptor. TJ-unlocking allows passage of one or more elements through the BBB, and thereby enables delivery of small and large molecules, non-lipid-soluble micromolecules, macromolecules, light sources, and other structured cargo elements to the brain. In another embodiment, Zonulin is not required to pass through the blood-brain barrier, depending on cargo and element types.

Extracellular pathways circumventing the fluid-brain barriers in humans are comparable in the CNS of rodents and a subhuman primate. The most highly documented extracellular route is through the circumventricular organs (e.g., median eminence, organum vasculosum of the lamina terminalis, subfornical organ, and area postrema), all of which comprise fenestrated capillaries and, therefore, lie outside the BBB. In one embodiment, blood-borne macromolecules—specifically fluid-phase molecules released by the invention—escaping fenestrated vessels supplying the circumventricular organs move extracellularly into adjacent brain areas located behind the BBB.

The potential intracellular and extracellular pathways that blood-borne substances carried within one or more elements may follow in various embodiments for circumventing the fluid-brain barriers and entry to the CNS are therefore numerous, and various invention embodiments are used as appropriate. One invention embodiment, for example, uses the nasal cavity as a route for delivery of one or more types of elements, effects, and or other agents, especially for systemically acting drugs that are difficult to deliver via routes other than injection. Embodiments for the use of the nasal cavity for drug delivery also extend to circumventing the blood brain barrier. Drugs have been shown to reach the CNS from the nasal cavity by a direct transport across the olfactory region situated at the loft of the nasal cavity. It is the only site in the human body where the nervous system is in direct contact with the surrounding environment. In one embodiment, the nasal route would be important for rapid uptake of one or more types of drugs used in crisis treatments and management, such as for acute pain, epilepsy, psychic agitation, and for one or more other types of centrally acting drugs where the pathway from nose to brain provides a faster and more specific therapeutic effect. Furthermore, in another embodiment, the trigeminal nerve and, in animals, the vomeronasal organ also connects the nasal cavity with the brain tissue. One or more methods of nasal delivery to the CNS, which may also be used by the instant invention, but not limited to, are described in Dhuria, et al, 2008; Ma et al, 2007; and Thorne et al. 1995.

The nasal cavity has a relatively large absorptive surface area and the high vascularity of the nasal mucosa ensures that absorbed compounds are rapidly removed (Mainardes, et al 2006). In one embodiment, two routes, singly or in combination, are used via which one or more types of molecules are transported from the olfactory epithelium into the CNS and/or CSF. The first is the epithelial pathway, where one or more types of compounds pass paracellularly across the olfactory epithelium into the perineural spaces, crossing the cribriform plate and entering the subarachnoid space filled with CSF. From here the molecules can diffuse into the brain tissue or will be cleared by the CSF flow into the lymphatic vessels and subsequently into the systemic circulation. The second embodiment utilizes the olfactory nerve pathway, where compounds may be internalized into the olfactory neurones and pass inside the neuron through the cribriform plate into the olfactory bulb. In another embodiment, it is possible that further transport into the brain can occur by bridging the synapses between the neurons. After reaching the brain tissue, the drugs are cleared either via the CSF flow or via efflux pumps such as p-glycoprotein at the BBB into the systemic circulation. Despite the potential of the nasal route, there are some factors that limit the intranasal absorption of drugs. These barriers include the physical removal from the site of deposition in the nasal cavity by the mucociliary clearance mechanisms, enzymatic degradation in the mucus layer and nasal epithelium and the low permeability of the nasal epithelium removed (Mainardes, et al 2006). Colloidal carriers systems, such as nanoparticles and liposomes have demonstrated great efficacy in increasing drug bioavailability via the nasal route (Illum, 2002) In one invention embodiment, one or more elements comprise a colloidal carrier for enhanced nasal delivery of one or more elements, of one or more types.

Further, in one embodiment, it is possible to greatly improve the nasal absorption of one or more types of adjuntive drugs and plasmonic elements and one or more types of associated electromagnetic radiation, including any energy elements by administering them in combination with an absorption enhancer that promotes the transport of the drug across the nasal membrane. Another invention embodiment comprises a nasal drug-delivery system that combines an absorption enhancing activity with a bioadhesive effect, which increases the residence time of the formulation in the nasal cavity. In one embodiment, this method can be even more effective for improving the nasal absorption of polar drugs. In one or more embodiments, a wide range of absorption enhancer systems can be utilized. In another embodiment, depending on cargo and element types, minimal functionalization may be required to take advantage of nasal absorption for efficacious passage to brain cells.

In another illustrative embodiment, one or more plasmonic elements and one or more types of associated electromagnetic radiation, including any energy elements, and in one or more configurations comprise in vivo and or in vitro sensor systems, assay systems, therapeutic drugs and other suitable methods to do genetic-based (trait-based) and or phenotype (state-based) drug dosing. In one embodiment, drugs are delivered at optimally effective and safe doses per each individual.

The invention, in one embodiment, provides for individual patient factors such as genotype, phenotype, age, gender, ethnicity etc., to be taken into account by one or more plasmonic elements and factored into dosing and administration consideration. It has been demonstrated that inter-individual response variability can be 40-fold or more with practically all classes of psychotropic drugs. This makes it difficult to formulate rational guidelines for dosing and interpretation of biological parameters (such as plasma or serum drug concentrations) that might be associated with a therapeutic response. Although much remains unknown, a number of factors have been characterized as important determinants of patient-to-patient variability. These encompass genetics, disease state, nutritional status, concurrent use of drugs, and other pharmacoactive substances, including demographic factors such as age, gender, and ethnicity. Therefore, there is a requirement for in vivo systems that analyze many of these factors and dynamically adjust dosing accordingly.

In one embodiment, one or more plasmonic elements and one or more types of associated electromagnetic radiation, including any energy elements comprise one or more personalized medicine elements, and which elements' and or effects' efficacy may be increased, because responses arising from one or more individual variability factors; such as, but not limited to, genotype, phenotype, disease state, metabolic state, nutritional status, coinstant use of drugs, and other pharmacoactive substances, and also demographic factors such as age, and ethnicity; are factored into the elements, pre-delivery and or post delivery. Side effect profiles may also be reduced via such personalized medicine embodiments.

In one embodiment, one or more plasmonic elements also comprise one or more adjunctive patented drugs; drugs that are about to go off patent; have already gone off patent (generics); and or their active metabolites, and which drugs' efficacy may be beneficially altered and or enhanced by use of the invention. These beneficial changes in the status of an existing drug may be achieved by utilizing the invention in one or more plasmonic elements and one or more types of associated electromagnetic radiation, including any energy aspects embodiments and configurations, for example, but not limited to: target specific areas in the body; to pass the blood brain barrier; to cross over into cells and their organelles; to fuse with cell membranes; to gain access to the cytosol; to offer the benefits of low antigenicity or minimal immunogenic effects; to modify, regulate, and or control cellular processes; to more efficiently and efficaciously carry drugs; and or to dynamically and or statically adjust the drug's responses and dosages arising from inter-individual variability due to one or more factors, such as, but not limited to, genotype, phenotype, disease state, metabolic state, nutritional status, coinstant use of drugs, and other pharmacoactive substances, and also demographic factors such as age, gender, and ethnicity of the patient. New patent filings for about to go off patent drugs and drugs already off patent may be enabled by one or more plasmonic elements, such as affording increased drug efficacy, and or by enabling a better safety profile for the drug in question.

In various embodiments, the instant invention can also carry one or more types of adjunctive biomedical or healthcare elements, for example and without limitation: one or more electromagnetic, photoelectric, thermal, quantum mechanical and or other invention elements; quantum medicine elements; therapeutic elements; pharmaceutical elements; diagnostic elements; assay elements; cosmetic elements; agents for treating one or more types of autoimmune diseases; agents for treating one or more types of infectious diseases; biological elements; radioactive agents or nuclear medicine agents; contrast agents; nano-scale biosensors; restorative agents; regenerative agents; cell, tissue, organ or circulatory repair elements; drug discovery agents; drug designer agents; drug research and development agents; drug fabrication agents; drug control and regulation agents; drug modifier agents; targeted drug delivery agents; clinical drug trial agents; antibiotics; antibacterials; vaccines; antiviral and anti-parasitic drugs; cytostatics; vitamins; proteins and peptides, including enzymes; hormones or other biological elements; prosthetic elements; intelligent nano-prostheses that supplement or enhance cell, tissue, or organ functioning; surgical elements; magnetic iron oxide nanoparticles; nano-scale biosensors; assays; diagnostic systems or nano-devices for in vivo delivery of targeted therapy to combat diseases, such as cancer and HIV, and the like, including other types and forms of drug elements for the diagnosis, cure, mitigation, treatment, prevention of disease. Some or all such elements may operate under the control and influence of one or more plasmonic elements and one or more types of associated electromagnetic radiation, including any energy elements, and comprise another type of invention platform.

In another illustrative embodiment, one or more plasmonic elements and one or more types of associated electromagnetic radiation, including any energy elements in whole or in part, and cure, mitigate, or treat one or more types of bodily injuries and insults, including traumatic injury, blood clots, and the like, but not limited to.

In one embodiment, nano-engineered scaffolds are composed of a plurality of plasmonic elements, which are able to support and promote cellular differentiation and growth in injured or degenerated regions.

In one illustrative embodiment, one or more plasmonic elements and one or more types of associated electromagnetic radiation, including any energy elements also comprise one or more types of adjunctive small and or large molecules and may utilize one or more methods to enter the CNS and or cross the blood brain barrier, in whole or in part, for delivery of one or more assay, diagnostic, therapeutic agents, and drugs, of one or more types to cells and or targeted areas within the brain, like, for example: one or more invention states, effects, and or other elements; contrast agents; central nervous system drugs; antibiotics; antineoplastic agents, which may be used for treating malignant brain tumors (primary and or metastasized, of one or more types) or benign neoplasms; Parkinson's agents; Multiple Sclerosis agents; epilepsy agents; meningitis agents; Alzheimer's disease agents; HIV infection agents; memory agents; stroke agents; coma agents; and the like; or comprise one or more psychotropic agents or therapies of one or more types to study, diagnose, cure, mitigate, or treat of one or more types of mental health and illness, including, but not limited to, stress; anxiety; depression; mania; bipolar disorder; attention deficit (hyperactivity) disorder; panic attacks; phobias; addictions; anger; rage; suicidal thoughts and tendencies; substance abuse disorder; post traumatic stress disorder; psychoses; mental retardation; autism; delirium symptoms; schizophrenia; neuroses; and or enhancing memory; cognition; cognitive functioning; the effects of cognitive therapy, and the like; including other types and forms of drug elements for the diagnosis, cure, mitigation, treatment, or prevention of one or more types of CNS diseases.

In another illustrative embodiment, one or more plasmonic elements enter the CNS, including crossing the blood brain barrier, in whole or in part, to diagnose, cure, mitigate, or treat one or more types of CNS injuries and insults, including traumatic brain injury, blood clots, and the like, but not limited to. In one embodiment, such CNS elements are used for beneficial effect.

In one embodiment, one or more plasmonic elements and one or more types of associated electromagnetic radiation, including any energy elements promote neuroprotection by limiting the damaging effects of free radicals generated after head injury, a major factor contributing to neuropsychiatric degenerative disorders (e.g., Alzheimer's).

In one embodiment, nano-engineered scaffolds composed of a plurality of plasmonic elements are able to support and promote neuronal differentiation and growth in injured or degenerated brain regions.

Compounds such as drugs, amino acids, carbohydrates, proteins, nucleotide bases, hormones, pesticides and co-enzymes have been successfully used in the prior art for the preparation of selective recognition matrices. A wide variety of print molecules have been used in various imprinting protocols known in the art. Of all the imprinting strategies known in the art, it has become evident that the use of non-covalent interactions between the print molecule and the functional monomers is the more versatile. The apparent weakness of these interaction types, when considered individually, may be overcome by allowing a multitude of interaction points simultaneously. Together with the fast association and dissociation kinetics of these bond types, so that in a short time many possible combinations can be checked before the correct partners associate, this protocol has proven advantageous. Furthermore, the use of non-covalent interactions in the imprinting step closely resembles the recognition pattern observed in nature. Example invention molecular imprint embodiments in the art include, but are not limited to:
Fragmented polymer monoliths
Composite polymer beads
Polymer beads from suspension, emulsion or dispersion polymerization
In-situ polymerization
Polymer particles bound in thin layers
Polymer membranes
Surface-imprinted polymer phases

In one illustrative embodiment, the invention uses molecular-imprint technology, wherein biodegradable films are used as a pliable template for elements, which elements are pressed into a film and then removed, leaving a physical mold of the element's shape. In one embodiment, this can facilitate catalysis of certain reactions and may also be used for shape selective separations. In other embodiments, imprinted polymers may facilitate the fabrication of elements to achieve selective diffusion; as chromatographic supports for the separation of enantiomers and oligonucleotides by invention elements; to provide the recognition element for an invention chemical sensor; and for the synthesis of polymeric materials that mimic biological cargo attachment elements and are targeted by invention elements, and or play a role in the design of new drugs. In one embodiment, this invention process provides for imprinted biodegradable capsule production with target or site-specific feature sizes at the molecular level. Other invention embodiments may utilize imprinted membranes and thin films that also function as an artificial cell wall for the selective transport of targeted drugs, peptides and biologically important molecules.

Surface imprinting involves the following steps: The print molecule, usually a large one, is first allowed to form adducts with functional monomers in solution and the formed elements are subsequently allowed to bind to an activated surface such as silica wafers or glass surfaces. Thus, with this technique, a designed imprinted (imaged) surface is obtained. This approach should potentially be valuable for creating specific cell binding surfaces. When preparing molecularly imprinted polymer monoliths against large imprint species, there is a risk of permanent entrapment of the template in the polymer after polymerization. When using thin polymeric layers or imprinted surfaces this drawback may be overcome.

In one embodiment, imprinted nanocapsules using techniques known in the art and as discussed above, one or more elements utilize and or constitute a nanocapsule with manifold, multi-tiered capabilities for in vivo administration and targeted delivery. The imprinted nanocapsule is delivered in vivo to detect and target a particular in vitro imprinted biological element, which may be, but is not limited to, a particular type of receptor, protein, or cell, since its imprint shape on the nanocapsule will only bind in vivo to that particular biological element target. The molecular-level imprint process thereby provides for targeting one or more elements using biodegradable nanocapsules for in vivo agent delivery. In addition, vectors and targeting moieties, and blood brain barrier, transmucosal, and CSF barrier breaching elements, and other elements and substances may also be attached to the surface of the molecular imprint nanocapsule or otherwise be conjugated to it.

In another illustrative embodiment, one or more elements may be used in conjunction with molecularly imprinted polymers known in the art as recognition elements in biosensor-like devices. In one embodiment, imprinted polymer embodiments may be highly resistant sensing element alternatives.

In another illustrative embodiment, one or more elements are encapsulated in whole or in part in one or more biodegradable controlled-release polymers, which polymers may also be conjugated with other elements and agents. The polymer capsule, and or one or more elements may also be coated with one or more surfactants and or cosurfactants and or with other materials and substances. One or more targeting and or masking moieties and or other targeting vectors may also be attached on the polymer surface, and or on one or more elements.

In one embodiment, one or more elements are put into one or more biodegradable controlled-release polymeric capsules, and these elements transform “dumb” polymeric delivery capsules into “smart” systems.

In the instance of polymeric nanocapsules, which may be molecular imprinted or not, illustrative controlled-release polymeric nanocapsule embodiments of the invention may include one or more of the following delivery systems, but not limited to, and in one or more configurations:

Diffusion-controlled systems

Water penetration-controlled delivery devices

Chemically controlled systems

Drugs covalently attached to polymer backbone systems, which delivery systems can be further subdivided into soluble systems and insoluble systems. Insoluble systems are used as a subcutaneous or intramuscular implant for the controlled release of the chemically tethered therapeutic agent. Soluble systems are used in targeting applications.

Drug release determined predominantly by erosion systems, whereby certain polymers can undergo a hydrolysis reaction at decreasing rates from the surface of a device inward, and under special circumstances the reaction can be largely confined to the outer layers of a solid device. Two such polymers are poly (ortho esters) and polyanhydrides, because the rates of hydrolysis of these polymers can be varied within very wide limits, considerable control over the rate of drug release can be achieved.

Poly (ortho esters) systems, which are highly hydrophobic polymers that comprise acid-sensitive linkages in the polymer backbone.

Polyanhydrides materials as bioerodible matrices for the controlled release of therapeutic agents. Aliphatic polyanhydrides hydrolyze very rapidly while aromatic polyanhydrides hydrolyze very slowly, and excellent control and regulate over the hydrolysis rate can be achieved by using copolymers of aliphatic and aromatic polyanhydrides. In this way, erosion rates over many days have been demonstrated, and erosions rates measured in years have been projected.

The form in which the foreign moiety, vector and or cargo are held within one or more plasmonic elements and one or more types of associated electromagnetic radiation, including any energy elements will depend on the release properties and methods required. For release at the targeted site, it will be important to ensure that the right conditions prevail, for example, to permit cell localization and internalization via receptor mediated endocytosis.

In one illustrative embodiment, the invention enables one or more types of delivery systems whose plasmonic elements engage in an iterative, interactive, and dynamic dialog with one or more targets; follow a sequence of actions using: one or more invention states, effects and or elements; and or effects governed by biological control laws and methods; and or use behaviors and methods as defined by graphs and or an algebra, for example, a Lie algebra. In one illustrative example, one or more plasmonic elements and one or more types of associated electromagnetic radiation, including any energy elements follow an algorithm expressed by the invention, such as in this illustrative embodiment:

One or more elements, that may be with or without cargo elements, docks and or loiters on or near one or more cell membranes,

One or more elements enter one or more target cells, while one or more other elements continue to loiter nearby or stay docked at the cell membrane.

The docked and or loitering element elements wait for a time period,

The targeted cell produces one or more reactions, for example, manufactures and or secretes an agent in response to the element's docking and or delivering its cargo,

The docked element and or loitering elements analyze the new cell behavior and or its secretions,

The docked element or loitering elements undergo a conformational change in response to the cell's new behavior,

The docked element and or loitering elements self-adapt, producing yet another conformational change in the cell, and or releases another round of one or more agents that are taken up by the targeted cell, and,

The foregoing process is repeated as required to achieve an efficacious effect.

In another embodiment, one or more plasmonic elements and one or more types of associated electromagnetic radiation, including any energy elements operate in an intelligently staged sequence or orchestrated series of actions, which may be multiplexed or done in parallel.

By using one or more one or more plasmonic elements operate on one or more elements that are sensitive to one or more types of effects, which may also comprise one or more invention element-entrapped agents. This method results in a staged series of overall actions that follow an intelligently ordered sequence of events. In an example embodiment, first a diagnostic agent entrapped by one or more plasmonic elements is released by an electromagnetic, photoelectric, thermal, quantum mechanical, and or other type of trigger, and the agent's positive finding of a disease, like cancer or HIV then causes one or more therapeutic agents to be released from the same and or other one or more other elements by one or more triggers. Agent dosages are released in calculated amounts, and the dosages may be non-targeted or targeted.
In another illustrative embodiment, cavity-forming elements, which may be cage, vesicle, shell, and or cargo elements, have one or more compartments that in whole or in part are separated by one or more barriers, for example, but not limited to, one or more phospholipid membrane barriers and or one or more barriers composed of molecular-imprinted films. The barriers may exhibit structural transitions due to internal or external stimuli. In one embodiment, agents or cargo entrapped within one or more elements remain sequestered within their respective compartments until a change in barrier permeability state is triggered by contact, for example, by a ligand, with one or more specific targets or sites. The subsequent biochemical and or biological reactions cause the barriers to alter states into an opened state and release entrapped cargo and agents from one or more invention elements. In one example embodiment, binary mixtures of therapeutic and or diagnostic agents are mixed together as needed to dynamically and more efficaciously deal with a disease or disorder.
The invention, in one or more embodiments, comprises in whole or in part one or more plasmonic elements and one or more types of associated electromagnetic radiation, including any energy elements, components, devices, systems, and the like, of one or more types, formed by using one or more engineering disciplines and related engineering technology disciplines of one or more types. Listed below are some such example invention embodiments, but are not limited to.
In one embodiment, the invention remedies the deficiencies of prior art by providing one or more plasmonic elements, a plurality of which may also comprise one or more nanoscale platforms of one or more types. A platform according to the invention may be used, for example, in biomedical, electronics, telecommunications, and information processing applications.

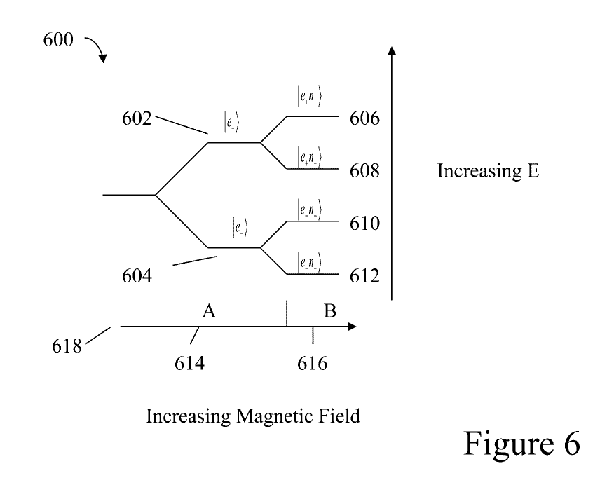

FIG. 6 is an exemplary energy level diagram 600 illustrating the energy levels associated with a hyperfine interaction between electron and nuclear spin in the presence of magnetic fields of the type used to do ESR spin label studies, which may be done in vivo and in vitro in one invention embodiment. The hyperfine interaction is a strictly quantum mechanical phenomenon. In an atom, the electron possesses an intrinsic quantum mechanical quantity known as spin. The nucleus of an atom also possesses spin. Intrinsic spin tends to generate a spin magnetic moment that is capable of interacting with other magnetic moments and fields. Generally, the spin magnetic moment of the nucleus does not interact with the spin magnetic moment of the electron. However, in the presence of a strong magnetic field, the spin magnetic moments of the electron and nucleus become coupled and interact.

In one illustrative embodiment, the electron is excited using pulses of electromagnetic radiation while maintaining its spin configuration. The source of the electromagnetic radiation may be, for example, an invention element, an ordinary lamp, an LED, a time-varying magnetic field generator, a laser, or an electromagnetic field generator. A hyperfine interaction gives rise to electron nuclear double resonance (ENDOR) techniques. According to one illustrative embodiment of the invention, room temperature EPR and ENDOR techniques known in the art are used for performing in vivo spin probe studies.

In another embodiment, one or more one or more plasmonic elements use quantum information processing techniques known in the art can modify, process, manipulate, encode and decode, input, output, transmit, communicate, store and read information using one or more modulated signals, methodologies, or carrier signals of one or more types.

In one embodiment, one or more one or more plasmonic elements and one or more types of associated electromagnetic radiation, including any energy elements in one or more configurations, are bonded, tethered, or otherwise incorporated into one or more invention and or non-invention elements, comprising functionalized nanoscale elements, components, devices, systems, and or platforms such as, but not limited to, nano-lasers, quantum dots; photonic dots; nanoscale DNA chips; protein assay chips; assay elements; environmental, protein, phenotype, DNA, and or metabolic assay and analysis elements.

In another embodiment, one or more one or more plasmonic elements may comprise a bio-lasing-spasing structure, in vivo or in vitro.

In one embodiment, one or more one or more plasmonic, electromagnetic, thermal, photoelectric, and or other invention elements in one or more configurations comprise nano-sensor elements; including, but not limited to, radioactivity sensors; chemical sensors; biological sensors; electromagnetic sensors; acoustic sensors; visible, infrared, and or ultraviolet wavelength sensors; tactile sensors; pressure sensors; volumetric sensors; flow sensors; and temperature sensors; and one or more of which sensors may constitute a bio-molecular device.

In one embodiment, one or more plasmonic elements and or platforms utilize and or employ one or more types of transmitter and or receiver elements as sensors and or for transmission of information of one or more types in vivo and in vitro.

In another embodiment, one or more plasmonic elements and one or more types of associated electromagnetic radiation, including any energy elements and in one or more configurations comprise one or more nanoscale electromagnetic, thermal, photoelectric, and or other invention elements, components, devices, systems, and or platforms that input, read out, process, analyze, output and report on information gathered by one or more types of diagnostic, test, label, tag, reporter, sensor, and or assay elements.

In one embodiment, one or more elements are released in vivo or in vitro from one or more elements, and the one elements are coated in whole or in part in one or more surfactants, cosurfactants, and other materials or sequestering substances.

In one embodiment, one or more one or more plasmonic elements are tagged to one or more other elements. A specific emitted wavelength of an invention element enables the identification of specific pathologies, disorders, metabolic states, proteins or DNA making it possible to diagnose various diseases.

In one embodiment, one or more nanoscale plasmonic elements and one or more types of associated electromagnetic radiation, including any energy elements comprise assay elements, which, using tiny permutations of color, can tag a million or more different proteins or genetic sequences in a process called multiplexing. In one embodiment, one or more plasmonic elements are excited at the same wavelength but have different emission wavelengths, and act as probes in experiments where multiple fluorescent measurements need to be made simultaneously, such as flow cytometry or confocal microscopy.

In another illustrative embodiment, one or more plasmonic elements and one or more types of associated electromagnetic radiation, including any energy elements are sufficient to implement, in vivo and or in vitro, one or more types of genetic and protein nanoscale optical biological assay systems and methods. In one illustrative configuration, one or more plasmonic elements comprise one or more nano-scale DNA chips, and or one or more nano-scale DNA chips to detect DNA samples formed from bonding with the target DNA on a chip, and or reference DNA nano-chips.

In another illustrative configuration, one or more one or more plasmonic elements and one or more types of associated electromagnetic radiation, including any energy elements comprise one or more protein array techniques. The array surfaces are designed to bind to one or more hydrophobic, hydrophilic (cation or anion) or specific ligands, and may also include a protein array reader.

In another illustrative embodiment, one or more plasmonic elements are used in a multiplexed analysis system or method that provides a nanoscale replacement for DNA-chip technology and can be used for the analysis of genetic variance, proteomics, and gene expression.

In another embodiment, one or more plasmonic elements and one or more types of associated electromagnetic radiation, including any energy elements produce one or more states and or effects caused by their coming into contact with a particular metabolic state, medical disorder, disease pathology, genotype, phenotype and or other specific stimuli.

In one embodiment, one or more transported agents carried by one or more plasmonic elements are selectively triggered and released. In doing so, they form a targeted agent delivery system without exposing the entire body—or an indiscriminate area—to a similar dose. The agents may be delivered in vivo by means known in the art and or as described herein.

In one illustrative embodiment, one or more plasmonic elements and one or more types of associated electromagnetic radiation, including any energy elements elements operate on one or more other elements that may have one or more entrapped materials, such as, but not limited to, therapeutic, diagnostic, and or therapeutic agents within an aqueous interior, and or that may have one or more entrapped nanoparticles such as liposomes, micelles, proteins, other biological and or bioengineered elements, including organic, inorganic, and synthetic materials, and or that may have one or more hydrophobic materials bound to a lipid bilayer membrane.

In one embodiment, the well-known permeability increase at the phase transition temperature provides a means to trigger release of an entrapped agent, like, for example release of a therapeutic agent in locally heated tissues. In one embodiment, efficient in vivo or in vitro release of entrapped agents at non-targeted and or targeted sites are triggered by light emitted by one or more plasmonic elements when the one or more elements also comprise a photoisomerisable species.

In another embodiment, the method of one or more LuxR proteins and lux bioluminescence genes and or other luminescent causing genes known in the art are utilized and are bioengineered and incorporated into one or more plasmonic elements and one or more types of associated electromagnetic radiation, including any energy elements, ligands, targeting moieties, and or vectors, which may also be conjugated with one or more other elements, materials, and substances. In one embodiment, luminescent causing genes provide optical pumping sufficient to excite one or more plasmonic elements.

In one embodiment, ultrabright, mammalian derived lux gene elements may be used that are in safe for both in vitro and vivo use. The bacterial luciferase (lux) gene cassette consists of five genes (luxCDABE) whose protein products synergistically generate bioluminescent light signals exclusive of supplementary substrate additions or exogenous manipulations. Historically expressible only in prokaryotes, the lux operon has been re-synthesized through a process of multi-bicistronic, codon-optimization to demonstrate self-directed bioluminescence emission in a mammalian HEK293 cell line in vitro and in vivo. An example described in the prior art, U.S. Pat. Nos. 7,371,538, 7,300,792, 7,208,286, 7,090,992, 6,905,834, 6,673,596, and 6,544,729 describe autonomous in vitro light production that was shown to be 12-fold greater than the observable background associated with untransfected control cells. The availability of reduced riboflavin phosphate (FMNH2) was identified as the limiting bioluminescence substrate in the mammalian cell environment even after the addition of a constitutively expressed flavin reductase gene (frp) from Vibrio harveyi. FMNH2 supplementation led to a 151-fold increase in bioluminescence in cells expressing mammalian codon-optimized luxCDE and frp genes. When injected subcutaneously into nude mice, in vivo optical imaging permitted near instantaneous light detection that persisted independently for the 60 min length of the assay with negligible background. This prior art shows that the speed, longevity, and self-sufficiency of lux expression in the mammalian cellular environment can provide a viable and powerful alternative for real-time target visualization not currently offered by existing bioluminescent and fluorescent imaging technologies.

In another embodiment, one or more LuxR proteins and lux bioluminescence genes and or other luminescent causing elements known in the art are utilized and are bioengineered and incorporated into one or more invention elements, their bonded elements, ligands, targeting moieties, and or vectors, which may also be conjugated with one or more other elements, materials, and substances.

Bioluminescence is turned on by the presence of certain diseases, disorders, metabolic states, pathogens, toxins, genotypes and phenotypes, as well as chemical, nuclear, and biological elements and activities, but not limited to such.

Bioluminescence light-emitting reactions are quite distinct for different organisms, with the only common component being molecular oxygen. Therefore, significant differences have been found between the structures of the luciferases and the corresponding genes from one luminescent organism to another. These distinctive properties may be manipulated via bioengineering techniques known the art to produce distinctive bioluminescence BSE embodiments.

According to one illustrative embodiment, luxA and luxB genes encode the a and b subunits of luciferase (a heterodimer in the form ab). The lux CDE genes code for polypeptides (transferase, synthetase, and reductase) that are required for the conversion of fatty acids into the long-chain aldehyde required for the luminescent reaction. The aldehyde is formed when an acyl group that was transferred from the synthetase to the reductase is reduced with NADPH. Beforehand, the fatty acid was activated by synthetase to form acyl-AMP, which in turn reacts with a specific cysteinyl residue located close to its carboxyl terminal. Acylation of the synthetase, in contrast to acyl-AMP formation, occurs efficiently only when the synthetase is bound to the reductase subunit. The fatty acid activated by the synthetase was formed when the transferase accepted the fatty acyl group onto a serine residue from acyl-ACP, acyl-CoA, and other acyl donors followed by the transfer to water or other acceptors. The cleavage, activation, and reduction of fatty acyl groups to form fatty aldehyde are highly specific for substrates with chain lengths of 14 carbons, providing strong evidence that tetradecanal is the natural substrate for the bioluminescent reaction.

According to one illustrative embodiment, one or more parts of the electromagnetic spectrum and or quantum mechanical effects generated by one or more plasmonic elements comprise one or more nano-computer elements, components, devices, systems and or platforms of one or more types that are programmable, and or autonomous acting, and or do cognitive processing, which bio-nano-computers may also utilize self-replicating, self-adapting, self-repairing, self-regulating, and or self-regenerating methods, and which are used for applications at the cellular, molecular, and nanoscale level that may include, but are not limited to, biomedical imaging, sensors, diagnostic systems, assay systems, therapeutic systems, drug delivery systems, prosthetic systems, cybernetic systems, cellular-level nano-fabrication systems, and inter- and intra-cellular imaging, repair, and engineering systems, the monitoring, sensing, imaging, diagnosing, repairing, constructing, fabricating, and or control and regulating of organic and or inorganic elements, and which bio-nano-computer elements and or platforms also may utilize and leverage biological control and regulate laws and or methods, and or geometrically derived algorithms such as graphs and Lie algebras, including Clifford algebras, but not limited to, in the performance of their tasks.

In one illustrative embodiment, one or more element chains are created via a molecular bridge group to align the elements with respect to one another and also with respect to an external magnetic or electrical field. In one embodiment, one or more plasmonic elements and one or more types of associated electromagnetic radiation, including any energy elements and or platforms and in one or more configurations are embedded in another material, like liquid crystal.

In one embodiment, one or more plasmonic elements and or platforms and in one or more configurations are coated completely and or partially in a metal.

In another embodiment, one or more plasmonic elements and one or more types of associated electromagnetic radiation, including any energy elements and or platforms and in one or more configurations are coated completely and or partially in reflective and or non-reflective coatings.

In one embodiment, one or more plasmonic elements and or platforms and in one or more configurations are used to coat completely and or partially metals, crystals, insulators, conductors, semiconductor components, wires, and devices.

In another illustrative embodiment, one or more plasmonic elements and one or more types of associated electromagnetic radiation, including any energy elements and or platforms and in one or more configurations facilitate the externally and or mechanistically directed alignment of one or more types of elements, for example, but not limited to, biological elements, various other non-invention nanoparticles, carbon nanotubes, crystals, conductors, semiconductors, insulators, and or other devices, materials and substances; which aligned assemblies may further be coated in one or more surfactants and or metals, elements, materials and substances.

In one embodiment, one or more elements in one or more configurations include one or more other types of nanoparticle elements such as, but not limited to, polymer-based, polybutylcyanoacrylate-based, and cetyl alcohol-based nanoparticles, empty cage Fullerenes, endohedral Fullerenes, carbon nanotubes, cells, liposomes, capsids, dendrimers, micelles, and the like.

In another illustrative embodiment, one or more plasmonic elements or platforms of one or more types in whole or in part enable a shape programmable and or scaffolding system to which one or more elements of one or more types, including natural and or non-invention elements are affixed and or further form more one or more structures of one or more types

In one embodiment, one or more plasmonic elements and one or more types of associated electromagnetic radiation, including any energy elements and or platforms in one or more configurations form and or include optical elements such as, but not limited to, optics; optoelectronic elements; photoelectric elements; photodetectors; and photosensitive elements, which optical elements may also be coated or treated in whole or in part with materials that affect their optical properties.

In one embodiment, one or more plasmonic elements and or platforms in one or more configurations form and or include imaging elements and sensors, such as, but not limited to, CCDs and CMOS optical elements.

In one embodiment, one or more plasmonic elements and one or more types of associated electromagnetic radiation, including any energy elements and or platforms in one or more configurations include and or comprise photonic to electrical energy conversion elements.

In one embodiment, one or more plasmonic elements and or platforms form one or more electronic and or opto-electronic circuits, which circuits may also be composed of one or more other types of elements such as empty Fullerenes, endohedral Fullerenes, nanotubes, crystals, insulators, conductors, semiconductors, and or other materials, substances and devices; which circuits also may be coated in one or more surfactants and or cosurfactants and or other materials and substances.

In one embodiment, one or more plasmonic elements and one or more types of associated electromagnetic radiation, including any energy elements and or platforms are switched on or off and or change states by applying an electric field, and may also comprise one or more transistors or devices in another embodiment.

In another embodiment, one or more plasmonic elements and or platforms are mechanically assembled in one or more configurations via lithography, and or utilize other externally directed techniques and methods known the art, and or some combination thereof; and thereby form natural positions that are associated with electronic circuits and or information processing devices, such as atomic and molecular scale device design, their interconnection, nanofabrication and circuit architectures.

According to one illustrative embodiment, one or more plasmonic elements and one or more types of associated electromagnetic radiation, including any energy elements and or platforms comprise one or more crystal structures and elements, of one or more types.

According to one illustrative embodiment, one or more plasmonic elements and or platforms comprise one or more desiccated elements, of one or more types.

According to one illustrative embodiment, one or more elements and or platforms comprise one or more rehydration elements and or platforms, of one or more types.

According to one illustrative embodiment, one or more elements and or platforms are embedded and or incorporated into one or more materials, substances, devices, agents, devices, systems, organisms, and or mechanisms of one or more types.

In another illustrative embodiment, one or more plasmonic elements and one or more types of associated electromagnetic radiation, including any energy elements and or platforms comprise one or more magnetic nanoparticles of one or more types.

In other illustrative configurations, one or more plasmonic elements and or platforms are functionally linked via photonic, chemical, electromagnetic, electrical and/or quantum (non-classical) interactions of one or more types, including the Internet, to work and cooperate locally and/or remotely.

One or more invention elements and or platforms of one or more types may be encapsulated, packaged, stored, incorporated, and or utilize one or more methods known in the art, including for example, but not limited to: catheters; injections, including intramuscular injections; syringes; droppers and bulbs; pills; intravenous means; oral means; anal means; capsules; nanocapsules; nanoparticles; nano-devices; prescriptions; hospital and medical supplies; dental supplies; non-prescriptions; medications; over the counter products and remedies; alternative medicine supplies, systems, products and devices; hair care products; splints, casts, walkers, crutches, canes, wheelchairs, and other ambulatory aids; natural foods; vitamin and mineral supplements; first aid products; emergency health care procedures, systems, devices, and products, including combat medicine; health care products; grafts; skin patches; bandages; adhesives; wraps; masks; markers; powders; granules; geriatric care products; pediatric care products; diagnostic devices, systems, and products; medical imaging devices, systems, and products; telemedicine devices, systems, and products; in vivo monitoring systems, products, systems, and devices; in vitro monitoring systems, products, systems, and devices; laundry products; chemical, nuclear and biological sensors; sensors; bio-sensors; environmental sensors; combat systems, clothing, uniforms, and protective gear; food preparation products; food testing and safety devices, systems, and products; food storage wraps, systems, devices, and products; water treatment devices, systems and products; waste storage, management, and treatment systems and products; sewerage systems and products; plumbing systems and products; bed and bath products; animal care and veterinary products; animal feed; animal slaughter systems and products; cooking products; cookware; forensic devices, systems and products; home and office cleaning products; home products; office products; personal products; industrial products; home and office care products; paper products; personal hygiene products; sexual hygiene and safety products; sexual reproduction devices, systems, and products; sexual arousal products and devices; dental and dental care products; oral hygiene products, devices, and systems; robotic products, systems and devices; cybernetic devices; jewelry; novelties; solvents; agro-products; plants; animals; vehicles; biologicals; chemicals; cells; tissue; organs; proteins; liposomes; phages; micelles; peptides; antibodies; monoclonal antibodies; DNA; RNA; IRNA; siRNA; RISC; cloning; human contact; micro-electromechanical systems (MEMS) and other types of nano-systems; food utensils; tools; appliances; consumer electronics; paints and finishes; heating, ventilation and air conditioning systems; construction, building, home and office materials; water; milk; food and other edible or chewable substances and items; prostheses; food and drink additives and supplements; drinks; beverages; soaps; creams; ointments; salves; topical agents; cosmetics; beautifying agents; liquids; fluids; oils; gels; adhesives; aerosols; vapors; airborne methods; pumps; fragrances and perfumes; textiles; sporting and athletic goods and devices; physical work out and training systems, devices, and products; sports medicine systems, devices, and products; recreational products and gear; shoes, clothing, and apparel; eyewear; sprays; dyes; biological elements; organ transplants; implants; stents; prosthetic devices; artificial skin, blood, limbs, joints, bones, cells, eyes, organs, and other artificial body parts and biological elements; subcutaneous means; incisions; surgical means; and in-patient and out-patient medical procedures.

The above-described embodiments have been set forth to describe more completely and concretely the present invention, and are not to be construed as limiting the invention. It is further intended that all matter and the description and drawings be interpreted as illustrative and not in a limiting sense. That is, while various embodiments of the invention have been described in detail, other alterations, which will be apparent to those skilled in the prior art, are intended to be embraced within the spirit and scope of the invention.

In view of the foregoing, what is claimed is:

1. A plasmon energy composition to treat a disease, condition, or disorder with a plasmonic energy comprising: at least one of a portion of ultrabright fluorescent molecule elements that emit a collection of photons of a luminance equivalent to at least a luminance of photons emitted by at least one of a semiconductor quantum dot, and at least one of a metal element capable of photo-accepting at least some of the emitted photons, and at least one of a dielectric element disposed adjacent to a surface of the preceding metal, and administering to a subject in need thereof the preceding composition, wherein the composition generates at least one of a plasmonic energy, and the composition treats at least one of cell of the subject with the plasmonic energy.

2. (canceled)

3. (canceled)

4. (canceled)

5. The plasmon energy composition of claim 1, wherein the plasmonic energy is at least one of electrical, electromagnetic, photonic, plasmonic, thermal.

6. (canceled)

7. The plasmon energy composition of claim 1, further comprising a therapeutic agent.

8. The plasmon energy composition of claim 7, wherein the therapeutic agent is a drug for treating at least one of a disease, condition or disorder.

9. (canceled)

10. (canceled)

11. (canceled)

12. (canceled)

13. (canceled)

14. (canceled)

15. (canceled)

16. The plasmon energy composition of claim 1, wherein the composition is formulated for any suitable route and means of administration.

17. The plasmon energy composition of claim 1, wherein the composition is formulated as an adhesive, aerosols, a biologic, a capsule, a chemical compound, a coated, crystals, eye drops, gels, an injectable, liquids, oils, ointments, a peptide, a polymer, powders, a prosthetic, a protein, a non-cage coatomer, salves, soft galantine capsules, a stent, a controlled release, subcutaneous, surgical, syrups, a tablet, a topical, a vapor, or a water soluble composition.

18. The plasmon energy composition of claim 1, wherein the metal is comprised of at least some portion of alkali, cesium, cobalt, copper, francium, gold, iridium, iron, lithium, mercury, nickel, niobium, osmium, palladium, platinum, potassium, rhenium, rhodium, rubidium, ruthenium, silver, sodium, tantalum, titanium or zinc metal molecules or an alloy comprising two or more of the preceding metals.

19. The plasmon energy composition of claim 1, wherein the metal is configured as at least one of an symmetrical shell, asymmetrical shell, metal coated liposome, planar metal surface, metal surfaced element, metal composite, metal layer, metal nanoshell-hydrogel composite, multi-layer nanoshell, nano-star, nano-triangle, nanocube, nanoparticle, nanorod, nanoshell, nanosphere, noncontiguous shell or solid.

20. The plasmon energy composition of claim 1, wherein the dielectric element is comprised of at least one of an additive scattering particle, air, dopant, dye, gas, glass, metamaterial, peptide, polymer or protein.

21. A method for a plasmon energy composition to treat a disease, condition, or disorder with a plasmonic energy comprising: forming at least one of a portion of ultrabright fluorescent molecule elements that emit a collection of photons of a luminance equivalent to at least a luminance of photons emitted by at least one of a semiconductor quantum dot, and at least one of a metal element capable of photo-accepting at least some of the emitted photons, and at least one of a dielectric element disposed adjacent to a surface of the preceding metal, and administering to a subject in need thereof the preceding composition, wherein the composition generates at least one of a plasmonic energy, and the composition treats at least one of cell of the subject with the plasmonic energy.

22. The composition of claim 17, wherein one or more of the composition elements is bound together with at least some of the formulation elements.

23. The composition of claim 17, wherein one or more of the composition elements is incorporated into at least some of the formulation elements.

24. The composition of claim 17, wherein at least some portion of the formulation elements comprise a scaffold for supporting one or more of the elements.

25. The composition of claim 1, wherein the composition is stable with respect to dissociation.

26. A method for producing one or more of a plasmonic energy to treat at least one of a disease, condition or disorder of a subject, comprising administering the composition to the subject according to claim 1.

27. The method of claim 26, comprising producing at least one of electrical, electromagnetic, photonic, plasmonic, thermal energy.

28. The method of claim 26, comprising producing at least one of a wavelength of light energy.

29. The method of claim 28, comprising producing at least one of an ultraviolet, blue, red, infrared, near infrared, visible wavelength or combination thereof.

30. The method of claim 26, comprising producing at least one of a lasing energy.

31. The method of claim 26, comprising producing at least one of a quantum mechanical energy.

32. The method of claim 26, comprising propagating at least some of the energy by the composition and the composition acting as waveguide.

33. The method of claim 26, comprising storing at least some of the energy by the composition and the composition acting as capacitor.

34. The method of claim 26, comprising converting at least some of the energy by the composition and the composition acting as transducer.

35. A method for generating surface plasmons, comprising photo-accepting at least some photons by at least one of the metal according to claim 1.

36. The method of claim 35, comprising forming on at least one of a surface of the metal a collective excitation of conduction electrons.

37. The method of claim 36, comprising making available at least some of the conduction electrons as electromagnetic radiation or electrons.

38. The method of claim 37, comprising confining at least some of the electromagnetic radiation or electrons to the surface of the metal according to claim 36.

39. The method of claim 38, comprising using at least one of the dielectric according to claim 1 to confine at least some of the electromagnetic radiation or electrons to the surface of the metal.

40. The method of claim 39, comprising producing at least some of surface plasmon polaritons at the dielectric-metal interface for use in treating the subject according to claim 1.

41. A method for treating a disease, condition, or disorder, comprising administering the composition to the subject according to claim 1.

42. A method for treating at least one of cell disease, condition, or disorder, comprising formulating the composition according to claim 1 as a supplemental drug for use by other drugs.

43. A method for targeting at least one of cell disease, condition, or disorder, comprising administering the composition to the subject according to claim 1.

44. A method for controlling the composition according to claim 1, comprising using at least one of algorithms.
